SEQUENCE LISTING (87 sequences)

SEQ ID NO: 1 - protein, 359 aa, unidentified
  REGION 1..359: note = DNA ligase from Enterobacteria phage T7 (unmodified)
MMNIKTNPFKAVSFVESAIKKALDNAGYLIAEIKYDGVRGNICVDNTANSYWLSRVSKTIPALEHLNGFDVRWKRLLNDDRCFYKDGFMLDGELMVKGVDFNTGSGLLRTKWTDTKNQEFHEELFVEPIRKKDKVPFKLHTGHLHIKLYAILPLHIVESGEDCDVMTLLMQEHVKNMLPLLQEYFPEIEWQAAESYEVYDMVELQQLYEQKRAEGHEGLIVKDPMCIYKRGKKSGWWKMKPENEADGIIQGLVWGTKGLANEGKVIGFEVLLESGRLVNATNISRALMDEFTETVKEATLSQWGFFSPYGIGDNDACTINPYDGWACQISYMEETPDGSLRHPSFVMFRGTEDNPQEKM

SEQ ID NO: 2 - protein, 324 aa, unidentified
  REGION 1..324: note = DNA ligase from Vibrio phage VP3 (unmodified)
MTNFKINPFKAVSFKESAIQKVMELGGVIAAVKYDGLRCHLVITPTVDIQGKPAARMVAISRTDKAIPALRELFISDEHKILLGQLLEESLYPEGLVIDGEVMVKGVDFNTGSGMLRRKEAIHVSKLEYKVYGVLPLSAIKASPDTEIEVPYCVMYEQVKVLMHQLWELLPEMDWKQEETYEVYDMASLEELFKKVRAEGHEGLIIKDPMTNWKRGKKTGYWKMKPEGEIDGTVVGVNWGTVGLANEGKVIGFQVLLENGVVVDANGITQEQMEEYTNLVYKTGHDDCFNGRPVQVKYMEKTPKGSLRHPSFQRWRDLEGAGGS

SEQ ID NO: 3 - protein, 340 aa, unidentified
  REGION 1..340: note = DNA ligase from Stenotrophomonas phage IME15 (unmodified)
MMNIKTNPYKAVSFNEKAVEKALKRSHYLIADIKYDGVRGNTLVKPSQGACWLSRVSKPIPAIEHLNDGAARWDSLLKDNRCMFPTGFMLDGELMVDGVDFNTGSGLLRTKWLDSKNQEFHNEFLPRNVKKSEKIPFRLDAGLLKVVIYAVLPIEVAESGDDYPVKNLLMQEHVKNMLPLLRAHFPEISWQAAESHEVYDMTELSALYEMKREEGHEGLIVKDPSDIYKRGKKTGWWKMKPENEADGVIQGLVWGTKGLANEGKVIGFEVLLESGRVVNACNIAQSLMNEFTAAVETHGEDYYNGWTCQISYMEETPDGSLRHPSFVMFRGTEAEPTEKM

SEQ ID NO: 4 - protein, 308 aa, unidentified
  REGION 1..308: note = DNA ligase from Caulobacter phage Percy (unmodified)
MSKDYIAGKFVEVRKLAKTTGLGKTLHKVSLDEAFEALGKEYEAQSKHDGCSGVVFLHADDTKPTTVLSRTGEECLSMQMQGRLLRDDFDTVVSKYGPLVLLTEAWWPGKGEFAEISGAFRKKSEQRPRLMFVVSDILTLEEFNAGKTSVPYRARMDRGRGVQPKTAAWTWTTRHPPGTYGDPRDLCAKYVRGGGFDGVVLVDLGAGWVKEEDKSGRKIKVKNELSFALRVKEVKVATGEKTGRDVYTLVVGFDGRDLGVGSGVPHKVEDVPKVGDIVEIVAMDYSSDGLLREPRYKGIRADVIKEDR

SEQ ID NO: 5 - protein, 343 aa, unidentified
  REGION 1..343: note = DNA ligase from Morganella phage vB_MmoP_MP2 (unmodified)
MLNIKTNPYKAVSFNENAIMKAAEKAGSLIADIKYDGVRLNLVAKHTGGFFLSRTSKVIPALEHLCGYSERWENFLADERQIYKGGIMIDGEAMIKGVDFNTGSGILRTKWLSKKNVKFTTQPDYEIPRGMGKVPFELDPQNLKVVVYAVLPLDVVESGEDHDVMNCVMPYHVDSVIELLVEYFPEIDWVRAESHDVYSMMQLDKLYEFVRERGDEGLVVKDPLDIYKRGKKTGWWKMKPENEADGTIVGLVWGTKGKANEGKVIGFEVLLENGRVVNATNISQALMEEFTLNVKTQELNGDTEAYIGWACQITYMEETPDGSLRHPSFDKFRGTEDNPKEKM

SEQ ID NO: 6 - protein, 341 aa, unidentified
  REGION 1..341: note = DNA ligase from Klebsiella phage KP32 (unmodified)
MMNIKTNPFKAVSFVRSAIEKALETSGYLIADTKHDGVRGNICVDNTANAAWLSRVSKTVPALEHLNGFDMRWQKLLKDDRWIFPDGFMLDSELMVKGVDFNTGSGLLRTKWLKSKNYEFSTYAIEKEWDNPKGKIPFHLNSADLKVVLYDIIPLDIIESGDDYNVMTLLRLEHVKVALPVLQDHFPEVEWCLSESHEVYDMDELDALYRQKREEGHEGLVVKDPRGIYKRGKKSGWFKCKPNDEADGIIQGVNWGTPGLANEGKVIGFSVLLETGRLVDANNISRALMDEFTANVKEHGEEYYNGWSCQIGYMEETKDGSLRHPSFTCFRGTEDNPSEKI

SEQ ID NO: 7 - protein, 336 aa, unidentified
  REGION 1..336: note = DNA ligase from Vibrio phage N4 (unmodified)
MTNFKINPFKAVSFKESAIQKVMELGGVIAAVKYDGLRCHLVITPTADIQGKPAARMVAISRTDKAIPALRELFISAEHKILLGQLLEESLYPEGLVIDGEVMVKGVDFNTGSGMLRRKEAIHVSKLAYKVYGVLPLNALKASPDAEIEVPYCVMYEQVKVLMHQLWELLPEMDWKQEETYEVYDMASLEELFKKVRAEGHEGLIIKDPMTNWKRGKKTGYWKMKPEGEIDGTVVGVNWGTVGLANEGKVIGFQVLLENGVVVDANGITQEQMEEYTNSIKGYGQYHYCDQYTAEEAVFHGRPVQVKYMEKTPKGSLRHPSFQRWRDLEGAEGVKA

SEQ ID NO: 8 - protein, 338 aa, unidentified
  REGION 1..338: note = DNA ligase from Salmonella phage phiSG-JL2 (unmodified)
MNIFKTNPFKAVSFVESAVKKALETSGYLIADIKYDGVRGNIVVDNVAEAAWLSRVSKFIPALEHLNGFDKRWQQLMNDDRCIFPDGFMLDGELMVKGVDFNTGSGLLRTKWLKKNNFMFDRGGVEPLKGSKVAFELDPKLLSVRLYAVLPIHIAESGEDYDVQNLLMPYHVEAMRSLLVEYFPEIEWLIAETYEVYDMDSLTELYEEKRAEGHEGLIVKDPQGIYKRGKKSGWWKLKPECEADGIIQGVNWGTEGLANEGKVIGFSVLLETGRLVDANNISRALMDEFTANVKAHGEDFYNGWACQVNYMEETPDGSLRHPSFEKFRGTEDNPQEKM

SEQ ID NO: 9 - protein, 356 aa, unidentified
  REGION 1..356: note = DNA ligase from Escherichia phage 64795_ec1 (unmodified)
MNIKTSPFKAVSFVESAVKKALDNAGYLIVDCKYDGVRGNICVDNTANSCWLSRVSKTIPALEHLNGFDVRWKSLLNDDRCFYKDGFMLDGELMVKGVDFNTGSGLLRTKWLNKKNFEFDVGGVPPLKGSKVEFALDTKRLSVRLYAILPLHIVESGEDCDVMTLLMQEHVKNMLPLLQEYFPEIEWQAAESYEVYDMVELQQLYEKKRAEGHEGLIVKDPMCIYKRGKKSGWWKMKPENEADGIIQGLVWGTEGLANEGKVIGFEVLLESGRVVNATNISRALMDEITETVKEATLSQWGFFSPYGIGDNDACTINPYDGWSCQISYMEETPDGSLRHPSFVMFRGTEDNPQEKM

SEQ ID NO: 10 - protein, 232 aa, unidentified
  REGION 1..232: note = Single-stranded DNA binding protein from Enterobacteria phage T7 (unmodified)
MAKKIFTSALGTAEPYAYIAKPDYGNEERGFGNPRGVYKVDLTIPNKDPRCQRMVDEIVKCHEEAYAAAVEEYEANPPAVARGKKPLKPYEGDMPFFDNGDGTTTFKFKCYASFQDKKTKETKHINLVVVDSKGKKMEDVPIIGGGSKLKVKYSLVPYKWNTAVGASVKLQLESVMLVELATFGGGEDDWADEVEENGYVASGSAKASKPRDEESWDEDDEESEEADEDGDF

SEQ ID NO: 11 - protein, 217 aa, unidentified
  REGION 1..217: note = Single-stranded DNA binding protein from Delftia phage IME-DE1 (unmodified)
MADKPTVIVTPQATVFGWVNILKPDTKYNAAGDYKIKVRIPKDTKGLAAQLEKIEEAREKAKAAWLKEPKNKGKRLKEADLPFYEDDEGNIVMSFKSVASWTDAKSGETRYRQIPVFGGAGRIDPKQVPQFGEGSIVRVAYTISAFANAAVGAGASLRIDSVKLIEVKQFSGGGAANHFGDDEEGYVPESGGDDSDPYGNDDTGSDAGGSAGDNDEF

SEQ ID NO: 12 - protein, 232 aa, unidentified
  REGION 1..232: note = Single-stranded DNA binding protein from Kluyvera phage Kvp1 (unmodified)
MAFAKRKVFTSARGVAEPYCYLAKPDFGSGDFKNERGVYKVSLTVSNDDPRCQKMIDEIVEAHETDYAARLEEYEANPPKVVKGKKPLKPYVGDMPFMDNEDGTTTFNFKCYGSFTDKKTGENKPIDLAIVDSKGKRIRGERPAISGGSELKIKYTLFPYGWSAVAGASVKLQLDSVMLIKLVEFGGGDDDWSDETEEDGYEADEAQTRKPQNGSGWNEEPEEEEDDEGGDF

SEQ ID NO: 13 - protein, 230 aa, unidentified
  REGION 1..230: note = Single-stranded DNA binding protein from Stenotrophomonas phage IME15 (unmodified)
MAKKIFTSALGTAEPYAYLAKPDYGNEERGFGNPRGVYKVDLTIPNKDPRCQRMVDEIVKCHEEAYAAAVEEYEANPPAVARGKKPLKPYEGDLPFFDNGDGTTTFKFKCYASFQDKKTKETKHINLVVVDSKGKKLEEVPIIGGGSKLKVKYSLVPYKWNTAVGASVKLQLESVMLVELATFGGGEDDWADEVEEGGYVASGFARSKPRDEEDWSADTEEESAGDDEDF

SEQ ID NO: 14 - protein, 193 aa, unidentified
  REGION 1..193: note = Single-stranded DNA binding protein from Synechococcus phage P60 (unmodified)
MADRNRYVFEATLEGFINVYEDEGKFNNRCFSFKLPKDVIEAAEADREELLEWAKSKAPNPRRCEVALSKWDDEGLVKYSYSEEKPKAIPVFVDTEGSLIDKAVLKDVRKGTKVKLIVQQGPYVFGNKVGTRLLVKGVQIIELASGNGSVDSGDMSAEDVAAIFGTTSGFKQSDPQVRKAAEAEPETTESYDF

SEQ ID NO: 15 - protein, 187 aa, unidentified
  REGION 1..187: note = Single-stranded DNA binding protein from uncultured phage MedDCM-OCT-S04-C24 (unmodified)
MPRTIHKTEQPVILEGYQAVLKPSKFGYSLSAIVDGSMVDALETDREESLQWAQGKLKNPKRSVLKPEPWEEVADNQFKVKFSWNEENRPPVVDTEGTPVTDENTPMYSGSRVNLAFYQKPYILKDGVTYGTSLKLVGVQLVSLNSGAGVDTGDMAAEDVAALFGKTEGFKANDPAVTVLPGSDDDF

SEQ ID NO: 16 - protein, 239 aa, unidentified
  REGION 1..239: note = Single-stranded DNA binding protein from Morganella phage vB_MmoP_MP2 (unmodified)
MSGIDYRKKVFTTPVGTCEPYAYIRQPDFGGEGFKNPRGTYKVDLTLSKDDAGCQKMMDAIVACHEESYAARVADYEENPPKVQRGKKPLVPYEGDMPFIDNDDGTVTFKFKAYGSFTDKKTGENREIKLLVVDSKGKKLEPVPIIAQGSQLKVKFKMVPYGWTPTVGASVKLQLESIMVVELAEFGGGSDLWGDDDFEDGFSADQNPAAKSRKGDDNEDDEPWDTDGDDEPTDDADDF

SEQ ID NO: 17 - protein, 231 aa, unidentified
  REGION 1..231: note = Single-stranded DNA binding protein from Klebsiella phage K11 (unmodified)
MAFAKKKIYTTKIGTCEPYAYFNKPDYGGEGFENPRGTYKGYVTFKNEDCQELVDLIVKTHEENYAARLEAHEANPPKVQKGKKPLKPYEGDMPFFDNGDGTTTFNFKCYGSYEDKKTGETKKIVLGVVDAKGKRIQDVPIIGGGSKVKIRFSLVPYGWSAVAGASVKLQLEGVMLVELATFGGGEDDWADEAVEGGYEADESRSRKPQEDPEDWSGEEADEGEAEEDDDF

SEQ ID NO: 18 - protein, 231 aa, unidentified
  REGION 1..231: note = Single-stranded DNA binding protein from Klebsiella phage KP32 (unmodified)
MAFAKKKIYTTKIGTCEPYAYFNKPDYGGEGFENPRGTYKGSVTFKNEDCQELVDLIVKTHEENYAARLEAHEANPPKVQKGKKPLKPYEGDMPFFDNGDGTTTFNFKCYGSYEDKKTGETKKIVLGVVDAKGKRIQDVPIIGGGSKVKIRFSLVPYGWSAVAGASVKLQLEGVMLVELATFGGGEDDWADEAVEGGYEADEPRSRKPQEDPEDWSGEEADEGEAEEDDDF

SEQ ID NO: 19 - protein, 231 aa, unidentified
  REGION 1..231: note = Single-stranded DNA binding protein from Escherichia phage vB_EcoP_GA2A (unmodified)
MAKEQLKTFTTPVAGIVEPYAWLNKADTKFNERGEHKVNLTFDLSDPKVRMMIDVLQKIHDDAYAKALADHEKNPPQVQRGKKPIEPREGDMPWIENGDGTVTMKFKCFASYLKDGKSEPIVLRFYDTDAKLIRDVPNIGAGSKLKVKFKVLPFKWNAATGASVKLQLESCLLVELKEWKGDGAGGDGGWGDDEDLGTGYKASTDGDFGSDDFGEDADGGDDSASGGDYDF

SEQ ID NO: 20 - protein, 231 aa, unidentified
  REGION 1..231: note = Single-stranded DNA binding protein from Yersinia phage vB_YenP_AP10 (unmodified)
MAIKRKVFTTPKGTSEPYAYIAKPDFGGQGFENPRGTYKVSLTLPNDSKTQAMIDAIVACHEEDYARRLEEHEANPPKVVKGKKPLQPYTGDLPFFDNGDGTTTFNFKAHASYEDKKTGESKKINLSVVDSQGKRINDVPLIAGGSELKVKFSMVPYGWTNVAGASVKLQLEGVMIISLATFGGESDDWGDEIEEGGYTADNSRSRNPEQEEGHDFGSDDESGDDDESGDF

SEQ ID NO: 21 - protein, 232 aa, unidentified
  REGION 1..232: note = Single-stranded DNA binding protein from Salmonella phage phiSG-JL2 (unmodified)
MAGFKKKIYTSGLGTAEPYAYLSKPDYGNEERGFGNPRGVYKVDLTLSNKDPRCQAMVDEIVKTHEEAYAAAVEEFEANPPQVQRGKKPLKPYEGDMPFFDNGDGTTTFKFKCYASFQDKKTKETKHINLVVVDSKGKKIQEVPIIGGGSKLKVKYSLVPYKWNTAVGASVKLQLESVMLVELATFGGGGEDEWADEVEDGGYTASESRQSRDEQEWQEDEHEETPDDNEDF

SEQ ID NO: 22 - protein, 234 aa, unidentified
  REGION 1..234: note = Single-stranded DNA binding protein from Salmonella phage Vi06 (unmodified)
MAKKIYTSALGTAEPYAYITKPDYGNEERGFGNPRGVYKVDLTIPNKDPRCQRMVDEIVKCHEEAYAAAVEEYEANPPAVARGKKPLRPYEGDMPFFDNGDGTTTFKFKAYASYQDKKTKETKHINMVVVDSKGKKIEDVPIIGGGSKLKVKYSLIPYKWNTAVGASVKLQLESVMLVELATFGGGEDDWADEVEENGYVASGSAKASKPRDEESWSQDSDEDAYGEGDSDGDF

SEQ ID NO: 23 - protein, 232 aa, unidentified
  REGION 1..232: note = Single-stranded DNA binding protein from Enterobacteria phage 13a (unmodified)
MAKKIFTSALGTAEPYAYIAKPDYGNEERGFGNPRGVYKVDLTIPNKDPRCQRMVDEIVKCHEEAYAAAVEEYEANPPAVARGKKPLKPYEGDMPFFDNGDGTTTFKFKCYASFQDKKTKETKHINLVVVDSKGKKMEDVPIIGGGSKLKVKYSLVPYKWNTAVGASVKLQLESVMLVELATFGGGEDDWADEVEENGYVASGSAKASKPRDEESWDEDDEGSYEEDSDGDF

SEQ ID NO: 24 - protein, 232 aa, unidentified
  REGION 1..232: note = Single-stranded DNA binding protein from Escherichia phage 64795_ec1 (unmodified)
MAKKIFTSALGTAEPYAYIGKPDYGNEERGFGNPRGVYKVDLTIPNKDPRCQRMVDEIVKCHEEAYAAAVEEYEANPPAVARGKKPLKPYEGDMPFFDNGDGTTTFKFKCYASFQDKKTKETKHINLVVVDSKGKKMEDVPIIGGGSKLKVKYSLVPYKWNTAVGASVKLQLESVMLVELATFGGGEDDWADEVEENGYVASGSAKASKPRDEESWDEDDEESDEVDEDGDF

SEQ ID NO: 25 - protein, 346 aa, unidentified
  REGION 1..346: note = DNA ligase from Enterobacteria phage T3 (unmodified)
MNIFNTNPFKAVSFVESAVKKALETSGYLIADCKYDGVRGNIVVDNVAEAAWLSRVSKFIPALEHLNGFDKRWQQLLNDDRCIFPDGFMLDGELMVKGVDFNTGSGLLRTKWVKRDNMGFHLTNVPTKLTPKGREVIDGKFEFHLDPKRLSVRLYAVMPIHIAESGEDYDVQNLLMPYHVEAMRSLLVEYFPEIEWLIAETYEVYDMDSLTELYEEKRAEGHEGLIVKDPQGIYKRGKKSGWWKLKPECEADGIIQGVNWGTEGLANEGKVIGFSVLLETGRLVDANNISRALMDEFTSNVKAHGEDFYNGWACQVNYMEATPDGSLRHPSFEKFRGTEDNPQEKM

SEQ ID NO: 26 - protein, 487 aa, unidentified
  REGION 1..487: note = DNA ligase from Enterobacteria phage T4 (unmodified)
MILKILNEIASIGSTKQKQAILEKNKDNELLKRVYRLTYSRGLQYYIKKWPKPGIATQSFGMLTLTDMLDFIEFTLATRKLTGNAAIEELTGYITDGKKDDVEVLRRVMMRDLECGASVSIANKVWPGLIPEQPQMLASSYDEKGINKNIKFPAFAQLKADGARCFAEVRGDELDDVRLLSRAGNEYLGLDLLKEELIKMTAEARQIHPEGVLIDGELVYHEQVKKEPEGLDFLFDAYPENSKAKEFAEVAESRTASNGIANKSLKGTISEKEAQCMKFQVWDYVPLVEIYSLPAFRLKYDVRFSKLEQMTSGYDKVILIENQVVNNLDEAKVIYKKYIDQGLEGIILKNIDGLWENARSKNLYKFKEVIDVDLKIVGIYPHRKDPTKAGGFILESECGKIKVNAGSGLKDKAGVKSHELDRTRIMENQNYYIGKILECECNGWLKSDGRTDYVKLFLPIAIRLREDKTKANTFEDVFGDFHEVTGL

SEQ ID NO: 27 - protein, 367 aa, synthetic construct
  REGION 1..367: note = Synthetic: DNA ligase from Enterobacteria phage T7 (with N-terminal His6 tag)
MHHHHHHGSMNIKTNPFKAVSFVESAIKKALDNAGYLIAEIKYDGVRGNICVDNTANSYWLSRVSKTIPALEHLNGFDVRWKRLLNDDRCFYKDGFMLDGELMVKGVDFNTGSGLLRTKWTDTKNQEFHEELFVEPIRKKDKVPFKLHTGHLHIKLYAILPLHIVESGEDCDVMTLLMQEHVKNMLPLLQEYFPEIEWQAAESYEVYDMVELQQLYEQKRAEGHEGLIVKDPMCIYKRGKKSGWWKMKPENEADGIIQGLVWGTKGLANEGKVIGFEVLLESGRLVNATNISRALMDEFTETVKEATLSQWGFFSPYGIGDNDACTINPYDGWACQISYMEETPDGSLRHPSFVMFRGTEDNPQEKM

SEQ ID NO: 28 - protein, 332 aa, synthetic construct
  REGION 1..332: note = Synthetic: DNA ligase from Vibrio phage VP3 (with N-terminal His6 tag)
MHHHHHHGSTNFKINPFKAVSFKESAIQKVMELGGVIAAVKYDGLRCHLVITPTVDIQGKPAARMVAISRTDKAIPALRELFISDEHKILLGQLLEESLYPEGLVIDGEVMVKGVDFNTGSGMLRRKEAIHVSKLEYKVYGVLPLSAIKASPDTEIEVPYCVMYEQVKVLMHQLWELLPEMDWKQEETYEVYDMASLEELFKKVRAEGHEGLIIKDPMTNWKRGKKTGYWKMKPEGEIDGTVVGVNWGTVGLANEGKVIGFQVLLENGVVVDANGITQEQMEEYTNLVYKTGHDDCFNGRPVQVKYMEKTPKGSLRHPSFQRWRDLEGAGGS

SEQ ID NO: 29 - protein, 348 aa, synthetic construct
  REGION 1..348: note = Synthetic: DNA ligase from Stenotrophomonas phage IME15 (with N-terminal His6 tag)
MHHHHHHGSMNIKTNPYKAVSFNEKAVEKALKRSHYLIADIKYDGVRGNTLVKPSQGACWLSRVSKPIPAIEHLNDGAARWDSLLKDNRCMFPTGFMLDGELMVDGVDFNTGSGLLRTKWLDSKNQEFHNEFLPRNVKKSEKIPFRLDAGLLKVVIYAVLPIEVAESGDDYPVKNLLMQEHVKNMLPLLRAHFPEISWQAAESHEVYDMTELSALYEMKREEGHEGLIVKDPSDIYKRGKKTGWWKMKPENEADGVIQGLVWGTKGLANEGKVIGFEVLLESGRVVNACNIAQSLMNEFTAAVETHGEDYYNGWTCQISYMEETPDGSLRHPSFVMFRGTEAEPTEKM

SEQ ID NO: 30 - protein, 316 aa, synthetic construct
  REGION 1..316: note = Synthetic: DNA ligase from Caulobacter phage Percy (with N-terminal His6 tag)
MHHHHHHGSSKDYIAGKFVEVRKLAKTTGLGKTLHKVSLDEAFEALGKEYEAQSKHDGCSGVVFLHADDTKPTTVLSRTGEECLSMQMQGRLLRDDFDTVVSKYGPLVLLTEAWWPGKGEFAEISGAFRKKSEQRPRLMFVVSDILTLEEFNAGKTSVPYRARMDRGRGVQPKTAAWTWTTRHPPGTYGDPRDLCAKYVRGGGFDGVVLVDLGAGWVKEEDKSGRKIKVKNELSFALRVKEVKVATGEKTGRDVYTLVVGFDGRDLGVGSGVPHKVEDVPKVGDIVEIVAMDYSSDGLLREPRYKGIRADVIKEDR

SEQ ID NO: 31 - protein, 351 aa, synthetic construct
  REGION 1..351: note = Synthetic: DNA ligase from Morganella phage vB_MmoP_MP2 (with N-terminal His6 tag)
MHHHHHHGSLNIKTNPYKAVSFNENAIMKAAEKAGSLIADIKYDGVRLNLVAKHTGGFFLSRTSKVIPALEHLCGYSERWENFLADERQIYKGGIMIDGEAMIKGVDFNTGSGILRTKWLSKKNVKFTTQPDYEIPRGMGKVPFELDPQNLKVVVYAVLPLDVVESGEDHDVMNCVMPYHVDSVIELLVEYFPEIDWVRAESHDVYSMMQLDKLYEFVRERGDEGLVVKDPLDIYKRGKKTGWWKMKPENEADGTIVGLVWGTKGKANEGKVIGFEVLLENGRVVNATNISQALMEEFTLNVKTQELNGDTEAYIGWACQITYMEETPDGSLRHPSFDKFRGTEDNPKEKM

SEQ ID NO: 32 - protein, 349 aa, synthetic construct
  REGION 1..349: note = Synthetic: DNA ligase from Klebsiella phage KP32 (with N-terminal His6 tag)
MHHHHHHGSMNIKTNPFKAVSFVRSAIEKALETSGYLIADTKHDGVRGNICVDNTANAAWLSRVSKTVPALEHLNGFDMRWQKLLKDDRWIFPDGFMLDSELMVKGVDFNTGSGLLRTKWLKSKNYEFSTYAIEKEWDNPKGKIPFHLNSADLKVVLYDIIPLDIIESGDDYNVMTLLRLEHVKVALPVLQDHFPEVEWCLSESHEVYDMDELDALYRQKREEGHEGLVVKDPRGIYKRGKKSGWFKCKPNDEADGIIQGVNWGTPGLANEGKVIGFSVLLETGRLVDANNISRALMDEFTANVKEHGEEYYNGWSCQIGYMEETKDGSLRHPSFTCFRGTEDNPSEKI

SEQ ID NO: 33 - protein, 344 aa, synthetic construct
  REGION 1..344: note = Synthetic: DNA ligase from Vibrio phage N4 (with N-terminal His6 tag)
MHHHHHHGSTNFKINPFKAVSFKESAIQKVMELGGVIAAVKYDGLRCHLVITPTADIQGKPAARMVAISRTDKAIPALRELFISAEHKILLGQLLEESLYPEGLVIDGEVMVKGVDFNTGSGMLRRKEAIHVSKLAYKVYGVLPLNALKASPDAEIEVPYCVMYEQVKVLMHQLWELLPEMDWKQEETYEVYDMASLEELFKKVRAEGHEGLIIKDPMTNWKRGKKTGYWKMKPEGEIDGTVVGVNWGTVGLANEGKVIGFQVLLENGVVVDANGITQEQMEEYTNSIKGYGQYHYCDQYTAEEAVFHGRPVQVKYMEKTPKGSLRHPSFQRWRDLEGAEGVKA

SEQ ID NO: 34 - protein, 346 aa, synthetic construct
  REGION 1..346: note = Synthetic: DNA ligase from Salmonella phage phiSG-JL2 (with N-terminal His6 tag)
MHHHHHHGSNIFKTNPFKAVSFVESAVKKALETSGYLIADIKYDGVRGNIVVDNVAEAAWLSRVSKFIPALEHLNGFDKRWQQLMNDDRCIFPDGFMLDGELMVKGVDFNTGSGLLRTKWLKKNNFMFDRGGVEPLKGSKVAFELDPKLLSVRLYAVLPIHIAESGEDYDVQNLLMPYHVEAMRSLLVEYFPEIEWLIAETYEVYDMDSLTELYEEKRAEGHEGLIVKDPQGIYKRGKKSGWWKLKPECEADGIIQGVNWGTEGLANEGKVIGFSVLLETGRLVDANNISRALMDEFTANVKAHGEDFYNGWACQVNYMEETPDGSLRHPSFEKFRGTEDNPQEKM

SEQ ID NO: 35 - protein, 364 aa, synthetic construct
  REGION 1..364: note = Synthetic: DNA ligase from Escherichia phage 64795_ec1 (with N-terminal His6 tag)
MHHHHHHGSNIKTSPFKAVSFVESAVKKALDNAGYLIVDCKYDGVRGNICVDNTANSCWLSRVSKTIPALEHLNGFDVRWKSLLNDDRCFYKDGFMLDGELMVKGVDFNTGSGLLRTKWLNKKNFEFDVGGVPPLKGSKVEFALDTKRLSVRLYAILPLHIVESGEDCDVMTLLMQEHVKNMLPLLQEYFPEIEWQAAESYEVYDMVELQQLYEKKRAEGHEGLIVKDPMCIYKRGKKSGWWKMKPENEADGIIQGLVWGTEGLANEGKVIGFEVLLESGRVVNATNISRALMDEITETVKEATLSQWGFFSPYGIGDNDACTINPYDGWSCQISYMEETPDGSLRHPSFVMFRGTEDNPQEKM

SEQ ID NO: 36 - protein, 240 aa, synthetic construct
  REGION 1..240: note = Synthetic: Single-stranded DNA binding protein from Enterobacteria phage T7 (with N-terminal His6 tag)
MHHHHHHGSAKKIFTSALGTAEPYAYIAKPDYGNEERGFGNPRGVYKVDLTIPNKDPRCQRMVDEIVKCHEEAYAAAVEEYEANPPAVARGKKPLKPYEGDMPFFDNGDGTTTFKFKCYASFQDKKTKETKHINLVVVDSKGKKMEDVPIIGGGSKLKVKYSLVPYKWNTAVGASVKLQLESVMLVELATFGGGEDDWADEVEENGYVASGSAKASKPRDEESWDEDDEESEEADEDGDF

SEQ ID NO: 37 - protein, 225 aa, synthetic construct
  REGION 1..225: note = Synthetic: Single-stranded DNA binding protein from Delftia phage IME-DE1 (with N-terminal His6 tag)
MHHHHHHGSADKPTVIVTPQATVFGWVNILKPDTKYNAAGDYKIKVRIPKDTKGLAAQLEKIEEAREKAKAAWLKEPKNKGKRLKEADLPFYEDDEGNIVMSFKSVASWTDAKSGETRYRQIPVFGGAGRIDPKQVPQFGEGSIVRVAYTISAFANAAVGAGASLRIDSVKLIEVKQFSGGGAANHFGDDEEGYVPESGGDDSDPYGNDDTGSDAGGSAGDNDEF

SEQ ID NO: 38 - protein, 240 aa, synthetic construct
  REGION 1..240: note = Synthetic: Single-stranded DNA binding protein from Kluyvera phage Kvp1 (with N-terminal His6 tag)
MHHHHHHGSAFAKRKVFTSARGVAEPYCYLAKPDFGSGDFKNERGVYKVSLTVSNDDPRCQKMIDEIVEAHETDYAARLEEYEANPPKVVKGKKPLKPYVGDMPFMDNEDGTTTFNFKCYGSFTDKKTGENKPIDLAIVDSKGKRIRGERPAISGGSELKIKYTLFPYGWSAVAGASVKLQLDSVMLIKLVEFGGGDDDWSDETEEDGYEADEAQTRKPQNGSGWNEEPEEEEDDEGGDF

SEQ ID NO: 39 - protein, 238 aa, synthetic construct
  REGION 1..238: note = Synthetic: Single-stranded DNA binding protein from Stenotrophomonas phage IME15 (with N-terminal His6 tag)
MHHHHHHGSAKKIFTSALGTAEPYAYLAKPDYGNEERGFGNPRGVYKVDLTIPNKDPRCQRMVDEIVKCHEEAYAAAVEEYEANPPAVARGKKPLKPYEGDLPFFDNGDGTTTFKFKCYASFQDKKTKETKHINLVVVDSKGKKLEEVPIIGGGSKLKVKYSLVPYKWNTAVGASVKLQLESVMLVELATFGGGEDDWADEVEEGGYVASGFARSKPRDEEDWSADTEEESAGDDEDF

SEQ ID NO: 40 - protein, 201 aa, synthetic construct
  REGION 1..201: note = Synthetic: Single-stranded DNA binding protein from Synechococcus phage P60 (with N-terminal His6 tag)
MHHHHHHGSADRNRYVFEATLEGFINVYEDEGKFNNRCFSFKLPKDVIEAAEADREELLEWAKSKAPNPRRCEVALSKWDDEGLVKYSYSEEKPKAIPVFVDTEGSLIDKAVLKDVRKGTKVKLIVQQGPYVFGNKVGTRLLVKGVQIIELASGNGSVDSGDMSAEDVAAIFGTTSGFKQSDPQVRKAAEAEPETTESYDF

SEQ ID NO: 41 - protein, 195 aa, synthetic construct
  REGION 1..195: note = Synthetic: Single-stranded DNA binding protein from uncultured phage MedDCM-OCT-S04-C24 (with N-terminal His6 tag)
MHHHHHHGSPRTIHKTEQPVILEGYQAVLKPSKFGYSLSAIVDGSMVDALETDREESLQWAQGKLKNPKRSVLKPEPWEEVADNQFKVKFSWNEENRPPVVDTEGTPVTDENTPMYSGSRVNLAFYQKPYILKDGVTYGTSLKLVGVQLVSLNSGAGVDTGDMAAEDVAALFGKTEGFKANDPAVTVLPGSDDDF

SEQ ID NO: 42 - protein, 247 aa, synthetic construct
  REGION 1..247: note = Synthetic: Single-stranded DNA binding protein from Morganella phage vB_MmoP_MP2 (with N-terminal His6 tag)
MHHHHHHGSSGIDYRKKVFTTPVGTCEPYAYIRQPDFGGEGFKNPRGTYKVDLTLSKDDAGCQKMMDAIVACHEESYAARVADYEENPPKVQRGKKPLVPYEGDMPFIDNDDGTVTFKFKAYGSFTDKKTGENREIKLLVVDSKGKKLEPVPIIAQGSQLKVKFKMVPYGWTPTVGASVKLQLESIMVVELAEFGGGSDLWGDDDFEDGFSADQNPAAKSRKGDDNEDDEPWDTDGDDEPTDDADDF

SEQ ID NO: 43 - protein, 239 aa, synthetic construct
  REGION 1..239: note = Synthetic: Single-stranded DNA binding protein from Klebsiella phage K11 (with N-terminal His6 tag)
MHHHHHHGSAFAKKKIYTTKIGTCEPYAYFNKPDYGGEGFENPRGTYKGYVTFKNEDCQELVDLIVKTHEENYAARLEAHEANPPKVQKGKKPLKPYEGDMPFFDNGDGTTTFNFKCYGSYEDKKTGETKKIVLGVVDAKGKRIQDVPIIGGGSKVKIRFSLVPYGWSAVAGASVKLQLEGVMLVELATFGGGEDDWADEAVEGGYEADESRSRKPQEDPEDWSGEEADEGEAEEDDDF

SEQ ID NO: 44 - protein, 239 aa, synthetic construct
  REGION 1..239: note = Synthetic: Single-stranded DNA binding protein from Klebsiella phage KP32 (with N-terminal His6 tag)
MHHHHHHGSAFAKKKIYTTKIGTCEPYAYFNKPDYGGEGFENPRGTYKGSVTFKNEDCQELVDLIVKTHEENYAARLEAHEANPPKVQKGKKPLKPYEGDMPFFDNGDGTTTFNFKCYGSYEDKKTGETKKIVLGVVDAKGKRIQDVPIIGGGSKVKIRFSLVPYGWSAVAGASVKLQLEGVMLVELATFGGGEDDWADEAVEGGYEADEPRSRKPQEDPEDWSGEEADEGEAEEDDDF

SEQ ID NO: 45 - protein, 239 aa, synthetic construct
  REGION 1..239: note = Synthetic: Single-stranded DNA binding protein from Escherichia phage vB_EcoP_GA2A (with N-terminal His6 tag)
MHHHHHHGSAKEQLKTFTTPVAGIVEPYAWLNKADTKFNERGEHKVNLTFDLSDPKVRMMIDVLQKIHDDAYAKALADHEKNPPQVQRGKKPIEPREGDMPWIENGDGTVTMKFKCFASYLKDGKSEPIVLRFYDTDAKLIRDVPNIGAGSKLKVKFKVLPFKWNAATGASVKLQLESCLLVELKEWKGDGAGGDGGWGDDEDLGTGYKASTDGDFGSDDFGEDADGGDDSASGGDYDF

SEQ ID NO: 46 - protein, 239 aa, synthetic construct
  REGION 1..239: note = Synthetic: Single-stranded DNA binding protein from Yersinia phage vB_YenP_AP10 (with N-terminal His6 tag)
MHHHHHHGSAIKRKVFTTPKGTSEPYAYIAKPDFGGQGFENPRGTYKVSLTLPNDSKTQAMIDAIVACHEEDYARRLEEHEANPPKVVKGKKPLQPYTGDLPFFDNGDGTTTFNFKAHASYEDKKTGESKKINLSVVDSQGKRINDVPLIAGGSELKVKFSMVPYGWTNVAGASVKLQLEGVMIISLATFGGESDDWGDEIEEGGYTADNSRSRNPEQEEGHDFGSDDESGDDDESGDF

SEQ ID NO: 47 - protein, 240 aa, synthetic construct
  REGION 1..240: note = Synthetic: Single-stranded DNA binding protein from Salmonella phage phiSG-JL2 (with N-terminal His6 tag)
MHHHHHHGSAGFKKKIYTSGLGTAEPYAYLSKPDYGNEERGFGNPRGVYKVDLTLSNKDPRCQAMVDEIVKTHEEAYAAAVEEFEANPPQVQRGKKPLKPYEGDMPFFDNGDGTTTFKFKCYASFQDKKTKETKHINLVVVDSKGKKIQEVPIIGGGSKLKVKYSLVPYKWNTAVGASVKLQLESVMLVELATFGGGGEDEWADEVEDGGYTASESRQSRDEQEWQEDEHEETPDDNEDF

SEQ ID NO: 48 - protein, 242 aa, synthetic construct
  REGION 1..242: note = Synthetic: Single-stranded DNA binding protein from Salmonella phage Vi06 (with N-terminal His6 tag)
MHHHHHHGSAKKIYTSALGTAEPYAYITKPDYGNEERGFGNPRGVYKVDLTIPNKDPRCQRMVDEIVKCHEEAYAAAVEEYEANPPAVARGKKPLRPYEGDMPFFDNGDGTTTFKFKAYASYQDKKTKETKHINMVVVDSKGKKIEDVPIIGGGSKLKVKYSLIPYKWNTAVGASVKLQLESVMLVELATFGGGEDDWADEVEENGYVASGSAKASKPRDEESWSQDSDEDAYGEGDSDGDF

SEQ ID NO: 49 - protein, 240 aa, synthetic construct
  REGION 1..240: note = Synthetic: Single-stranded DNA binding protein from Enterobacteria phage 13a (with N-terminal His6 tag)
MHHHHHHGSAKKIFTSALGTAEPYAYIAKPDYGNEERGFGNPRGVYKVDLTIPNKDPRCQRMVDEIVKCHEEAYAAAVEEYEANPPAVARGKKPLKPYEGDMPFFDNGDGTTTFKFKCYASFQDKKTKETKHINLVVVDSKGKKMEDVPIIGGGSKLKVKYSLVPYKWNTAVGASVKLQLESVMLVELATFGGGEDDWADEVEENGYVASGSAKASKPRDEESWDEDDEGSYEEDSDGDF

SEQ ID NO: 50 - protein, 240 aa, synthetic construct
  REGION 1..240: note = Synthetic: Single-stranded DNA binding protein from Escherichia phage 64795_ec1 (with N-terminal His6 tag)
MHHHHHHGSAKKIFTSALGTAEPYAYIGKPDYGNEERGFGNPRGVYKVDLTIPNKDPRCQRMVDEIVKCHEEAYAAAVEEYEANPPAVARGKKPLKPYEGDMPFFDNGDGTTTFKFKCYASFQDKKTKETKHINLVVVDSKGKKMEDVPIIGGGSKLKVKYSLVPYKWNTAVGASVKLQLESVMLVELATFGGGEDDWADEVEENGYVASGSAKASKPRDEESWDEDDEESDEVDEDGDF

SEQ ID NO: 51 - protein, 367 aa, synthetic construct
  REGION 1..367: note = Synthetic: DNA ligase from Enterobacteria phage T7 (with C-terminal His6 tag)
MMNIKTNPFKAVSFVESAIKKALDNAGYLIAEIKYDGVRGNICVDNTANSYWLSRVSKTIPALEHLNGFDVRWKRLLNDDRCFYKDGFMLDGELMVKGVDFNTGSGLLRTKWTDTKNQEFHEELFVEPIRKKDKVPFKLHTGHLHIKLYAILPLHIVESGEDCDVMTLLMQEHVKNMLPLLQEYFPEIEWQAAESYEVYDMVELQQLYEQKRAEGHEGLIVKDPMCIYKRGKKSGWWKMKPENEADGIIQGLVWGTKGLANEGKVIGFEVLLESGRLVNATNISRALMDEFTETVKEATLSQWGFFSPYGIGDNDACTINPYDGWACQISYMEETPDGSLRHPSFVMFRGTEDNPQEKMGSHHHHHH

SEQ ID NO: 52 - protein, 332 aa, synthetic construct
  REGION 1..332: note = Synthetic: DNA ligase from Vibrio phage VP3 (with C-terminal His6 tag)
MTNFKINPFKAVSFKESAIQKVMELGGVIAAVKYDGLRCHLVITPTVDIQGKPAARMVAISRTDKAIPALRELFISDEHKILLGQLLEESLYPEGLVIDGEVMVKGVDFNTGSGMLRRKEAIHVSKLEYKVYGVLPLSAIKASPDTEIEVPYCVMYEQVKVLMHQLWELLPEMDWKQEETYEVYDMASLEELFKKVRAEGHEGLIIKDPMTNWKRGKKTGYWKMKPEGEIDGTVVGVNWGTVGLANEGKVIGFQVLLENGVVVDANGITQEQMEEYTNLVYKTGHDDCFNGRPVQVKYMEKTPKGSLRHPSFQRWRDLEGAGGSGSHHHHHH

SEQ ID NO: 53 - protein, 348 aa, synthetic construct
  REGION 1..348: note = Synthetic: DNA ligase from Stenotrophomonas phage IME15 (with C-terminal His6 tag)
MMNIKTNPYKAVSFNEKAVEKALKRSHYLIADIKYDGVRGNTLVKPSQGACWLSRVSKPIPAIEHLNDGAARWDSLLKDNRCMFPTGFMLDGELMVDGVDFNTGSGLLRTKWLDSKNQEFHNEFLPRNVKKSEKIPFRLDAGLLKVVIYAVLPIEVAESGDDYPVKNLLMQEHVKNMLPLLRAHFPEISWQAAESHEVYDMTELSALYEMKREEGHEGLIVKDPSDIYKRGKKTGWWKMKPENEADGVIQGLVWGTKGLANEGKVIGFEVLLESGRVVNACNIAQSLMNEFTAAVETHGEDYYNGWTCQISYMEETPDGSLRHPSFVMFRGTEAEPTEKMGSHHHHHH

SEQ ID NO: 54 - protein, 316 aa, synthetic construct
  REGION 1..316: note = Synthetic: DNA ligase from Caulobacter phage Percy (with C-terminal His6 tag)
MSKDYIAGKFVEVRKLAKTTGLGKTLHKVSLDEAFEALGKEYEAQSKHDGCSGVVFLHADDTKPTTVLSRTGEECLSMQMQGRLLRDDFDTVVSKYGPLVLLTEAWWPGKGEFAEISGAFRKKSEQRPRLMFVVSDILTLEEFNAGKTSVPYRARMDRGRGVQPKTAAWTWTTRHPPGTYGDPRDLCAKYVRGGGFDGVVLVDLGAGWVKEEDKSGRKIKVKNELSFALRVKEVKVATGEKTGRDVYTLVVGFDGRDLGVGSGVPHKVEDVPKVGDIVEIVAMDYSSDGLLREPRYKGIRADVIKEDRGSHHHHHH

SEQ ID NO: 55 - protein, 351 aa, synthetic construct
  REGION 1..351: note = Synthetic: DNA ligase from Morganella phage vB_MmoP_MP2 (with C-terminal His6 tag)
MLNIKTNPYKAVSFNENAIMKAAEKAGSLIADIKYDGVRLNLVAKHTGGFFLSRTSKVIPALEHLCGYSERWENFLADERQIYKGGIMIDGEAMIKGVDFNTGSGILRTKWLSKKNVKFTTQPDYEIPRGMGKVPFELDPQNLKVVVYAVLPLDVVESGEDHDVMNCVMPYHVDSVIELLVEYFPEIDWVRAESHDVYSMMQLDKLYEFVRERGDEGLVVKDPLDIYKRGKKTGWWKMKPENEADGTIVGLVWGTKGKANEGKVIGFEVLLENGRVVNATNISQALMEEFTLNVKTQELNGDTEAYIGWACQITYMEETPDGSLRHPSFDKFRGTEDNPKEKMGSHHHHHH

SEQ ID NO: 56 - protein, 349 aa, synthetic construct
  REGION 1..349: note = Synthetic: DNA ligase from Klebsiella phage KP32 (with C-terminal His6 tag)
MMNIKTNPFKAVSFVRSAIEKALETSGYLIADTKHDGVRGNICVDNTANAAWLSRVSKTVPALEHLNGFDMRWQKLLKDDRWIFPDGFMLDSELMVKGVDFNTGSGLLRTKWLKSKNYEFSTYAIEKEWDNPKGKIPFHLNSADLKVVLYDIIPLDIIESGDDYNVMTLLRLEHVKVALPVLQDHFPEVEWCLSESHEVYDMDELDALYRQKREEGHEGLVVKDPRGIYKRGKKSGWFKCKPNDEADGIIQGVNWGTPGLANEGKVIGFSVLLETGRLVDANNISRALMDEFTANVKEHGEEYYNGWSCQIGYMEETKDGSLRHPSFTCFRGTEDNPSEKIGSHHHHHH

SEQ ID NO: 57 - protein, 344 aa, synthetic construct
  REGION 1..344: note = Synthetic: DNA ligase from Vibrio phage N4 (with C-terminal His6 tag)
MTNFKINPFKAVSFKESAIQKVMELGGVIAAVKYDGLRCHLVITPTADIQGKPAARMVAISRTDKAIPALRELFISAEHKILLGQLLEESLYPEGLVIDGEVMVKGVDFNTGSGMLRRKEAIHVSKLAYKVYGVLPLNALKASPDAEIEVPYCVMYEQVKVLMHQLWELLPEMDWKQEETYEVYDMASLEELFKKVRAEGHEGLIIKDPMTNWKRGKKTGYWKMKPEGEIDGTVVGVNWGTVGLANEGKVIGFQVLLENGVVVDANGITQEQMEEYTNSIKGYGQYHYCDQYTAEEAVFHGRPVQVKYMEKTPKGSLRHPSFQRWRDLEGAEGVKAGSHHHHHH

SEQ ID NO: 58 - protein, 346 aa, synthetic construct
  REGION 1..346: note = Synthetic: DNA ligase from Salmonella phage phiSG-JL2 (with C-terminal His6 tag)
MNIFKTNPFKAVSFVESAVKKALETSGYLIADIKYDGVRGNIVVDNVAEAAWLSRVSKFIPALEHLNGFDKRWQQLMNDDRCIFPDGFMLDGELMVKGVDFNTGSGLLRTKWLKKNNFMFDRGGVEPLKGSKVAFELDPKLLSVRLYAVLPIHIAESGEDYDVQNLLMPYHVEAMRSLLVEYFPEIEWLIAETYEVYDMDSLTELYEEKRAEGHEGLIVKDPQGIYKRGKKSGWWKLKPECEADGIIQGVNWGTEGLANEGKVIGFSVLLETGRLVDANNISRALMDEFTANVKAHGEDFYNGWACQVNYMEETPDGSLRHPSFEKFRGTEDNPQEKMGSHHHHHH

SEQ ID NO: 59 - protein, 364 aa, synthetic construct
  REGION 1..364: note = Synthetic: DNA ligase from Escherichia phage 64795_ec1 (with C-terminal His6 tag)
MNIKTSPFKAVSFVESAVKKALDNAGYLIVDCKYDGVRGNICVDNTANSCWLSRVSKTIPALEHLNGFDVRWKSLLNDDRCFYKDGFMLDGELMVKGVDFNTGSGLLRTKWLNKKNFEFDVGGVPPLKGSKVEFALDTKRLSVRLYAILPLHIVESGEDCDVMTLLMQEHVKNMLPLLQEYFPEIEWQAAESYEVYDMVELQQLYEKKRAEGHEGLIVKDPMCIYKRGKKSGWWKMKPENEADGIIQGLVWGTEGLANEGKVIGFEVLLESGRVVNATNISRALMDEITETVKEATLSQWGFFSPYGIGDNDACTINPYDGWSCQISYMEETPDGSLRHPSFVMFRGTEDNPQEKMGSHHHHHH

SEQ ID NO: 60 - protein, 240 aa, synthetic construct
  REGION 1..240: note = Synthetic: Single-stranded DNA binding protein from Enterobacteria phage T7 (with C-terminal His6 tag)
MAKKIFTSALGTAEPYAYIAKPDYGNEERGFGNPRGVYKVDLTIPNKDPRCQRMVDEIVKCHEEAYAAAVEEYEANPPAVARGKKPLKPYEGDMPFFDNGDGTTTFKFKCYASFQDKKTKETKHINLVVVDSKGKKMEDVPIIGGGSKLKVKYSLVPYKWNTAVGASVKLQLESVMLVELATFGGGEDDWADEVEENGYVASGSAKASKPRDEESWDEDDEESEEADEDGDFGSHHHHHH

SEQ ID NO: 61 - protein, 225 aa, synthetic construct
  REGION 1..225: note = Synthetic: Single-stranded DNA binding protein from Delftia phage IME-DE1 (with C-terminal His6 tag)
MADKPTVIVTPQATVFGWVNILKPDTKYNAAGDYKIKVRIPKDTKGLAAQLEKIEEAREKAKAAWLKEPKNKGKRLKEADLPFYEDDEGNIVMSFKSVASWTDAKSGETRYRQIPVFGGAGRIDPKQVPQFGEGSIVRVAYTISAFANAAVGAGASLRIDSVKLIEVKQFSGGGAANHFGDDEEGYVPESGGDDSDPYGNDDTGSDAGGSAGDNDEFGSHHHHHH

SEQ ID NO: 62 - protein, 240 aa, synthetic construct
  REGION 1..240: note = Synthetic: Single-stranded DNA binding protein from Kluyvera phage Kvp1 (with C-terminal His6 tag)
MAFAKRKVFTSARGVAEPYCYLAKPDFGSGDFKNERGVYKVSLTVSNDDPRCQKMIDEIVEAHETDYAARLEEYEANPPKVVKGKKPLKPYVGDMPFMDNEDGTTTFNFKCYGSFTDKKTGENKPIDLAIVDSKGKRIRGERPAISGGSELKIKYTLFPYGWSAVAGASVKLQLDSVMLIKLVEFGGGDDDWSDETEEDGYEADEAQTRKPQNGSGWNEEPEEEEDDEGGDFGSHHHHHH

SEQ ID NO: 63 - protein, 238 aa, synthetic construct
  REGION 1..238: note = Synthetic: Single-stranded DNA binding protein from Stenotrophomonas phage IME15 (with C-terminal His6 tag)
MAKKIFTSALGTAEPYAYLAKPDYGNEERGFGNPRGVYKVDLTIPNKDPRCQRMVDEIVKCHEEAYAAAVEEYEANPPAVARGKKPLKPYEGDLPFFDNGDGTTTFKFKCYASFQDKKTKETKHINLVVVDSKGKKLEEVPIIGGGSKLKVKYSLVPYKWNTAVGASVKLQLESVMLVELATFGGGEDDWADEVEEGGYVASGFARSKPRDEEDWSADTEEESAGDDEDFGSHHHHHH

SEQ ID NO: 64 - protein, 201 aa, synthetic construct
  REGION 1..201: note = Synthetic: Single-stranded DNA binding protein from Synechococcus phage P60 (with C-terminal His6 tag)
MADRNRYVFEATLEGFINVYEDEGKFNNRCFSFKLPKDVIEAAEADREELLEWAKSKAPNPRRCEVALSKWDDEGLVKYSYSEEKPKAIPVFVDTEGSLIDKAVLKDVRKGTKVKLIVQQGPYVFGNKVGTRLLVKGVQIIELASGNGSVDSGDMSAEDVAAIFGTTSGFKQSDPQVRKAAEAEPETTESYDFGSHHHHHH

SEQ ID NO: 65 - protein, 195 aa, synthetic construct
  REGION 1..195: note = Synthetic: Single-stranded DNA binding protein from uncultured phage MedDCM-OCT-S04-C24 (with C-terminal His6 tag)
MPRTIHKTEQPVILEGYQAVLKPSKFGYSLSAIVDGSMVDALETDREESLQWAQGKLKNPKRSVLKPEPWEEVADNQFKVKFSWNEENRPPVVDTEGTPVTDENTPMYSGSRVNLAFYQKPYILKDGVTYGTSLKLVGVQLVSLNSGAGVDTGDMAAEDVAALFGKTEGFKANDPAVTVLPGSDDDFGSHHHHHH

SEQ ID NO: 66 - protein, 247 aa, synthetic construct
  REGION 1..247: note = Synthetic: Single-stranded DNA binding protein from Morganella phage vB_MmoP_MP2 (with C-terminal His6 tag)
MSGIDYRKKVFTTPVGTCEPYAYIRQPDFGGEGFKNPRGTYKVDLTLSKDDAGCQKMMDAIVACHEESYAARVADYEENPPKVQRGKKPLVPYEGDMPFIDNDDGTVTFKFKAYGSFTDKKTGENREIKLLVVDSKGKKLEPVPIIAQGSQLKVKFKMVPYGWTPTVGASVKLQLESIMVVELAEFGGGSDLWGDDDFEDGFSADQNPAAKSRKGDDNEDDEPWDTDGDDEPTDDADDFGSHHHHHH

SEQ ID NO: 67 - protein, 239 aa, synthetic construct
  REGION 1..239: note = Synthetic: Single-stranded DNA binding protein from Klebsiella phage K11 (with C-terminal His6 tag)
MAFAKKKIYTTKIGTCEPYAYFNKPDYGGEGFENPRGTYKGYVTFKNEDCQELVDLIVKTHEENYAARLEAHEANPPKVQKGKKPLKPYEGDMPFFDNGDGTTTFNFKCYGSYEDKKTGETKKIVLGVVDAKGKRIQDVPIIGGGSKVKIRFSLVPYGWSAVAGASVKLQLEGVMLVELATFGGGEDDWADEAVEGGYEADESRSRKPQEDPEDWSGEEADEGEAEEDDDFGSHHHHHH

SEQ ID NO: 68 - protein, 239 aa, synthetic construct
  REGION 1..239: note = Synthetic: Single-stranded DNA binding protein from Klebsiella phage KP32 (with C-terminal His6 tag)
MAFAKKKIYTTKIGTCEPYAYFNKPDYGGEGFENPRGTYKGSVTFKNEDCQELVDLIVKTHEENYAARLEAHEANPPKVQKGKKPLKPYEGDMPFFDNGDGTTTFNFKCYGSYEDKKTGETKKIVLGVVDAKGKRIQDVPIIGGGSKVKIRFSLVPYGWSAVAGASVKLQLEGVMLVELATFGGGEDDWADEAVEGGYEADEPRSRKPQEDPEDWSGEEADEGEAEEDDDFGSHHHHHH

SEQ ID NO: 69 - protein, 239 aa, synthetic construct
  REGION 1..239: note = Synthetic: Single-stranded DNA binding protein from Escherichia phage vB_EcoP_GA2A (with C-terminal His6 tag)
MAKEQLKTFTTPVAGIVEPYAWLNKADTKFNERGEHKVNLTFDLSDPKVRMMIDVLQKIHDDAYAKALADHEKNPPQVQRGKKPIEPREGDMPWIENGDGTVTMKFKCFASYLKDGKSEPIVLRFYDTDAKLIRDVPNIGAGSKLKVKFKVLPFKWNAATGASVKLQLESCLLVELKEWKGDGAGGDGGWGDDEDLGTGYKASTDGDFGSDDFGEDADGGDDSASGGDYDFGSHHHHHH

SEQ ID NO: 70 - protein, 239 aa, synthetic construct
  REGION 1..239: note = Synthetic: Single-stranded DNA binding protein from Yersinia phage vB_YenP_AP10 (with C-terminal His6 tag)
MAIKRKVFTTPKGTSEPYAYIAKPDFGGQGFENPRGTYKVSLTLPNDSKTQAMIDAIVACHEEDYARRLEEHEANPPKVVKGKKPLQPYTGDLPFFDNGDGTTTFNFKAHASYEDKKTGESKKINLSVVDSQGKRINDVPLIAGGSELKVKFSMVPYGWTNVAGASVKLQLEGVMIISLATFGGESDDWGDEIEEGGYTADNSRSRNPEQEEGHDFGSDDESGDDDESGDFGSHHHHHH

SEQ ID NO: 71 - protein, 240 aa, synthetic construct
  REGION 1..240: note = Synthetic: Single-stranded DNA binding protein from Salmonella phage phiSG-JL2 (with C-terminal His6 tag)
MAGFKKKIYTSGLGTAEPYAYLSKPDYGNEERGFGNPRGVYKVDLTLSNKDPRCQAMVDEIVKTHEEAYAAAVEEFEANPPQVQRGKKPLKPYEGDMPFFDNGDGTTTFKFKCYASFQDKKTKETKHINLVVVDSKGKKIQEVPIIGGGSKLKVKYSLVPYKWNTAVGASVKLQLESVMLVELATFGGGGEDEWADEVEDGGYTASESRQSRDEQEWQEDEHEETPDDNEDFGSHHHHHH

SEQ ID NO: 72 - protein, 242 aa, synthetic construct
  REGION 1..242: note = Synthetic: Single-stranded DNA binding protein from Salmonella phage Vi06 (with C-terminal His6 tag)
MAKKIYTSALGTAEPYAYITKPDYGNEERGFGNPRGVYKVDLTIPNKDPRCQRMVDEIVKCHEEAYAAAVEEYEANPPAVARGKKPLRPYEGDMPFFDNGDGTTTFKFKAYASYQDKKTKETKHINMVVVDSKGKKIEDVPIIGGGSKLKVKYSLIPYKWNTAVGASVKLQLESVMLVELATFGGGEDDWADEVEENGYVASGSAKASKPRDEESWSQDSDEDAYGEGDSDGDFGSHHHHHH

SEQ ID NO: 73 - protein, 240 aa, synthetic construct
  REGION 1..240: note = Synthetic: Single-stranded DNA binding protein from Enterobacteria phage 13a (with C-terminal His6 tag)
MAKKIFTSALGTAEPYAYIAKPDYGNEERGFGNPRGVYKVDLTIPNKDPRCQRMVDEIVKCHEEAYAAAVEEYEANPPAVARGKKPLKPYEGDMPFFDNGDGTTTFKFKCYASFQDKKTKETKHINLVVVDSKGKKMEDVPIIGGGSKLKVKYSLVPYKWNTAVGASVKLQLESVMLVELATFGGGEDDWADEVEENGYVASGSAKASKPRDEESWDEDDEGSYEEDSDGDFGSHHHHHH

SEQ ID NO: 74 - protein, 240 aa, synthetic construct
  REGION 1..240: note = Synthetic: Single-stranded DNA binding protein from Escherichia phage 64795_ec1 (with C-terminal His6 tag)
MAKKIFTSALGTAEPYAYIGKPDYGNEERGFGNPRGVYKVDLTIPNKDPRCQRMVDEIVKCHEEAYAAAVEEYEANPPAVARGKKPLKPYEGDMPFFDNGDGTTTFKFKCYASFQDKKTKETKHINLVVVDSKGKKMEDVPIIGGGSKLKVKYSLVPYKWNTAVGASVKLQLESVMLVELATFGGGEDDWADEVEENGYVASGSAKASKPRDEESWDEDDEESDEVDEDGDFGSHHHHHH

SEQ ID NO: 75 - protein, 140 aa, synthetic construct
  REGION 1..140: note = Synthetic: AP2, bacteriophage lambda DNA packaging protein (with C-terminal His6 tag)
MTKDELIARLRSLGEQLNRDVSLTGTKEELALRVAELKEELDDTDETAGQDTPLSRENVLTGHENEVGSAQPDTVILDTSELVTVVALVKLHTDALHATRDEPVAFVLPGTAFRVSAGVAAEMTERGLARMQGSHHHHHH

SEQ ID NO: 76 - protein, 92 aa, synthetic construct
  REGION 1..92: note = Synthetic: AP3, bacteriophage T7 hypothetical protein (with N-terminal His6 tag)
MHHHHHHGSLTPINQLLKNPNDIPDVPRATAEYLQVRFNYAYLEASGHIGLMRANGCSEAHILGFIQGLQYASNVIDEIELRKEQLRDDGED

SEQ ID NO: 77 - protein, 249 aa, synthetic construct
  REGION 1..249: note = Synthetic: AP4, bacteriophage T4 deoxynucleoside monophosphate kinase (with C-terminal His6 tag)
MKLIFLSGVKRSGKDTTADFIMSNYSAVKYQLAGPIKDALAYAWGVFAANTDYPCLTRKEFEGIDYDRETNLNLTKLEVITIMEQAFCYLNGKSPIKGVFVFDDEGKESVNFVAFNKITDVINNIEDQWSVRRLMQALGTDLIVNNFDRMYWVKLFALDYLDKFNSGYDYYIVPDTRQDHEMDAARAMGATVIHVVRPGQKSNDTHITEAGLPIRDGDLVITNDGSLEELFSKIKNTLKVLGSHHHHHH

SEQ ID NO: 78 - protein, 891 aa, synthetic construct
  REGION 1..891: note = Synthetic: T7 RNApol (with N-terminal His6 tag)
MHHHHHHGSNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 79 - protein, 885 aa, synthetic construct
  REGION 1..885: note = Synthetic: RNApol137 (with N-terminal His6 tag)
MHHHHHHGSYTSDELYQKQLELEGEMHGYGVTRFDRNNQRAIDSGTPSDTDWNRRLLSNFIEPMVKGINAYKEYYKTKAGRPTVALKYIRQVQPEQAAYIAIKNILDVLGSTTFDANWLVTTIGRRIEDQVRFTKLEEAAPKYVGKVKDSLAKRNSLQYAHQHKVLVATEKKLAENPDRAQLELPRWQEWAEEDCKHVGSLLVNIFEQCILFEGEPVIRKEVQTVRKGTLVFIKPTEKVTKWIHEFREAIGGLAPAYAPCVVPPLDWTSPFTGGFHTEAVSSTLHLAKVRNKRHLRKLTKEQMPAVYKAVNNLQKVRWRISERVLATANTLVELGLPYALPSKDESDWKEKNPCPVPEYLQDLRGEQLKAALTASQWEAFQEWKQLARQNYDEESERVASFREVVRTLGQANRYVGFDAIYFVYTLDFRGRVYCQSSLVSPQGGDLQKALIKFADGMKLGERGEYWFKVHGANEWGWDKKTFDERVALVSEPEFCEMCLDIASDPVTFNDWIKADKPWQFLNWCFEYADFLKHVQSAGNPQDFVSYIPCAMDGSCSGIQHYSAMLRDTVGGTAVNLVNSDKPNDIYGEVCKVTIKELQAIADGSQVYDSKIDPVLAQQLAQEWLRLQPNRSLTKKPVMTLPYGSTQLTCREHVSQWLKDLQKEENKRAKAEFREPMKVHAFGDADSAMPLKFAESLMTSIVWHSIGKVVVAARAGMAYIKAVTSSVAKMNMPLEWTTPTGFIVRQEIYQFTTRQVNTQLLGGTKFVVSTKSKDIDYHRMINSCAPNFVHSMDASHLTLATNYFADAGISSIAVIHDSFGTHAGATDLLRERLRASMVDMYEQHDVITNFLAETEGRLMTAFEHIRVPERGELQLDSINQSTYAFA

SEQ ID NO: 80 - protein, 883 aa, synthetic construct
  REGION 1..883: note = Synthetic: RNApol126 (with N-terminal His6 tag)
MHHHHHHGSDLQEIQLQLENEMFNGGIRRFEADQQRHLQAGNASDTAWNRKLISEFIAPMAQGIQAYKEEYKGKIGRAPRALAFLNCVENEVSAYITMKVVMDMIQTDVTLQSIAMSIADRIEDQVRFSNLEGKAKKYFEKVKASLKASRSKQYAHGHKVMVVAEKNLAEKSEDIDRWIPWGKEELLNIGLTLLEILENSVFFNGEPVFFRTIRTMGYKKTLYFLQTSENIGEWVRAFKDHVAQLSPAYAPCVVPPRPWKSPFNGGFHTEKVASRIRLVKGDREHVRKLTQKQMPKVYKAINALQNTQWQVNKDILNVADEVVRLDLGYGVPHFKPLIDKDNKPANPVPVEFQHLRGRELKEVLSDEQWQAFIHWKGECAKLYTAETKRGSKSASVVRMLGQARKYSMFDAIYFVYAMDSRSRVYAQSSTLSPQSDDLGKSLLRFTEGRSINSVEDLKWFCINGANLWGWDKKTFDVRLNNVLQEDFQEMCRDIASDPLTFTQWVGADAPYQFLAWAFEYANYLDLVDEGRAHEFKTHLPVHQDGSCSGIQHYSAMLKDEVGARAVNLKPSDAPQDIYGEVAKVVIRKNNENMEASEEDFMTSGSMHLQGAVLRAMASSWDSVGITRGLTKKPVMTLPYGSTRITCRESVEDYLITLEEEEVKRAIAENRKANKVHPFVSDKEEGQILERDALNYMTALIWPSISEVVKAPIVAMRMIKQLARYASKRNEGLEYTLPTGFILKQKIMATEMLRVRTMLMGDIRMSLQVDTDVVDETAMSGASAPNFVHGHDASHLILTVCDLVDKGIKSIAVIHDSFGTHADNTVALRDSLRHKMVEMYENTNVLQKLLEEHEERWLVDTGIKVPEQGTFDIREILNSDYVFA

SEQ ID NO: 81 - protein, 891 aa, synthetic construct
  REGION 1..891: note = Synthetic: RNApol157 (Prima III, with N-terminal His6 tag)
MHHHHHHGSANVIKPESHNFSDISAAILPFNVLADSYGEALAAEQLMLEHESYQLGEARFIKAMERQVERGEVSDNAVAKPLLDTLIPALAARITEFIEMKQRGKPHVSKGYFAMIKPESAAFIIVKTTLNILAKEESVPVQRVAMAIGGNIEDEIRFGRIRDEEIKHFKERVKPNLDKRNGFIYKKAYMEAVEAGMQDKGELNSTHEAWEKDVKFHVGIRAIEMLIEATGMVQLERKFKGIPDKDHEALHLAPEYVEKLTNRAHALAGISPMYQPMIVKPKRWTGVQGGGYWAKGRRPLNLIRVGSKRALDRYRQVDMPEVYDAINTIQETAWRINKDVLAVVNNVVTWANCPVEDVPSIDKLALPEKPEDIDNNEESLKKWKKAAAAIYRKEKARQSRRISLEFALSQANKFSKYNEIYFPYNMDWRGRVYAIPMFNPQGNDMVKGLLTFAKKVPVGIDGGYWLAVHGANCAGVDKVSLEDRVKWVNDNEANIIASAEAPLDFTWWAEQDSPFCFLAFCFEWAAYVKAGKKPSFESSLPLAFDGTCSGLQHFSAMLRDEIGGAAVNLLPADKPQDIYGIVAVKVNEVLRDLVISGTEDEMQTLEDKKTGEITERLVLGTRTLAAQWLEHGVTRSVTKRSVMTLAYGSKEYGFADQVFEDTVMPAIDNGKGAMFTEPSQACRFMAKLIWDAVSKTVVAAVEAMQWLQSAAKLVSSEVKDKKSGEILKHAMPVHWTTPNGFPVWSEYCKQEQKRIDCVILGTHRMALTINIRDKKEIDAAKQTSGIAPNFVHSMDASHLQMTVNKCFKVYGIHSFAMIHDSFGCHAGFASKMFRAVRETMVETYEEHDVIQEFYNQFEQQLHESQIEKIPVLPRKGNLELREILKSLYTFS

SEQ ID NO: 82 - DNA, 5123 bp, synthetic construct
  misc_feature 1..5123: note = Synthetic: F2798, T7 RNApol 5kb template
  source 1..5123: mol_type = other DNA
cccgcgaaattaatacgactcactatagggagaccacaacccacaacaaaccccagctgttggggccaggacacccagtgagcccatacttgctctttttgtcttcttcagactgcgccatgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgctggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaaggcagcggatccgaggatgccaagaacatcaagaaaggcccagccccattttaccccttggaggatggcactgctggtgaacagctccacaaagcgatgaagcgctacgccttggtgccgggtaccattgcgtttacagacgcccacatcgaggtgaacataacctatgccgagtacttcgaaatgtcggttcggttggcggaggcaatgaagaggtatggcctgaacaccaaccaccgcattgtcgtgtgttcggagaattccctgcaattctttatgcctgtgcttggtgccttgttcatcggcgttgccgttgcacccgctaatgacatatacaacgagagggagctcctgaactcgatgaacatatcccaaccgacggttgtctttgtctccaagaagggacttcagaagatcctgaatgtgcagaagaagctgccaatcattcagaagatcatcatcatggactctaagaccgactaccagggcttccaatccatgtacaccttcgtgacctcacacttgccacccggattcaacgagtacgacttcgttcctgagtccttcgaccgcgataagaccatagcgctgatcatgaatagctctggcagcaccggactgccaaagggtgtggctcttccgcatagaacagcgtgcgttcgtttcagtcatgcacgggacccgattttcgggaaccagatcatacccgatacggccatcctctcagtggtcccatttcaccacgggttcggcatgtttaccactctggggtacctgatctgcgggtttcgcgtggtcctgatgtaccgttttgaggaggaactgttcctgaggagccttcaggactacaagatccaaagtgccttgctcgtgccaacactcttcagtttcttcgcgaaatccacgctgatcgacaagtatgacctgtcaaacctgcacgagatcgcctctggaggggctcctctcagcaaagaggtgggcgaagcagtggccaaacgctttcatctgcccggaatcaggcaagggtatggcctcacggagactaccagcgcgattctcatcacaccggaaggagatgacaagcctggcgccgtcggtaaagtcgtgcccttcttcgaagcaaaggtggtggatctcgataccggcaagaccctcggcgtcaaccagagaggggaactgtgcgtcagaggtcctatgatcatgagcggatacgtgaacaatcccgaggccactaatgccctgatcgacaaagatgggtggcttcattccggcgacattgcctactgggacgaggacgagcacttcttcatagttgaccggctgaagtctctgatcaagtacaaaggctatcaggtcgctcctgccgaactggagagcattctcctgcagcaccccaacatcttcgatgcaggtgtggcggggcttccggacgacgatgctggagaactgccagcagctgtggtcgttctcgaacatgggaaaacgatgacggagaaagagatcgtggactatgtggccagccaagtgactacagcgaagaagctgaggggcggagtcgtctttgtggacgaggtgcccaaagggctgacaggcaagctcgatgctcgcaagattcgggaaattctcattaaggccaagaaaggcggcaagtcaaagctgtgagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaggatccaac

SEQ ID NO: 83 - DNA, 5119 bp, synthetic construct
  misc_feature 1..5119: note = Synthetic: F2802, RNApol137 5kb template
  source 1..5119: mol_type = other DNA
cttaataattaggcactacaataggagaagctgaaaccacaacaaaccccagctgttggggccaggacacccagtgagcccatacttgctctttttgtcttcttcagactgcgccatgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgctggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaaggcagcggatccgaggatgccaagaacatcaagaaaggcccagccccattttaccccttggaggatggcactgctggtgaacagctccacaaagcgatgaagcgctacgccttggtgccgggtaccattgcgtttacagacgcccacatcgaggtgaacataacctatgccgagtacttcgaaatgtcggttcggttggcggaggcaatgaagaggtatggcctgaacaccaaccaccgcattgtcgtgtgttcggagaattccctgcaattctttatgcctgtgcttggtgccttgttcatcggcgttgccgttgcacccgctaatgacatatacaacgagagggagctcctgaactcgatgaacatatcccaaccgacggttgtctttgtctccaagaagggacttcagaagatcctgaatgtgcagaagaagctgccaatcattcagaagatcatcatcatggactctaagaccgactaccagggcttccaatccatgtacaccttcgtgacctcacacttgccacccggattcaacgagtacgacttcgttcctgagtccttcgaccgcgataagaccatagcgctgatcatgaatagctctggcagcaccggactgccaaagggtgtggctcttccgcatagaacagcgtgcgttcgtttcagtcatgcacgggacccgattttcgggaaccagatcatacccgatacggccatcctctcagtggtcccatttcaccacgggttcggcatgtttaccactctggggtacctgatctgcgggtttcgcgtggtcctgatgtaccgttttgaggaggaactgttcctgaggagccttcaggactacaagatccaaagtgccttgctcgtgccaacactcttcagtttcttcgcgaaatccacgctgatcgacaagtatgacctgtcaaacctgcacgagatcgcctctggaggggctcctctcagcaaagaggtgggcgaagcagtggccaaacgctttcatctgcccggaatcaggcaagggtatggcctcacggagactaccagcgcgattctcatcacaccggaaggagatgacaagcctggcgccgtcggtaaagtcgtgcccttcttcgaagcaaaggtggtggatctcgataccggcaagaccctcggcgtcaaccagagaggggaactgtgcgtcagaggtcctatgatcatgagcggatacgtgaacaatcccgaggccactaatgccctgatcgacaaagatgggtggcttcattccggcgacattgcctactgggacgaggacgagcacttcttcatagttgaccggctgaagtctctgatcaagtacaaaggctatcaggtcgctcctgccgaactggagagcattctcctgcagcaccccaacatcttcgatgcaggtgtggcggggcttccggacgacgatgctggagaactgccagcagctgtggtcgttctcgaacatgggaaaacgatgacggagaaagagatcgtggactatgtggccagccaagtgactacagcgaagaagctgaggggcggagtcgtctttgtggacgaggtgcccaaagggctgacaggcaagctcgatgctcgcaagattcgggaaattctcattaaggccaagaaaggcggcaagtcaaagctgtgagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaggatccaac

SEQ ID NO: 84 - DNA, 5120 bp, synthetic construct
  misc_feature 1..5120: note = Synthetic: F2800, RNApol126 5kb template
  source 1..5120: mol_type = other DNA
tggtcttaattagagaccactatagaagagataaattccacaacaaaccccagctgttggggccaggacacccagtgagcccatacttgctctttttgtcttcttcagactgcgccatgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgctggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaaggcagcggatccgaggatgccaagaacatcaagaaaggcccagccccattttaccccttggaggatggcactgctggtgaacagctccacaaagcgatgaagcgctacgccttggtgccgggtaccattgcgtttacagacgcccacatcgaggtgaacataacctatgccgagtacttcgaaatgtcggttcggttggcggaggcaatgaagaggtatggcctgaacaccaaccaccgcattgtcgtgtgttcggagaattccctgcaattctttatgcctgtgcttggtgccttgttcatcggcgttgccgttgcacccgctaatgacatatacaacgagagggagctcctgaactcgatgaacatatcccaaccgacggttgtctttgtctccaagaagggacttcagaagatcctgaatgtgcagaagaagctgccaatcattcagaagatcatcatcatggactctaagaccgactaccagggcttccaatccatgtacaccttcgtgacctcacacttgccacccggattcaacgagtacgacttcgttcctgagtccttcgaccgcgataagaccatagcgctgatcatgaatagctctggcagcaccggactgccaaagggtgtggctcttccgcatagaacagcgtgcgttcgtttcagtcatgcacgggacccgattttcgggaaccagatcatacccgatacggccatcctctcagtggtcccatttcaccacgggttcggcatgtttaccactctggggtacctgatctgcgggtttcgcgtggtcctgatgtaccgttttgaggaggaactgttcctgaggagccttcaggactacaagatccaaagtgccttgctcgtgccaacactcttcagtttcttcgcgaaatccacgctgatcgacaagtatgacctgtcaaacctgcacgagatcgcctctggaggggctcctctcagcaaagaggtgggcgaagcagtggccaaacgctttcatctgcccggaatcaggcaagggtatggcctcacggagactaccagcgcgattctcatcacaccggaaggagatgacaagcctggcgccgtcggtaaagtcgtgcccttcttcgaagcaaaggtggtggatctcgataccggcaagaccctcggcgtcaaccagagaggggaactgtgcgtcagaggtcctatgatcatgagcggatacgtgaacaatcccgaggccactaatgccctgatcgacaaagatgggtggcttcattccggcgacattgcctactgggacgaggacgagcacttcttcatagttgaccggctgaagtctctgatcaagtacaaaggctatcaggtcgctcctgccgaactggagagcattctcctgcagcaccccaacatcttcgatgcaggtgtggcggggcttccggacgacgatgctggagaactgccagcagctgtggtcgttctcgaacatgggaaaacgatgacggagaaagagatcgtggactatgtggccagccaagtgactacagcgaagaagctgaggggcggagtcgtctttgtggacgaggtgcccaaagggctgacaggcaagctcgatgctcgcaagattcgggaaattctcattaaggccaagaaaggcggcaagtcaaagctgtgagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaggatccaac

SEQ ID NO: 85 - DNA, 5119 bp, synthetic construct
  misc_feature 1..5119: note = Synthetic: F2804, RNApol157 5kb template
  source 1..5119: mol_type = other DNA
ttgattaattaacccacactatagggagagaggggcccacaacaaaccccagctgttggggccaggacacccagtgagcccatacttgctctttttgtcttcttcagactgcgccatgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgctggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaaggcagcggatccgaggatgccaagaacatcaagaaaggcccagccccattttaccccttggaggatggcactgctggtgaacagctccacaaagcgatgaagcgctacgccttggtgccgggtaccattgcgtttacagacgcccacatcgaggtgaacataacctatgccgagtacttcgaaatgtcggttcggttggcggaggcaatgaagaggtatggcctgaacaccaaccaccgcattgtcgtgtgttcggagaattccctgcaattctttatgcctgtgcttggtgccttgttcatcggcgttgccgttgcacccgctaatgacatatacaacgagagggagctcctgaactcgatgaacatatcccaaccgacggttgtctttgtctccaagaagggacttcagaagatcctgaatgtgcagaagaagctgccaatcattcagaagatcatcatcatggactctaagaccgactaccagggcttccaatccatgtacaccttcgtgacctcacacttgccacccggattcaacgagtacgacttcgttcctgagtccttcgaccgcgataagaccatagcgctgatcatgaatagctctggcagcaccggactgccaaagggtgtggctcttccgcatagaacagcgtgcgttcgtttcagtcatgcacgggacccgattttcgggaaccagatcatacccgatacggccatcctctcagtggtcccatttcaccacgggttcggcatgtttaccactctggggtacctgatctgcgggtttcgcgtggtcctgatgtaccgttttgaggaggaactgttcctgaggagccttcaggactacaagatccaaagtgccttgctcgtgccaacactcttcagtttcttcgcgaaatccacgctgatcgacaagtatgacctgtcaaacctgcacgagatcgcctctggaggggctcctctcagcaaagaggtgggcgaagcagtggccaaacgctttcatctgcccggaatcaggcaagggtatggcctcacggagactaccagcgcgattctcatcacaccggaaggagatgacaagcctggcgccgtcggtaaagtcgtgcccttcttcgaagcaaaggtggtggatctcgataccggcaagaccctcggcgtcaaccagagaggggaactgtgcgtcagaggtcctatgatcatgagcggatacgtgaacaatcccgaggccactaatgccctgatcgacaaagatgggtggcttcattccggcgacattgcctactgggacgaggacgagcacttcttcatagttgaccggctgaagtctctgatcaagtacaaaggctatcaggtcgctcctgccgaactggagagcattctcctgcagcaccccaacatcttcgatgcaggtgtggcggggcttccggacgacgatgctggagaactgccagcagctgtggtcgttctcgaacatgggaaaacgatgacggagaaagagatcgtggactatgtggccagccaagtgactacagcgaagaagctgaggggcggagtcgtctttgtggacgaggtgcccaaagggctgacaggcaagctcgatgctcgcaagattcgggaaattctcattaaggccaagaaaggcggcaagtcaaagctgtgagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaggatccaac

SEQ ID NO: 86 - DNA, 13138 bp, synthetic construct
  misc_feature 1..13138: note = Synthetic: PP1709-5, T7 RNApol 10kb template
  source 1..13138: mol_type = other DNA
actcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagaggcgccagcaaccgcacctgtggcgccggtgatgccggccacgatgcgtccggcgtagaggcgcgccaatctagaaaagatctcgatcccgcgaaatcgcgaaattaatacgactcactatagggagaccacaacaaaccccagctgttggggccaggacacccagtgagcccatacttgctctttttgtcttcttcagactgcgccatggagtgtccttcgtgccagcatgtctccaaggaggaaacccccaagttctgcagccagtgcggagagaggctgcctcctgcagcccccatagcagattctgagaacaataactccacaatggcgtcggcctcggagggtgaaatggagtgtgggcaggagctgaaggaggaagggggcccgtgcttgttcccgggctcagacagttggcaagaaaaccccgaggagccctgttccaaagcctcctggaccgtccaagaaggagctacatcagaggttttggtagatgctgctgtagacctcatatccgatgaatgggaagctgctaatgccatacccagcaagagaaggaagcaggatgcagccccgcttgaggccgccagcgtgccttctgcagactgtgagcagagcaaaaagaagaaaaggaagaagaaaaagaaggggaacaagtccgcttcctcagagctggcttccttgcccctttctcctgccagcccctgtcacctgactttgctttcaaacccgtggcctcaggacacagccctgccccacagccaagcccagcagagtggccccactggccagccgagccagcccccaggcacagccaccacgccactggagggtgacggcctctccgcgcccaccgaggttggcgacagccccctgcaggcccaggctttgggagaggcaggagtggccacaggaagtgaggctcagagcagcccgcaattccaggaccacacggaaggggaggaccaggacgcttccatcccctctgggggcagaggcctgtcccaggaggggaccggtccccccacctctgctggtgaaggccattctaggactgaagatgctgcccaggagctcctgttgcctgagtcaaaaggaggcagctctgagcccgggacagaactgcagaccaccgagcaacaggcaggggcctcagcctctatggcagttgatgctgtagctgagccagccaatgcagttaaaggggccgggaaggaaatgaaagagaagacccagagaatgaaacagccaccagcaaccactcctcctttcaaaacacactgccaggaagctgagaccaagaccaaggacgagatggctgctgctgaagaaaaagtcggtaagaatgaacaaggggagcctgaagacctcaagaagccagaggggaagaacagaagtgcagctgctgtgaaaaacgagaaggagcaaaaaaaccaggaagcagatgtccaggaagtgaaggcaagcacgctgagcccgggtggaggagtcaccgtgttcttccacgccatcatctctcttcatttcccattcaatcctgacctccataaagtcttcatcagaggaggagaagaatttggggagtcaaaatgggacagcaatatctgtgagctgcactacaccagagacttgggtcatgaccgcgttcttgttgaaggcattgtctgcatttccaagaagcacctagataaatacattccttacaagtacgtcatttataatggggaatcttttgagtatgagttcatttacaagcaccagcagaagaagggcgagtacgtcaaccgctgtctgttcataaaatcttcacttctgggctcaggagactggcatcagtactatgacatagtttatatgaagcctcatgggagactccagaaagtcatgaaccacatcacagacgggccgaggaaggacctggtgaaggggaagcagattgccgctgcgctcatgctggacagcaccttcagcatcctgcagacctgggacaccatcaacctgaacagcttcttcacccagttcgagcagttttgctttgtcctgcaacagcctatgatttatgaaggacaggcacagctgtggaccgatttgcagtacagggagaaagaggtgaagagatacctgtggcaacatctgaaaaaacacgtggtaccattgccggacggaaaaagcacggactttttgcctgtggactgcccagtgaggagtaaactgaaaacaggcctgattgtcctttttgtagtggaaaaaattgagcttttattagaaggcagcctggactggttgtgtcacctcctaacctcagatgccagctcaccagatgagtttcaccgtgacctaagccacatccttgggatacctcagagctggcggctgtacctggtgaacctgtgccaaagatgcatggacacaaggacgtacacctggctgggcgccctgcctgtcctgcactgctgtatggagctggccccgcggcacaaggatgcctggagacagcctgaggacacctgggccgctctggagggactctccttctcaccgttccgggaacaaatgctagatacgagttccctacttcagtttatgagagagaagcagcatttgctgagcatagacgagcctctcttccggtcctggtttagtctgctacctctgagtcacctggttatgtatatggaaaacttcattgagcacctgggtcgttttcctgctcatatcctggactgtctttcagggatttactaccggcttccgggacttgagcaagtcttgaatacgcaggatgttcaggatgttcagaacgttcagaacattttagaaatgctgttgcgactcctggacacttaccgggacaagattcccgaggaggccttgtcaccatcctacctgactgtgtgtctgaaactgcatgaagccatctgcagcagcacaaagctacttaagttttacgagctgccagccttatctgccgagattgtctgcagaatgattagacttctatctctggtggattctgcaggacagagagatgaaactggaaataattcagtccaaacagtcttccaagggacccttgctgctacgaaaaggtggctccgagaagtttttacaaagaacatgctcacatcttcaggtgcctcattcacatacgtcaaggaaattgaggtctggaggcggctggtggaaatccaattccccgcggagcatggctggaaggagtcgttgctgggagacatggaatggaggctcacaaaggaggaacccctctcccagatcactgcctactgcaatagttgctgggacaccaaaggcttagaggacagtgtggccaagaccttcgagaaatgcatcattgaagccgtgagctcagcctgccagtctcagaccagtatccttcaggggttctcttactctgatttgcggaaatttggcatcgtcttgtctgctgtgatcactaagtcctggcctaggaccgcggacaacttcgatgacattttaaagcatctgctcacgttggcagatgtcaagcacgtcttcagattgtgtggaaccgacgagaaaatactagcaaatgtcacagaggatgccaagaggctgatagctgttgccgactctgtgttgacgaaagttgttggtgacctcctaagtggcacgattttagttggacaactggagctgattataaagcacaagaatcagtttcttgacatctggcaactgagggaaaaaagtctttcaccccaggatgaacaatgtgctgtggaggaagcactggattggagaagggaggaactgttacttctaaagaaagagaaaagatgtgttgatagtctcctgaagatgtgtgggaacgtgaaacatctgatacaagtggactttggagtgcttgcagtaagacactcacaagacctcagcagtaaaagattaaatgacaccgtgacagtgagactgtccacctcctcgaactcgcagagggcaacgcattaccacctgagctcccaggtccaagaaatggctgggaagatagacttgctcagagacagccacatcttccagctcttctggcgggaagccgcagagccgctgagtgagcctaaggaggaccaggaagccgcagagttgctgagtgagcccgaagaagaatcagaaaggcacatccttgagcttgaagaggtgtatgactatttgtatcagccttcttacagaaagttcattaagttgcaccaggatctaaagtcaggagaggtcacccttgcagagattgatgtcatcttcaaggactttgtgaataaatacacggacctggattcagaacttaagatcatgtgcaccgtggaccaccaggaccaaagagattggatcaaggaccgagtcgaacagatcaaggaataccatcacctgcaccaggctgtccacgcagccaaggtcatcttgcaggtcaaagagagcctgggactgaacggtgacttcagtgttctcaacactttactaaattttactgataacttcgacgactttcgccgtgaaacactggaccagatcaaccaggagctcatccaggccaaaaagctgctccaggacatcagcgaggcccggtgcaaggggctgcaggctctgtccctgagaaaggagttcatctgctgggtccgggaggctcttggaggcatcaatgagctgaaggtgtttgtggacctggcctccatctcagcgggggagaatgacattgatgtggaccgggtggcctgcttccatgacgctgtgcagggctacgcatccctgctatttaagctggaccccagcgtggacttcagtgcattcatgaagcatctgaaaaagctgtggaaggctctggataaggaccagtacctgcccaggaaactgtgtgactccgccaggaacttggaatggctgaagactgtgaatgagagtcatgggtctgtggaacgctcatccctgaccctggccacggccatcaaccaaagaggcatctatgtgatccaggcgcccaaaggtggccaaaagatttccccagacacggttctgcacttgatccttcctgagagccctggcagccacgaggagtcacgagagtactctttagaggaggtgaaggagcttttgaacaagttgatgctgatgtctggcaagaaggatcgtaacaacacggaagtggagaggttttcagaggtcttctgcagtgtgcagaggctcagccaggccttcatcgacctgcactctgctgggaatatgctgttcaggacgtggatcgccatggcctactgctcccccaagcagggtgtgtccctccaaatggactttggcttggacctggtgacggagcttaaagaaggtggagatgtcactgagctgctggcagccctctgcaggcagatggagcacttccttgacagctggaagagatttgtgacccagaagcgaatggagcacttttacctgaacttctacacggcagagcagctggtttacctgagcactgagctcaggaagcagcccccgagtgatgccgccctaacgatgctatccttcatcaaaagcaactgcaccctgagggatgtcttaagggcctctgtggggtgtgggagtgaggccgccaggtaccgcatgaggagagtcatggaagagctcccgctgatgctcttatcagagttcagcctggtggacaagctgaggatcatcatggagcagtccatgaggtgccttcctgccttcctgcccgactgcctcgacctagagacccttggccactgtctggctcacctggcagggatgggtgggtctcccgtggagcgttgtctcccgagaggtctgcaggtcggccagcccaacctcgtcgtctgtggccactccgaggtgttgccagccgccctggctgtctacatgcaaaccccaagccagcccctgcccacttacgatgaggtgctgctctgcaccccggcaaccacctttgaggaggtggcactgttgctgcgccgctgcctgaccctgggctccctggggcacaaggtctacagcctgctgttcgcagatcagctgagctacgaggtggcacgccaagcggaggagcttttccacaatctgtgcacgcagcagcaccgagaagactaccagctcgtcatggtctgtgatggggactgggagcactgctacctcccctctgccttcagccagcacaaggtcttcgtcaccccccaggcacccctcgaggccatccaagcctacctggcaggtcactaccgggtcccgaagcagaccctgtcggcggcagccgtgttcaatgaccggctgtgtgttgggatcgtggcctcggagcgagcaggtgttggaaagtctctgtacgtgaagaggttgcacgacaaaatgaagatgcagttaaacgtgaaaaatgtgcctctgaaaacaattcgactgatcgaccctcaggtggatgagagccgagtcctgggcgccctgctgcccttcctggatgcgcagtatcagaaggtccccgtgctctttcacctggacgtgacctcctcagtgcagactggaatttgggtgtttcttttcaagctcctcattttacaatacttaatggatataaatgggaaaatgtggcttcggaacccctgccatttgtatatcgttgaaatcctggaaaggaggacgtcagtgccgtcgaggagctcttcagcgctgcgtacacgtgtaccccagttcagttttcttgacatcttcccaaaagtcacctgcaggcctcccaaagaggtgatagacatggagctgagtgccctgaggagtgacacagagcctgggatggatctgtgggagttctgcagcgaaactttccaaagaccttaccagtatttaagacgattcaatcaaaaccaagacctagacacgtttcagtatcaagaaggctctgtcgaaggcaccccggaggaatgcctccagcatttcctgtttcactgcggggtaataaacccatcctggtcagagcttcggaactttgctcggttcctgaattatcagctcagagattgtgaggcctctctcttctgcaatccgagttttattggcgacacactgaggggcttcaagaagttcgtggtgaccttcatgatctttatggcaagagattttgccacaccatcactccacacctctgaccaaagcccggggaagcacatggtcaccatggatggggttagggaagaagatctagcgcccttctccctccggaagaggtgggagtcggagcctcacccatacgttttcttcaatgacgaccacacaaccatgacattcatcggcttccatctgcagcccaacatcaacggcagtgtcgatgccatcagtcacttgactgggaaggtcatcaagagagacgtcatgaccagggacctgtaccagggcctgctgctccagagggtgcccttcaatgtcgactttgataaactgcccagacacaagaaacttgagaggctctgcctgaccttagggatcccccaggccaccgaccccgacaaaacgtatgagctcacaaccgacaatatgcttaaaatccttgccatcgagatgcggttccggtgtgggatcccggttatcatcatgggagaaactggctgtgggaaaaccaggcttattaaattccttagcgacctgcggcgtggtggtaccaatgctgacaccataaagctggtcaaggtgcacggaggaacaactgcagacatgatctactccagagtcagggaggctgaaaatgtggccttcgccaataaggaccaacatcagttggacaccatcttgttttttgatgaagccaacacaacggaagctataagctgtatcaaagaagtcctgtgtgatcatatggtggatggccagcctctggctgaggactctggcctgcatattatagctgcctgcaatccataccggaagcactctgaggagatgatctgccgtttggagtcagctggtttgggctacagggttagtatggaggagacggccgacaggctgggctccattcctctgaggcagctggtataccgggtccatgctctgcccccgagcctgattcctctggtgtgggactttggacaactgagtgacgttgctgaaaagctctacatccagcagattgtccagagactggttgagtccatcagcctagatgaaaacgggactcgcgtgatcacagaagtcctctgcgcctctcagggtttcatgaggaaaacagaagatgagtgcagctttgtcagcctcagggacgtggagcgctgtgtgaaagttttcaggtggttccacgagcacagcgcgatgctcttagcgcagctgaatgcctttctctccaagtccagcgtcagcaaaaatcacaccgagagagatcccgtcctctggtcgttgatgctggccatcggggtgtgttaccatgcctctttagaaaagaaagactcatatcggaaagccatcgccaggttctttccgaaaccgtatgacgacagcaggctgcttctggatgaaataacacgggcacaggatctttttctggacggcgtacctctgaggaaaaccatcgccaagaacttggccttgaaggagaacgtcttcatgatggtcgtctgcatcgagctgaagattcccctcttcctggtggggaagcccggcagctccaagtctctcgccaagaccatcgtggcagacgccatgcagggcccggctgcctactcagatctcttccgcagcctgaagcaggtccacctggtgtccttccagtgcagcccgcactccaccccacagggcatcatcagcaccttccggcagtgcgcccgctttcagcaggggaaggacctgcagcagtacgtctctgtggtggtgttagatgaggtggggctggcggaagactcacccaaaatgcccctgaagactctgcacccgctgctggaagacggatgcattgaagacgatcccgccccccacaaaaaggtcggcttcgtgggcatctccaactgggcccttgaccctgccaagatgaaccggggcatttttgtgtcacgtggcagccccaacgagacagagctcatagagagcgccaagggcatctgctcctcagacatcctcgtccaggaccgagtccaagggtactttgcgtcctttgccaaagcctacgaaacggtgtgtaagcgccaggacaaggaattcttcgggcttcgtgactactacagcctcatcaaaatggtctttgctgcagcaaaggcttcaaatagaaagccttccccgcaagacattgcacaggctgtccttaggaacttcagtggcaaggatgacatccaagctttggacatctttctggccaatttgcccgaggccaagtgctcagaggaagtcagccccatgcagctgatcaaacagaacatctttgggccttctcagaaggtgccgggtggagagcaggaagatgctgagtcccgctacttactcgtgctgaccaaaaactacgtggcactgcagatcctgcagcagacattcttcgagggggaccagcagccggagattatttttggttctggtttccccaaggaccaagagtacacccagctctgcagaaacatcaatcgtgtgaagatctgcatggaaacaggcaagatggtgttgcttctcaacctgcagaacctctacgagagcctctacgacgcactcaaccagtactacgtccacctcggcggccagaagtacgtggacctcggtctggggacccaccgcgtcaaatgtcgggttcaccccaacttccgcctgattgtcattgaagagaaagacgtcgtgtacaaacactttcccatccccctcattaaccggctggagaagcactatctggatatcaacacggtgctggagaaatggcagaagagcatcgtggaggagctctgtgcgtgggtggagaagttcatcaatgtcaaagcacatcatttccagaagaggcacaaatacagcccctctgacgtcttcatcggctaccactcggacgcctgcgcgtctgtggtgctgcaggtcatagagaggcagggtccccgggccttgacggaggaacttcaccagaaggtgtctgaggaggccaaatcgatcctgctgaactgcgctacgcccgatgccgtggtccggctgagcgcctactcgctgggcgggttcgcagcggagtggctgtcgcaggagtactttcacagacagaggcacaactcctttgcagatttccttcaggcacacctgcacacggcagacctggagcgccacgccatcttcacagagatcaccactttctccaggctgctaacaagtcacgactgtgaaattttagaatcagaggtcacaggcagggctccgaaacccacactcctgtggctgcagcagtttgacaccgagtactcattcctcaaagaagtccgaaactgtttaacgaatacagccaaatgtaaaatcctcatttttcagacagattttgaagatggaatccgtagcgcccagctcattgcctcagctaagtattctgttataaatgaaatcaacaaaatacgagaaaatgaggaccgtatcttcgtctatttcatcacaaaactgtcccgggtgggaagaggaacagcctatgtgggcttccacggagggctgtggcagtctgtccacatcgatgacctccggagatccaccctcatggtttctgatgtgaccagggctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaggatccaacacgtgagtatcaagcggccgctagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagttt

SEQ ID NO: 87 - DNA, 13136 bp, synthetic construct
  misc_feature 1..13136: note = Synthetic: PP1491-32, RNApol137 10kb template
  source 1..13136: mol_type = other DNA
actcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagaggcgccagcaaccgcacctgtggcgccggtgatgccggccacgatgcgtccggcgtagaggcgcgccaatctagaaaagatctcgatcccgcgaaatcttaataattaggcactacaataggagaaccacaacaaaccccagctgttggggccaggacacccagtgagcccatacttgctctttttgtcttcttcagactgcgccatggagtgtccttcgtgccagcatgtctccaaggaggaaacccccaagttctgcagccagtgcggagagaggctgcctcctgcagcccccatagcagattctgagaacaataactccacaatggcgtcggcctcggagggtgaaatggagtgtgggcaggagctgaaggaggaagggggcccgtgcttgttcccgggctcagacagttggcaagaaaaccccgaggagccctgttccaaagcctcctggaccgtccaagaaggagctacatcagaggttttggtagatgctgctgtagacctcatatccgatgaatgggaagctgctaatgccatacccagcaagagaaggaagcaggatgcagccccgcttgaggccgccagcgtgccttctgcagactgtgagcagagcaaaaagaagaaaaggaagaagaaaaagaaggggaacaagtccgcttcctcagagctggcttccttgcccctttctcctgccagcccctgtcacctgactttgctttcaaacccgtggcctcaggacacagccctgccccacagccaagcccagcagagtggccccactggccagccgagccagcccccaggcacagccaccacgccactggagggtgacggcctctccgcgcccaccgaggttggcgacagccccctgcaggcccaggctttgggagaggcaggagtggccacaggaagtgaggctcagagcagcccgcaattccaggaccacacggaaggggaggaccaggacgcttccatcccctctgggggcagaggcctgtcccaggaggggaccggtccccccacctctgctggtgaaggccattctaggactgaagatgctgcccaggagctcctgttgcctgagtcaaaaggaggcagctctgagcccgggacagaactgcagaccaccgagcaacaggcaggggcctcagcctctatggcagttgatgctgtagctgagccagccaatgcagttaaaggggccgggaaggaaatgaaagagaagacccagagaatgaaacagccaccagcaaccactcctcctttcaaaacacactgccaggaagctgagaccaagaccaaggacgagatggctgctgctgaagaaaaagtcggtaagaatgaacaaggggagcctgaagacctcaagaagccagaggggaagaacagaagtgcagctgctgtgaaaaacgagaaggagcaaaaaaaccaggaagcagatgtccaggaagtgaaggcaagcacgctgagcccgggtggaggagtcaccgtgttcttccacgccatcatctctcttcatttcccattcaatcctgacctccataaagtcttcatcagaggaggagaagaatttggggagtcaaaatgggacagcaatatctgtgagctgcactacaccagagacttgggtcatgaccgcgttcttgttgaaggcattgtctgcatttccaagaagcacctagataaatacattccttacaagtacgtcatttataatggggaatcttttgagtatgagttcatttacaagcaccagcagaagaagggcgagtacgtcaaccgctgtctgttcataaaatcttcacttctgggctcaggagactggcatcagtactatgacatagtttatatgaagcctcatgggagactccagaaagtcatgaaccacatcacagacgggccgaggaaggacctggtgaaggggaagcagattgccgctgcgctcatgctggacagcaccttcagcatcctgcagacctgggacaccatcaacctgaacagcttcttcacccagttcgagcagttttgctttgtcctgcaacagcctatgatttatgaaggacaggcacagctgtggaccgatttgcagtacagggagaaagaggtgaagagatacctgtggcaacatctgaaaaaacacgtggtaccattgccggacggaaaaagcacggactttttgcctgtggactgcccagtgaggagtaaactgaaaacaggcctgattgtcctttttgtagtggaaaaaattgagcttttattagaaggcagcctggactggttgtgtcacctcctaacctcagatgccagctcaccagatgagtttcaccgtgacctaagccacatccttgggatacctcagagctggcggctgtacctggtgaacctgtgccaaagatgcatggacacaaggacgtacacctggctgggcgccctgcctgtcctgcactgctgtatggagctggccccgcggcacaaggatgcctggagacagcctgaggacacctgggccgctctggagggactctccttctcaccgttccgggaacaaatgctagatacgagttccctacttcagtttatgagagagaagcagcatttgctgagcatagacgagcctctcttccggtcctggtttagtctgctacctctgagtcacctggttatgtatatggaaaacttcattgagcacctgggtcgttttcctgctcatatcctggactgtctttcagggatttactaccggcttccgggacttgagcaagtcttgaatacgcaggatgttcaggatgttcagaacgttcagaacattttagaaatgctgttgcgactcctggacacttaccgggacaagattcccgaggaggccttgtcaccatcctacctgactgtgtgtctgaaactgcatgaagccatctgcagcagcacaaagctacttaagttttacgagctgccagccttatctgccgagattgtctgcagaatgattagacttctatctctggtggattctgcaggacagagagatgaaactggaaataattcagtccaaacagtcttccaagggacccttgctgctacgaaaaggtggctccgagaagtttttacaaagaacatgctcacatcttcaggtgcctcattcacatacgtcaaggaaattgaggtctggaggcggctggtggaaatccaattccccgcggagcatggctggaaggagtcgttgctgggagacatggaatggaggctcacaaaggaggaacccctctcccagatcactgcctactgcaatagttgctgggacaccaaaggcttagaggacagtgtggccaagaccttcgagaaatgcatcattgaagccgtgagctcagcctgccagtctcagaccagtatccttcaggggttctcttactctgatttgcggaaatttggcatcgtcttgtctgctgtgatcactaagtcctggcctaggaccgcggacaacttcgatgacattttaaagcatctgctcacgttggcagatgtcaagcacgtcttcagattgtgtggaaccgacgagaaaatactagcaaatgtcacagaggatgccaagaggctgatagctgttgccgactctgtgttgacgaaagttgttggtgacctcctaagtggcacgattttagttggacaactggagctgattataaagcacaagaatcagtttcttgacatctggcaactgagggaaaaaagtctttcaccccaggatgaacaatgtgctgtggaggaagcactggattggagaagggaggaactgttacttctaaagaaagagaaaagatgtgttgatagtctcctgaagatgtgtgggaacgtgaaacatctgatacaagtggactttggagtgcttgcagtaagacactcacaagacctcagcagtaaaagattaaatgacaccgtgacagtgagactgtccacctcctcgaactcgcagagggcaacgcattaccacctgagctcccaggtccaagaaatggctgggaagatagacttgctcagagacagccacatcttccagctcttctggcgggaagccgcagagccgctgagtgagcctaaggaggaccaggaagccgcagagttgctgagtgagcccgaagaagaatcagaaaggcacatccttgagcttgaagaggtgtatgactatttgtatcagccttcttacagaaagttcattaagttgcaccaggatctaaagtcaggagaggtcacccttgcagagattgatgtcatcttcaaggactttgtgaataaatacacggacctggattcagaacttaagatcatgtgcaccgtggaccaccaggaccaaagagattggatcaaggaccgagtcgaacagatcaaggaataccatcacctgcaccaggctgtccacgcagccaaggtcatcttgcaggtcaaagagagcctgggactgaacggtgacttcagtgttctcaacactttactaaattttactgataacttcgacgactttcgccgtgaaacactggaccagatcaaccaggagctcatccaggccaaaaagctgctccaggacatcagcgaggcccggtgcaaggggctgcaggctctgtccctgagaaaggagttcatctgctgggtccgggaggctcttggaggcatcaatgagctgaaggtgtttgtggacctggcctccatctcagcgggggagaatgacattgatgtggaccgggtggcctgcttccatgacgctgtgcagggctacgcatccctgctatttaagctggaccccagcgtggacttcagtgcattcatgaagcatctgaaaaagctgtggaaggctctggataaggaccagtacctgcccaggaaactgtgtgactccgccaggaacttggaatggctgaagactgtgaatgagagtcatgggtctgtggaacgctcatccctgaccctggccacggccatcaaccaaagaggcatctatgtgatccaggcgcccaaaggtggccaaaagatttccccagacacggttctgcacttgatccttcctgagagccctggcagccacgaggagtcacgagagtactctttagaggaggtgaaggagcttttgaacaagttgatgctgatgtctggcaagaaggatcgtaacaacacggaagtggagaggttttcagaggtcttctgcagtgtgcagaggctcagccaggccttcatcgacctgcactctgctgggaatatgctgttcaggacgtggatcgccatggcctactgctcccccaagcagggtgtgtccctccaaatggactttggcttggacctggtgacggagcttaaagaaggtggagatgtcactgagctgctggcagccctctgcaggcagatggagcacttccttgacagctggaagagatttgtgacccagaagcgaatggagcacttttacctgaacttctacacggcagagcagctggtttacctgagcactgagctcaggaagcagcccccgagtgatgccgccctaacgatgctatccttcatcaaaagcaactgcaccctgagggatgtcttaagggcctctgtggggtgtgggagtgaggccgccaggtaccgcatgaggagagtcatggaagagctcccgctgatgctcttatcagagttcagcctggtggacaagctgaggatcatcatggagcagtccatgaggtgccttcctgccttcctgcccgactgcctcgacctagagacccttggccactgtctggctcacctggcagggatgggtgggtctcccgtggagcgttgtctcccgagaggtctgcaggtcggccagcccaacctcgtcgtctgtggccactccgaggtgttgccagccgccctggctgtctacatgcaaaccccaagccagcccctgcccacttacgatgaggtgctgctctgcaccccggcaaccacctttgaggaggtggcactgttgctgcgccgctgcctgaccctgggctccctggggcacaaggtctacagcctgctgttcgcagatcagctgagctacgaggtggcacgccaagcggaggagcttttccacaatctgtgcacgcagcagcaccgagaagactaccagctcgtcatggtctgtgatggggactgggagcactgctacctcccctctgccttcagccagcacaaggtcttcgtcaccccccaggcacccctcgaggccatccaagcctacctggcaggtcactaccgggtcccgaagcagaccctgtcggcggcagccgtgttcaatgaccggctgtgtgttgggatcgtggcctcggagcgagcaggtgttggaaagtctctgtacgtgaagaggttgcacgacaaaatgaagatgcagttaaacgtgaaaaatgtgcctctgaaaacaattcgactgatcgaccctcaggtggatgagagccgagtcctgggcgccctgctgcccttcctggatgcgcagtatcagaaggtccccgtgctctttcacctggacgtgacctcctcagtgcagactggaatttgggtgtttcttttcaagctcctcattttacaatacttaatggatataaatgggaaaatgtggcttcggaacccctgccatttgtatatcgttgaaatcctggaaaggaggacgtcagtgccgtcgaggagctcttcagcgctgcgtacacgtgtaccccagttcagttttcttgacatcttcccaaaagtcacctgcaggcctcccaaagaggtgatagacatggagctgagtgccctgaggagtgacacagagcctgggatggatctgtgggagttctgcagcgaaactttccaaagaccttaccagtatttaagacgattcaatcaaaaccaagacctagacacgtttcagtatcaagaaggctctgtcgaaggcaccccggaggaatgcctccagcatttcctgtttcactgcggggtaataaacccatcctggtcagagcttcggaactttgctcggttcctgaattatcagctcagagattgtgaggcctctctcttctgcaatccgagttttattggcgacacactgaggggcttcaagaagttcgtggtgaccttcatgatctttatggcaagagattttgccacaccatcactccacacctctgaccaaagcccggggaagcacatggtcaccatggatggggttagggaagaagatctagcgcccttctccctccggaagaggtgggagtcggagcctcacccatacgttttcttcaatgacgaccacacaaccatgacattcatcggcttccatctgcagcccaacatcaacggcagtgtcgatgccatcagtcacttgactgggaaggtcatcaagagagacgtcatgaccagggacctgtaccagggcctgctgctccagagggtgcccttcaatgtcgactttgataaactgcccagacacaagaaacttgagaggctctgcctgaccttagggatcccccaggccaccgaccccgacaaaacgtatgagctcacaaccgacaatatgcttaaaatccttgccatcgagatgcggttccggtgtgggatcccggttatcatcatgggagaaactggctgtgggaaaaccaggcttattaaattccttagcgacctgcggcgtggtggtaccaatgctgacaccataaagctggtcaaggtgcacggaggaacaactgcagacatgatctactccagagtcagggaggctgaaaatgtggccttcgccaataaggaccaacatcagttggacaccatcttgttttttgatgaagccaacacaacggaagctataagctgtatcaaagaagtcctgtgtgatcatatggtggatggccagcctctggctgaggactctggcctgcatattatagctgcctgcaatccataccggaagcactctgaggagatgatctgccgtttggagtcagctggtttgggctacagggttagtatggaggagacggccgacaggctgggctccattcctctgaggcagctggtataccgggtccatgctctgcccccgagcctgattcctctggtgtgggactttggacaactgagtgacgttgctgaaaagctctacatccagcagattgtccagagactggttgagtccatcagcctagatgaaaacgggactcgcgtgatcacagaagtcctctgcgcctctcagggtttcatgaggaaaacagaagatgagtgcagctttgtcagcctcagggacgtggagcgctgtgtgaaagttttcaggtggttccacgagcacagcgcgatgctcttagcgcagctgaatgcctttctctccaagtccagcgtcagcaaaaatcacaccgagagagatcccgtcctctggtcgttgatgctggccatcggggtgtgttaccatgcctctttagaaaagaaagactcatatcggaaagccatcgccaggttctttccgaaaccgtatgacgacagcaggctgcttctggatgaaataacacgggcacaggatctttttctggacggcgtacctctgaggaaaaccatcgccaagaacttggccttgaaggagaacgtcttcatgatggtcgtctgcatcgagctgaagattcccctcttcctggtggggaagcccggcagctccaagtctctcgccaagaccatcgtggcagacgccatgcagggcccggctgcctactcagatctcttccgcagcctgaagcaggtccacctggtgtccttccagtgcagcccgcactccaccccacagggcatcatcagcaccttccggcagtgcgcccgctttcagcaggggaaggacctgcagcagtacgtctctgtggtggtgttagatgaggtggggctggcggaagactcacccaaaatgcccctgaagactctgcacccgctgctggaagacggatgcattgaagacgatcccgccccccacaaaaaggtcggcttcgtgggcatctccaactgggcccttgaccctgccaagatgaaccggggcatttttgtgtcacgtggcagccccaacgagacagagctcatagagagcgccaagggcatctgctcctcagacatcctcgtccaggaccgagtccaagggtactttgcgtcctttgccaaagcctacgaaacggtgtgtaagcgccaggacaaggaattcttcgggcttcgtgactactacagcctcatcaaaatggtctttgctgcagcaaaggcttcaaatagaaagccttccccgcaagacattgcacaggctgtccttaggaacttcagtggcaaggatgacatccaagctttggacatctttctggccaatttgcccgaggccaagtgctcagaggaagtcagccccatgcagctgatcaaacagaacatctttgggccttctcagaaggtgccgggtggagagcaggaagatgctgagtcccgctacttactcgtgctgaccaaaaactacgtggcactgcagatcctgcagcagacattcttcgagggggaccagcagccggagattatttttggttctggtttccccaaggaccaagagtacacccagctctgcagaaacatcaatcgtgtgaagatctgcatggaaacaggcaagatggtgttgcttctcaacctgcagaacctctacgagagcctctacgacgcactcaaccagtactacgtccacctcggcggccagaagtacgtggacctcggtctggggacccaccgcgtcaaatgtcgggttcaccccaacttccgcctgattgtcattgaagagaaagacgtcgtgtacaaacactttcccatccccctcattaaccggctggagaagcactatctggatatcaacacggtgctggagaaatggcagaagagcatcgtggaggagctctgtgcgtgggtggagaagttcatcaatgtcaaagcacatcatttccagaagaggcacaaatacagcccctctgacgtcttcatcggctaccactcggacgcctgcgcgtctgtggtgctgcaggtcatagagaggcagggtccccgggccttgacggaggaacttcaccagaaggtgtctgaggaggccaaatcgatcctgctgaactgcgctacgcccgatgccgtggtccggctgagcgcctactcgctgggcgggttcgcagcggagtggctgtcgcaggagtactttcacagacagaggcacaactcctttgcagatttccttcaggcacacctgcacacggcagacctggagcgccacgccatcttcacagagatcaccactttctccaggctgctaacaagtcacgactgtgaaattttagaatcagaggtcacaggcagggctccgaaacccacactcctgtggctgcagcagtttgacaccgagtactcattcctcaaagaagtccgaaactgtttaacgaatacagccaaatgtaaaatcctcatttttcagacagattttgaagatggaatccgtagcgcccagctcattgcctcagctaagtattctgttataaatgaaatcaacaaaatacgagaaaatgaggaccgtatcttcgtctatttcatcacaaaactgtcccgggtgggaagaggaacagcctatgtgggcttccacggagggctgtggcagtctgtccacatcgatgacctccggagatccaccctcatggtttctgatgtgaccagggctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaggatccaacacgtgagtatcaagcggccgctagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagttt

(19) United States
(12) Patent Application Publication
(10) Pub. No.: US 2026/0286415 A1
(43) Pub. Date: Sep. 24, 2026

(54) METHODS AND COMPOSITIONS FOR NUCLEIC ACID SYNTHESIS

(71) Applicant: PRIMROSE BIO, INC., San Diego, CA (US)

(72) Inventors: Helge ZIELER, Del Mar, CA (US); Dongxin Karen XU, San Diego, CA (US); Alan GREENER, San Diego, CA (US); Christopher SCHVARCZ, San Diego, CA (US)

(73) Assignee: PRIMROSE BIO, INC., San Diego, CA (US)

(21) Appl. No.: 19/471,970

(22) PCT Filed: Apr. 5, 2024
(86) PCT No.: PCT/US2024/023441

§ 371 Date: Oct. 2, 2025

Related U.S. Application Data
(60) Provisional application No. 63/494,423, filed on Apr. 5, 2023.
(60) Provisional application No. 63/494,422, filed on Apr. 5, 2023.

(51) Int. Cl. C12P 19/34; C07K 14/005; C12N 9/00; C12N 9/12

(52) U.S. Cl. CPC C12P 19/34; C07K 14/005; C12N 9/1229; C12N 9/1247; C12N 9/93; C12N 2795/10222; C12Y 207/04004; C12Y 207/07006; C12Y 605/01001

(57) ABSTRACT

The present disclosure provides compositions and methods for using accessory proteins to increase the yield of full-length RNA, to decrease the amount of double stranded RNA, and to increase the efficiency of RNA produced by in vitro transcription.

### REFERENCE TO ELECTRONIC SEQUENCE LISTING

This application contains a Sequence Listing which has been submitted electronically in .XML format and is hereby incorporated by reference in its entirety. Said .XML copy, created on Apr. 4, 2024, is named “7003-0117PWO1.xml” and is 185,082 bytes in size. The sequence listing contained in this .XML file is part of the specification and is hereby incorporated by reference herein in its entirety.

### BACKGROUND

Development of RNA-based therapeutics and vaccines has dramatically accelerated in the past decade. After the success of the mRNA-based vaccines against COVID-19, a large number of mRNA vaccines and therapeutics are in clinical testing, directed against a variety of diseases including infectious diseases, rare diseases, cancer, allergies and others (Kole 2012, Sahin 2014, Fiedler 2016, Sergeeva 2016, Sullenger 2016, Ulmer 2016, Diken 2017, Grunwitz 2017, Iavarone 2017, Kramps 2017, Pardi 2018, Scheiblhofer 2018, Dolgin 2019, Karimkhanilouyi 2019, Zhang 2019, Chaudhary 2021, Barbier 2022, Hogan 2022, Qin 2022, Rohner 2022). Through variations in its structure and/or different delivery mechanisms, RNAs can be designed to affect both systemic and tissue-specific processes, further broadening its utility. mRNA-based medicines can be designed to 1) Supply wild-type human proteins in an alternative to gene therapy, 2) Up-regulate or down-regulate expression of human genes or 3) Deliver new genetic information for complex therapeutic proteins, such as monoclonal antibodies, which are not normally expressed in the human body. The potential is almost limitless, given the safety and flexibility in the design of these therapies and relatively short production ramp-up of new mRNAs. RNA medicines have the potential to revolutionize human health.

Several dozen biotechnology and pharmaceutical companies are active in developing mRNA for medical uses. The field has attracted billions of dollars in funding as healthcare investors believe that mRNA-based drugs and vaccines represent the most promising area of innovation in 21st century drug development.

Despite this promise, commercial development of RNA-based vaccines and therapeutics has been slowed by the difficulty of manufacturing large quantities of commercially suitably material of uniform sequence and length (Rosa 2021, Whitley 2022). Specific RNA sequences can be efficiently generated by in vitro transcription (IVT) from DNA templates using bacteriophage single-subunit RNA polymerases (RNApols), for example the widely used bacteriophage T7 RNA polymerase (T7 RNApol; Sousa 2003, Nayak 2007, Durniak 2008, Borkotoky 2018). IVT reactions have been successfully developed into large-scale manufacturing processes, for example for the mRNAs used in COVID-19 vaccines. However, these processes are currently limited by: 1) Relatively low specific activity which requires long reaction times or high enzyme doses and results in low yields with DNA templates greater than 5 kb in length; 2) Low efficiencies of incorporating modified nucleotides used to stabilize mRNA (Huang 1997, Majlessi 1998, Layzer 2004, Kraynack 2005, Jackson 2006, Wilson 2006, Ge 2010), and 3) Variable RNA quality due to the presence of mutated, aberrant or truncated transcripts (Martin 1988, Konarska 1989, Cazenave 1994, Triana-Alonso 1995, Arnaud-Barbe 1998) or the presence of double-stranded RNA (dsRNA; Cazenave 1994, Triana-Alonso 1995, Arnaud-Barbe 1998). Variable RNA quality can also require extensive and costly purification of the target mRNA to avoid off-target effects of the active ingredient (Rosa 2021, Whitley 2022).

The presence of double stranded RNA (dsRNA) is the most troublesome RNA quality issue, as dsRNA elicits a strong native immune response (Lengyel 1987, Stark 1998, Majde 2000, Gantier 2007, Mu 2018) and needs to be completely removed from clinical RNA preparations (Baiersdörfer 2019, Rosa 2021, Whitley 2022). T7 RNApol is gradually being replaced by superior RNApols, such as those developed by Primrose Bio (as described in WO2020243026A1), but some of the mRNA manufacturing challenges remain, especially for long DNA templates and GC-rich sequences.

A number of scientific teams have worked in the past few years to develop more efficient mRNA manufacturing enzymes and processes (for example as described in WO2020243026A1). However, even the most efficient RNApols have limitations when used in standard in vitro transcription (IVT) processes containing purified RNApols in the absence of other protein co-factors. When transcribing templates greater than 5 kb in length, even the best RNApols fall significantly short of theoretical yields of full-length mRNAs. Shorter templates, especially those with greater than 50% GC content, can also be difficult to produce. Finally, all IVT reactions are dependent on high concentrations of template DNA and RNApol, the two expensive inputs in IVT reactions.

Clinical-grade mRNA requires uniform, full-length mRNA of high purity and high fidelity, with low levels of double stranded RNA (dsRNA) which cause adverse and harmful innate immune responses in patients and can lower mRNA efficacy (Kariko 2004, Kariko 2011, Ziemniak 2013, Shanmugasundaram 2022, Whitley 2022, Warminski 2023). Clinical-grade mRNA further requires a functional capping structure present on a high proportion at least 90% or more of mRNA molecules. As noted above, commercial development of RNA-based vaccines and therapeutics, as well as RNAs used in agriculture, has been slowed by the difficulty of manufacturing large quantities of commercially suitably material of uniform sequence, length and quality.

Because RNA is inherently unstable, RNA intended for human uses are chemically modified to stabilize the molecule and extend its shelf life and half-life in the human body (Majlessi 1998, Layzer 2004, Kraynack 2005, Jackson 2006, Wilson 2006, Kariko 2008, Ge 2010, Warminski 2023). Through variations in its structure and/or different delivery mechanisms, RNAs can be designed to affect both systemic and tissue-specific processes, further broadening its utility. The simplest way to modify RNA is to incorporate non-native nucleotides into RNA during synthesis, for example nucleotides blocked at their 2′ position, or nucleotides like pseudouridine that contain modified bases. Tremendous progress has also been made in the development of various cap analogs that mimic the 5′ cap structure of natural mammalian mRNA and that enhance translation (Pasquinelli 1995, Stepinski 2001, Cougot 2004, Kariko 2008, Kuhn 2011, Kuhn 2012, Grudzien-Nogalska 2013). Incorporation of cap analogs or modified nucleotides into RNA requires development of better performing enzymes and processes to efficiently incorporate the rapidly evolving modified nucleotides and cap structures being developed for synthetic mRNA, and to streamline the manufacturing process.

The single-subunit RNApols being developed for mRNA manufacturing are of bacteriophage origin, and presumably rely on other proteins during the bacterial infection cycle to transcribe the bacteriophage genome. Although little is known about accessory factors of bacteriophage single-subunit RNApols, the related phage-like single-subunit RNApols found in eukaryotic mitochondria and chloroplasts have been shown to interact with other proteins to facilitate promoter recognition and RNApol activity (Jang 1991, Fisher 1992, Xu 1992, Matsunaga 2004, Amiott 2007, Kühn 2007), making it likely that the bacteriophage RNApols also interact positively with cellular or phage-encoded proteins. We have termed such putative RNApol co-factors “accessory proteins” (APs).

The present disclosure describes the use of bacterial or bacteriophage proteins as RNApol accessory proteins to increase the efficiency of in vitro transcription reactions.

### BRIEF SUMMARY

Described herein are methods and compositions relevant to mRNA synthesis during in vitro transcription processes. Various accessory proteins can be added to in vitro transcription reactions to achieve higher yield of in vitro transcription products with better mRNA quality, and enhanced capping efficiency, lower double-stranded RNA levels and other improvements enabling cost reduction of the mRNA manufacturing process.

### DETAILED DESCRIPTION

As used herein, the terms “comprises,” “comprising,” “includes,” “including,” “has,” “having,” “contains,” “containing,” or any other variation thereof, are intended to cover a non-exclusive inclusion. For example, a composition, a mixture, process, method, article, or apparatus that comprises a list of elements is not necessarily limited to only those elements but may include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

“Accessory protein” as used herein refers to any peptide or protein added to an in vitro transcription reaction to increase reaction efficiency, increase RNA yield, increase RNA quality, lower RNA production cost, or any combinations thereof. Accessory proteins as used herein exclude proteins already commonly added to in vitro transcription reactions such as RNase inhibitors and pyrophosphatases.

“Cap,” “5′ cap,” or “mRNA cap” as used herein refers to specialized nucleotides found at the 5′ ends of mRNAs, such as the 7-methylguanosine cap found in eukaryotic mRNAs or other capping structures found at the 5′ end of natural or synthetic RNAs. mRNAs synthesized in vitro can be capped at their 5′ ends by co-transcriptional incorporation of dinucleotide or trinucleotide cap analogs by RNA polymerase.

“Capping efficiency” as used herein refers to the percentage of RNA synthesized in vitro using a single-subunit RNA polymerase that contains a 5′ cap.

“Cap incorporation efficiency” as used herein refers to the efficiency by which a single-subunit RNA polymerase incorporates a dinucleotide or trinucleotide cap analog into mRNA synthesized in vitro. An RNA polymerase with high cap incorporation efficiency can achieve higher capping efficiency at lower concentrations of dinucleotide or trinucleotide cap analog in the reaction than an RNA polymerase with lower cap incorporation efficiency.

“Complementary nucleotide sequence” as used herein, refers to a sequence in a polynucleotide chain in which all of the bases are able to form base pairs with a sequence of bases in another polynucleotide chain.

“Control elements” as used herein refers to nucleotide sequences located upstream (5′ non-coding sequences), within, or downstream (3′ non-coding sequences) of a coding sequence and which influence the transcription, RNA processing, RNA stability, or translation of the associated coding sequence. Regulatory sequences include but are not limited to promoters, translation leader sequences, introns, polyadenylation recognition sequences, RNA processing site(s), effector binding site(s) and stem-loop structure(s).

“Degenerate Sequences” as used herein are populations of sequences where specific sequence positions differ between different molecules or clones in the population. The sequence differences may be a single nucleotide or multiple nucleotides of any number, examples being 2, 3, 4, 5, 6, 7, 8, 9, 10, 20, 30, 40, 50, 60, 70, 80, 90, 100, 200, 300, 400, 500, 600, 700, 800, 900 or 1000 nucleotides, or any number in between. Sequence differences in an individual degenerate sequence may involve the presence of 2, 3 or 4 different nucleotides in that position within the population of sequences, molecules or clones. Examples of degenerate nucleotides in a specific position of a sequence are: A or C; A or G; A or T; C or G; C or T; G or T; A, C or G; A, C or T; A, G or T; C, G or T; A, C, G or T.

“Expression” as used herein, refers to the transcription and stable accumulation of sense (mRNA) or antisense RNA derived from the nucleic acid disclosed, as well as the accumulation of polypeptide as a product of translation of mRNA.

“Fidelity” as used herein describes the accuracy of a nucleic acid polymerase, reflecting faithful copying of a template nucleic acid into a daughter nucleic acid strand. Fidelity also describes the accuracy by which a nucleic acid sample reflects the sequence of the template nucleic acid from which it was copied. For example, a high fidelity DNA or RNA polymerase makes few errors in copying a DNA strand and results in a DNA or RNA sample that is substantially free of misincorporated nucleotides that change the sequence from that of the template DNA when copied into an RNA or a DNA daughter strand. A high fidelity RNA sample is one that contains few misincorporated nucleotides that change the sequence from that of the template DNA from which the RNA sample was derived.

“Full-length Open Reading Frame” as used herein refers to an open reading frame encoding a full-length protein which extends from its natural initiation codon to its natural final amino-acid coding codon, as expressed in a cell or organism. In cases where a particular open reading frame sequence gives rise to multiple distinct full-length proteins expressed within a cell or an organism, each open reading frame within this sequence, encoding one of the multiple distinct proteins, is considered full-length. In different aspects of the disclosure, a full-length open reading frame is either continuous or interrupted by introns.

“Full-length RNA” or “full-length transcript” as used herein refers to an RNA synthesized from a nucleic acid template that covers the entire length of the nucleic acid template, from the transcription initiation site in a 3′ to 5′ direction along the template strand to the end of the nucleic acid template. An RNA molecule transcribed from a nucleic acid template may be considered full-length if it is substantially full-length, meaning that its length differs from the length of the nucleic acid template by a few or multiple nucleotides at either end, such that the migration of a full-length RNA molecule and the substantially full-length RNA molecule cannot be distinguished using commonly used methods of gel electrophoresis and capillary gel electrophoresis. Full-length RNA can also be alternatively referred to as “target RNA”.

“Gene” as used herein refers to a nucleic acid fragment that is capable of being expressed as a specific protein, optionally including regulatory sequences preceding (5′ non-coding sequences) and following (3′ non-coding sequences) the coding sequence. “Native gene” or “natural gene” refers to a gene as found in nature in its natural host organism, complete with its native control elements, including but not limited to a promoter, terminator, ribosome binding site or other translation promoting sequence, enhancer, and repressor binding sites. “Chimeric gene” refers to any gene that comprises regulatory and coding sequences that are not found together in nature. Accordingly, a chimeric gene may comprise regulatory sequences and coding sequences that are derived from different sources and engineered or synthesized by the hand of man, or regulatory sequences and coding sequences derived from the same source, but arranged in a manner different than that found in nature. Similarly, a “foreign” gene refers to a gene not normally found in the host organism, but that is introduced into the host organism by gene transfer. Foreign genes include native genes inserted into a non-native organism, or chimeric genes. A “transgene” is a gene that has been introduced into the genome by a transformation procedure.

“In-Frame” and “in-frame fusion polynucleotide” or “fusion gene” as used herein refers to the reading frame of codons in an upstream or 5′ polynucleotide or open reading frame (ORF) as being the same as the reading frame of codons in a polynucleotide or ORF placed downstream or 3′ of the upstream polynucleotide or ORF that is fused with the upstream or 5′ polynucleotide or ORF. Such in-frame fusion polynucleotides or fusion genes encode a fusion protein or fusion peptide encoded by both the 5′ polynucleotide and the 3′ polynucleotide. Collections of such in-frame fusion polynucleotides can vary in the percentage of fusion polynucleotides that contain upstream and downstream polynucleotides that are in-frame with respect to one another. The percentage in the total collection is at least 10% and can number 10%, 11%, 12%, 13%, 14%, 15%, 20%, 25%, 30%, 35%, 40%, 45%, 50%, 55%, 60%, 65%, 70%, 75%, 80%, 85%, 90%, 95%, 96%, 97%, 98%, 99%, 100% or any percentage in between, typically in excess of 50%.

“In vitro transcription reaction” or “IVT reaction” as used herein, is a reaction designed to produce RNA by transcribing a nucleic acid template in vitro. In vitro transcription reactions typically contain one or more double-stranded DNA template molecules encoding the RNAs to be transcribed; one or more completely or partially purified RNA polymerases such as single-subunit RNA polymerases; nucleotide triphosphates as substrates for the single-subunit RNA polymerase(s) such as the four canonical ribonucleotide triphosphates ATP, CTP, GTP and TTP; buffers, divalent cations and salts as necessary for the RNApol to be active. IVT reactions can also contain additional enzymes such as a pyrophosphatase that degrades pyrophosphate released by the RNA polymerase during RNA synthesis. The nucleic acid template contains a promoter sequence recognized by the RNApol and where the RNApol binds to initiate the transcript.

“Integrity of a nucleic acid” or “RNA integrity” as used herein, refers to the degree to which a collection of nucleic acid molecules have the expected length. For example, RNA molecules transcribed from a linear double-stranded DNA template that measures 2000 base pairs between the transcription start site and the end of the template (measured along the template strand and including the transcription start site) are expected to have a length of 2000 nucleotides. When measured by gel electrophoresis or capillary gel electrophoresis, such RNA molecules may range in size from 250 nucleotides to 2000 nucleotides. If, as measured by gel electrophoresis or capillary gel electrophoresis, half of the RNA molecules have the expected length of 2000 nucleotides and the other half are shorter, then the integrity of this RNA sample is 50%, or stated differently the sample has RNA integrity of 50%. The portion of the RNA molecules which, as measured by gel electrophoresis or capillary gel electrophoresis, have a length of approximately 2000 base pairs corresponds to full-length and substantially full-length RNA molecules.

“Iterate” or “Iterative” as used herein refers to applying a method or procedure repeatedly to a material or sample. Typically, the processed, altered, or modified material or sample produced from each round of processing, alteration, or modification is then used as the starting material for the next round of processing, alteration, or modification. Iterative selection refers to a selection process that iterates or repeats the selection two or more times, using the survivors of one round of selection as starting material for the subsequent rounds.

“Library” as used herein refers to a collection of genes or polynucleotide sequences that are different from each other and that are cloned into a vector for propagation of the sequences. In different libraries, the sequences differ by sequence content, origin, source organism, length, structure, association with other sequences, and/or any other property of a polynucleotide sequence. For example, a library of amino acid repeat fusion genes is generated by cloning a starting open reading frame (ORF) collection that contains multiple different ORFs encoded by the E. coli genome into a bacterial cloning and expression vector that contains a promoter, a sequence encoding an amino acid repeat oriented in a manner that this sequence will be joined directly and in-frame to the ORFs, a terminator, a plasmid backbone and an antibiotic resistance gene. The starting ORF collection can contain any number of ORFs that number 5 or greater, for example 5, 6, 7, 8, 9, 10, 11, 12, 13, 14, 15, 16, 17, 18, 19, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000 or greater, or any number in between. In a specific aspect of the disclosure, the ORF collection used to generate the library contains a sufficient number of ORFs to give a high likelihood of encoding a specific desirable property of E. coli, for example 50% or more of the ORFs encoded by the E. coli genome, or 2074 or more ORFs when using the annotation of the E. coli strain MG1655 genome annotation prepared by the University of Wisconsin, Madison which lists a total of 4148 ORFs.

“Linker sequence” as used herein refers to a polynucleotide sequence or polypeptide sequence separating two polynucleotides or polypeptides in a fusion polynucleotide or fusion polypeptide. For example, a fusion polynucleotide contains two or more open reading frames (ORFs) that are separated by a linker sequence, which encodes a peptide which separates the two parts of the polypeptide that results from expression and translation of the fusion polynucleotide. A linker can also separate an epitope tag from a protein or enzyme. Linker sequences can have diverse length and/or sequence composition.

“Mutation” as used herein refers to an alteration in the nucleic acid sequence of a nucleic acid sequence, gene or the genome of an organism. Mutations include but are not limited to single nucleotide substitutions or point mutations; substitutions of multiple nucleotides; deletions; insertions; sequence duplications; copy-number changes (amplifications or deletions); translocations; chromosomal duplications or deletions; chromosomal rearrangements; genome duplications; or changes in ploidy. Mutations can be subdivided into deleterious mutations which reduce the fitness or productivity of an organism, the function of a gene or the activity of the protein or enzyme encoded by a gene; beneficial mutations which improve the fitness or productivity of an organism, the function of a gene or the activity and other desirable qualities of the protein or enzyme encoded by a gene; or neutral mutations which do not measurably impact the qualities of an organism, gene or encoded protein or enzyme.

“Non-homologous” as used herein refers to sequence identity at the nucleotide level of less than 50%.

“Nucleic acid” as used herein refers to biopolymers, consisting of nucleotides joined to each other via phosphodiester linkages or phosphorothioate linkages. Nucleic acid can be used interchangeably with polynucleotide.

“Nucleic acid polymerase” as used herein refers to an enzyme that catalyzes the polymerization of a nucleic acid using nucleotide triphosphates and unblocked nucleic acids as substrates and sequentially adds single nucleotides to the 3′ end of the unblocked nucleic acid. Nucleic acid polymerases as described in the scientific literature typically fall into the classes of DNA polymerases and RNA polymerases, with DNA polymerases capable of polymerizing DNA and RNA polymerases capable of polymerizing RNA. However, specific enzymes may have the dual ability to catalyze the synthesis of both DNA and RNA. For example, a DNA polymerase may have the ability to add ribonucleotides to the 3′ end of a DNA or RNA molecule, and an RNA polymerase may have the ability to add deoxyribonucleotides to the 3′ end of a DNA or RNA molecule.

“Nucleotides” as used herein refers to the monomer building blocks of nucleic acids, made of three components: a 5-carbon sugar, a phosphate group, and a nitrogenous base. The two main classes of nucleic acids are deoxyribonucleic acid (DNA) and ribonucleic acid (RNA). If the sugar is ribose, the nucleic acid is RNA; if the sugar is the ribose derivative deoxyribose, the nucleic acid is DNA.

“Nucleotide triphosphates” as used herein refers to any of the ribonucleotide triphosphates ATP, CTP, GTP, ITP, UTP and XTP, etc. used in RNA synthesis, or any of the deoxyribonucleotide triphosphates dATP, dCTP, dGTP, dITP, dTTP and dXTP, etc. used in DNA synthesis, or any modified nucleotides or nucleotide analogs, derivatives or variants thereof, including derivatives containing phosphorothioate linkages, modifications of the ribose sugar or modifications of the bases. Mixtures of the four canonical nucleotide triphosphates used in DNA synthesis (dATP, dCTP, dGTP, and dTTP) are denoted by the shorthand “dNTP” and Mixtures of the four canonical nucleotide triphosphates used in RNA synthesis (ATP, CTP, GTP, and UTP) are denoted by the shorthand “NTP”.

“Open Reading Frame (ORF)” as used herein refers to any sequence of nucleotides in a nucleic acid that encodes a protein or peptide as a string of codons in a specific reading frame. Within this specific reading frame, an ORF can contain any codon specifying an amino acid, but does not contain a stop codon. The ORFs in the starting collection need not start or end with any particular amino acid. In different aspects of the disclosure, an ORF is either continuous or is interrupted by one or more introns.

“Operably linked” as used herein refers to the association of nucleic acid sequences on a single nucleic acid fragment so that the function of one is affected by the other. For example, a promoter is operably linked with a coding sequence when it is capable of effecting the expression of that coding sequence (i.e., that the coding sequence is under the transcriptional control of the promoter). Coding sequences can be operably linked to regulatory sequences in sense or antisense orientation.

“Peptide bond” as used herein refers to a covalent bond between a first amino acid and a second amino acid in which the alpha-amino group of the first amino acid is bonded to the alpha-carboxyl group of the second amino acid.

“Percentage of sequence identity” as used herein refers to the degree of identity between any given query sequence, e.g. SEQ ID NO: 102, and a subject sequence. A subject sequence typically has a length that is from about 80 percent to 200 percent of the length of the query sequence, e.g., 80, 82, 85, 87, 89, 90, 93, 95, 97, 99, 100, 105, 110, 115, or 120, 130, 140, 150, 160, 170, 180, 190 or 200 percent of the length of the query sequence. A percent identity for any subject nucleic acid or polypeptide relative to a query nucleic acid or polypeptide is determined as follows. A query sequence (e.g. a nucleic acid or amino acid sequence) is aligned to one or more subject nucleic acid or amino acid sequences using the computer program ClustalW (version 1.83, default parameters), which allows alignments of nucleic acid or protein sequences to be carried out across their entire length (global alignment, Chenna 2003).

ClustalW calculates the best match between a query and one or more subject sequences, and aligns them so that identities, similarities and differences can be determined. Gaps of one or more residues can be inserted into a query sequence, a subject sequence, or both, to maximize sequence alignments. For fast pairwise alignment of nucleic acid sequences, the following default parameters are used: word size: 2; window size: 4; scoring method: percentage; number of top diagonals: 4; and gap penalty: 5. For multiple alignment of nucleic acid sequences, the following parameters are used: gap opening penalty: 10.0; gap extension penalty: 5.0; and weight transitions: yes. For fast pairwise alignment of protein sequences, the following parameters are used: word size: 1; window size: 5; scoring method: percentage; number of top diagonals: 5; gap penalty: 3. For multiple alignment of protein sequences, the following parameters are used: weight matrix: blosum; gap opening penalty: 10.0; gap extension penalty: 0.05; hydrophilic gaps: on; hydrophilic residues: Gly, Pro, Ser, Asn, Asp, Gln, Glu, Arg, and Lys; residue-specific gap penalties: on. The ClustalW output is a sequence alignment that reflects the relationship between sequences. ClustalW can be run, for example, at the Baylor College of Medicine Search Launcher website and at the European Bioinformatics Institute website on the World Wide Web (ebi.ac.uk/clustalw).

To determine a percent identity of a subject or nucleic acid or amino acid sequence to a query sequence, the sequences are aligned using Clustal W, the number of identical matches in the alignment is divided by the query length, and the result is multiplied by 100. It is noted that the percent identity value can be rounded to the nearest tenth. For example, 78.11, 78.12, 78.13, and 78.14 are rounded down to 78.1, while 78.15, 78.16, 78.17, 78.18, and 78.19 are rounded up to 78.2. Sequence identity can be 5%, 6%, 7%, 8%, 9%, 10%, 11%, 12%, 13%, 14%, 15%, 16%, 17%, 18%, 19%, 20%, 30%, 40%, 50%, 60%, 70%, 75%, 80%, 85%, 90%, 91%, 92%, 93%, 94%, 95%, 96%, 97%, 98%, 99%, and any percentage value in between.

“Plasmid” or “vector” as used herein refer to genetic elements used for carrying genes which are not present in an unmodified or wild type cell or organism. Plasmids typically replicate extrachromosomally as autonomous episomal genetic elements, while vectors can either integrate into the genome or can be maintained extrachromosomally as linear or circular DNA fragments. Plasmids and vectors can be linear or circular, and can consist of single- and/or double-stranded DNA or RNA that is derived from any source. Plasmids and vectors often contain a number of nucleotide sequences from different sources which have been joined or recombined into a unique construction which is useful for introducing polynucleotide sequences into a cell or an organism and expressing genes within an organism. The sequences present on a plasmid or on a vector include but are not limited to: autonomously replicating sequences; centromere sequences; sequences homologous to a genome that facilitate integration; origins of replication; control sequences such as promoters or terminators; open reading frames; selectable marker genes such as antibiotic resistance genes; visible marker genes such as genes encoding fluorescent proteins; restriction endonuclease recognition sites; recombination sites; and/or sequences with no apparent or known function. The sequences within a plasmid or vector can be derived from any source or multiple sources. Plasmids and vectors can contain a number of nucleotide sequences that have been joined or recombined into a unique construction which is useful for introducing specific polynucleotide sequences such as protein-coding genes into a cell or an organism.

“Polypeptide” or “protein” as used herein refers to a polymer composed of a plurality of amino acid monomers joined by peptide bonds. The polymer comprises 10 or more monomers, including 10, 11, 12, 13, 14, 15, 16, 17, 18, 19, 20, 30, 40, 50, 60, 70, 80, 90, 100, 200, 300, 400, 500, 600, 700, 800, 900, 1000, 2000, 3000, 4000, 5000, 10000, 20000, 30000, 40000, 50000, 60000, 70000, 80000, 90000, 100000, 200000, 300000, 400000, 500000, 600000, 700000, 800000, 900000, 1000000, or any length in between. A preferred polypeptide or protein of the disclosure is a single-subunit RNA polymerase.

“Promoter” as used herein refers to a DNA sequence capable of controlling the expression of a coding sequence or functional RNA. In general, a coding sequence is located 3′ to a promoter sequence. In different aspects, promoters are derived in their entirety from a native gene, or are composed of different elements derived from different promoters found in nature, or even comprise synthetic DNA segments. It is understood by those skilled in the art that different promoters direct the expression of a gene in different tissues or cell types, or at different stages of development, or in response to different environmental or physiological conditions. Promoters which cause a gene to be expressed in most cell types at most times are commonly referred to as “constitutive promoters”. It is further recognized that since in most cases the exact boundaries of regulatory sequences have not been completely defined, DNA fragments of different lengths may have identical promoter activity.

“Random” or “Randomized” as used herein, means made or chosen without method or conscious decision.

“Reverse transcription-quantitative polymerase chain reaction” or “RT-qPCR” as used herein refers to a method used to quantitate the amount of a certain RNA species or RNA sequence, for example to analyze the transcriptional activity of an RNA polymerase or to characterize the activity of a promoter or other transcriptional control sequence.

“RNA polymerase,” “RNApol,” or “RNAP” as used herein refers to an enzyme that synthesizes a single-stranded RNA molecule from a nucleic acid template, usually double-stranded DNA.

“RNA quality” as used herein refers to the purity of RNA obtained in an in vitro transcription reaction. High RNA quality can mean high RNA integrity, high capping efficiency, low levels of double-stranded RNA, low levels of short, truncated RNAs, low levels of other undesirable side products other than the full-length RNA, high fidelity, high and/or uniform polyA tail length, or any combinations thereof. Low RNA quality can mean low RNA integrity, low capping efficiency, high levels of double-stranded RNA, high levels of short, truncated RNAs, high levels of other undesirable side products, low RNA fidelity, low and/or non-uniform polyA tail length, or any combinations thereof. High RNA quality typically results in high rates of translation of the RNA into the functional or active protein encoded by the RNA.

“Sequence” as used herein in a biological context, implies the sequence of nucleotides in a nucleic acid or the sequence of amino acids in a protein. As used herein, the term “sequence” has a meaning dependent on the context in which the term is used. For example, when used in the context suggesting nucleic acids such as genome sequences, gene sequences or ORFs, then sequence refers to a nucleotide sequence. In a context suggesting proteins or polypeptides, such as the proteome, proteins or enzymes, sequence refers to an amino acid sequence.

“Single-subunit RNA polymerase” as used herein, refers to an enzyme with DNA-dependent RNA polymerase activity capable of synthesizing RNA from a DNA template in vitro in a pure form, without the presence or addition of any other proteins or peptides into the reaction.

“Transformed” as used herein refers to genetic modification by introduction of a polynucleotide sequence.

“Transformation” as used herein refers to the transfer of a nucleic acid fragment into a host organism, resulting in genetically stable inheritance. Host organisms containing the transformed nucleic acid fragments are referred to as “transgenic” or “recombinant” or “transformed” organisms.

“Transformed Organism” as used herein refers to an organism that has been genetically altered by introduction of a polynucleotide sequence into the organism's genome.

“Unfavorable conditions” as used herein implies any part of the growth condition, physical or chemical, that results in slower growth than under normal growth conditions, or that reduces the viability of cells compared to normal growth conditions.

“Variant nucleic acids” as used herein refers to mutated or altered versions of nucleic acid sequences. A variant nucleic acid may have point mutations, insertions, deletions, inversions, rearrangements or combinations thereof compared to the parental or reference sequence that it is derived from or related to. Sequences within a variant nucleic acid that contain mutations, insertions, deletions, inversions, rearrangements or combinations thereof compared to a reference or parental sequence that the variant nucleic acid is related to or derived from, may be of any length, including 1, 2, 3, 4, 5, 6, 7, 8, 9, 10, 11, 12, 13, 14, 15, 16, 17, 18, 19, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000 nucleotides or more, or any number in between. A variant nucleic acid may contain a single change (single mutation, insertion, etc.) compared to a reference or parental sequence, or multiple changes. A variant nucleic acid may have uniform sequences represented within a sample, where all molecules in a nucleic acid sample have the same sequence, or diverse sequences where a sample comprises nucleic acid molecules of different sequence. Variant nucleic acids comprising nucleic acid molecules of different sequence may differ from each other in sequence positions anywhere in the nucleic acid. Variant nucleic acids comprising nucleic acid molecules of different sequence may differ from each other in a particular region of the sequence, or have differences scattered over the entire length of the sequence, or combinations thereof. Variant nucleic acids can contain degenerate or randomized positions, where a specific sequence or region has been replaced by a stretch of degenerate nucleotides. Randomized or degenerate positions within variant nucleic acids may involve adjacent nucleotides or non-adjacent nucleotides separated by nucleotides of a specific or fixed sequence. Variant nucleic acids are frequently employed in biotechnology to create variability within a sequence of interest (coding sequence or non-coding sequence) from which new nucleic acids with specific qualities of interest (for example higher efficiency of an encoded enzyme) can be isolated.

Different RNA polymerases differ in their ability to synthesize RNA. RNA synthesis by an RNA polymerase can also be influenced by the reaction components of the in vitro transcription reaction. For example, the ability of a single-subunit RNA polymerase to synthesize a uniform population of RNA molecules in vitro decreases with the length of the DNA molecule used as a template for the RNA polymerase. Certain RNA polymerases are capable of synthesizing RNAs of 100 nucleotides, 500 nucleotides, 1 kb, 2 kb, 3 kb, 4 kb, 5 kb, 6 kb, 7 kb, 8 kb, 9 kb, 10 kb, 11 kb, 12 kb, 13 kb, 14 kb, 15 kb, 16 kb, 17 kb, 18 kb, 19 kb, 20 kb, 21 kb, 22 kb, 23 kb, 24 kb, 25 kb, 26 kb, 27 kb, 28 kb, 29 kb, 30 kb, 40 kb, 50 kb in length or longer or shorter, or any length in between. Certain RNA polymerases have higher processivity than others, or an improved ability to synthesize full-length RNAs from longer templates (for example, templates encoding mRNAs longer than 5 kb), and are capable of synthesizing highly uniform RNAs greater than 1 kb in length or longer. The composition of the in vitro transcription reaction, including the concentrations of the main reaction components (double-stranded DNA template molecules encoding the RNAs to be transcribed; single-subunit RNA polymerases; nucleotide triphosphates as monomers for RNA synthesis; buffers, divalent cations and salts as necessary for the RNApol to be active and accessory enzymes such as pyrophosphatase).

Single-subunit RNA polymerases and/or in vitro transcription reactions also differ in their ability to utilize non-natural nucleotides and incorporate these into the RNA molecule. Examples of such non-natural nucleotides are 2′-O-methyl NTPs, 2′-fluoro NTPs, pseudouridine-5′-triphosphate and N1-methylpseudouridine-5′-Triphosphate. The 2′ hydroxyl of ribonucleotides has frequently been targeted for modification because this group is primarily responsible for the low stability of RNA under basic conditions. Various modifications at the 2′ position of nucleotides have been tested for increasing RNA stability. However, some single-subunit RNA polymerases incorporate such modified nucleotides inefficiently. Alternatively, RNA molecules containing such modified nucleotides may exhibit a high rate of sequence errors. Specific single-subunit RNA polymerases among the ones described in this disclosure are able to incorporate modified nucleotides efficiently without compromising sequence fidelity.

Single-subunit RNA polymerases and/or in vitro transcription reactions differ in their RNA yield based on the nucleotides added to an in vitro transcription reaction. For example, a 1 ml in vitro transcription reaction containing 5 mM of each of the four nucleotide triphosphates ATP, CTP, GTP and TTP can yield up to about 6.43 mg of RNA (the ‘theoretical yield’) assuming equal representation of each of the nucleotides in the DNA template and complete incorporation of nucleotide triphosphates into RNA in the reaction. An RNA polymerase that synthesizes 2.5 mg of RNA in such a reaction has a yield of 38.9%. Higher-yielding RNA polymerases and/or in vitro transcription reactions are of value as they maximize the amount of RNA product made from a specific amount of nucleotide triphosphates added to the reaction. For example, the accessory proteins disclosed herein increase the transcript yield generated by T7 RNA polymerase or by RNApol137.

Yield enhancement during in vitro transcription can mean increasing the absolute amount of RNA synthesized in the reaction with all reaction components being the same (approaching the theoretical yield) or increasing RNA yield while reducing the reaction concentrations of the double-stranded DNA template or of the RNA polymerase. Such improved reactions are said to increase RNA yield on template or on RNA polymerase. Accessory proteins added to an in vitro transcription reaction can increase the RNA yield on template or increase the RNA yield on RNA polymerase or increase the RNA yield on any other reaction component that is expensive or otherwise limiting and for which it may benefit the producer of the RNA to lower the concentration of said component.

RNA yield as described above can be expressed as total RNA yield, which includes all RNA molecules synthesized in the reaction, regardless of their length, or full-length RNA yield, which includes only the full-length and substantially full-length RNA molecules synthesized in the reaction. For example, an RNA polymerase or in vitro transcription reaction may produce a measurably higher RNA yield than full-length RNA yield. Addition of certain accessory proteins to in vitro transcription reaction may change either total RNA yield or full-length RNA yield.

Single-subunit RNA polymerases and/or in vitro transcription reactions differ in the amount of double-stranded RNA made in a reaction. Double-stranded RNA is a frequent and undesirable side product of in vitro transcription reactions (Arnaud-Barbe 1998, Mu 2018, Gholamalipour 2018), and its reduction or elimination reduces the cost of synthesizing pharmaceutical-grade RNA.

Single-subunit RNA polymerases and/or in vitro transcription reactions differ in the amount of short or truncated RNAs made in a reaction. Short or truncated RNAs can be any RNAs that are not full-length and are frequent and undesirable side products of in vitro transcription reactions. They represent aborted or incomplete transcription products of a template (Martin 1988); their reduction or elimination reduces the cost of synthesizing pharmaceutical-grade RNA.

Single-subunit RNA polymerases and/or in vitro transcription reactions differ in their ability to incorporate a 5′-cap such as the 7-methylguanosine cap found in eukaryotic mRNAs or other capping structures into the 5′ end of RNAs. mRNAs used in biotechnology can be capped by incorporating a specialized dinucleotide or trinucleotide cap analog into the 5′ end of the mRNA. Co-transcriptional incorporation of dinucleotide or trinucleotide caps is catalyzed by the RNA polymerase during transcription initiation. The composition of in vitro transcription reactions as disclosed herein can be varied to increase the rate of cap incorporation and cap utilization.

Single-subunit RNA polymerases and/or in vitro transcription reactions differ in their temperature specificity or reaction speed at varying temperatures, both of which are important parameters in RNA synthesis. Lower reaction temperatures such as between 10° C. and 20° C. can stabilize the RNA. However, T7 RNA polymerase has very low activity at such temperatures. It is therefore of value to identify RNA polymerases active at low temperatures.

Single-subunit RNA polymerases and/or in vitro transcription reactions differ in their overall reaction speed, irrespective of temperatures. Faster enzymes are typically more desirable because shorter reaction times reduce RNA degradation.

Single-subunit RNA polymerases and/or in vitro transcription reactions differ in their fidelity. High-fidelity RNA polymerases will produce RNAs that faithfully encode the sequence of the template DNA used to synthesize the RNA and faithfully encode a protein of interest. High-fidelity RNA polymerases therefore have higher utility when synthesizing RNAs for therapeutic or vaccine applications.

Current in vitro transcription reactions (Henderson 2021) comprise double-stranded DNA template molecules encoding the RNAs to be transcribed; single-subunit RNA polymerases; nucleotide triphosphates as monomers for RNA synthesis; buffers, divalent cations, reducing agents, polyamines, and salts as necessary for the RNApol to be active or for other protein reagents in the reaction to be active, such as RNAse inhibitors and pyrophosphatase. In vitro transcription reactions can also contain cap analogs to be added to the 5′ end of the mRNA (Henderson 2021).

Because the single-subunit RNA polymerases in use in in vitro transcription reactions evolved in a cellular context to be active during a bacteriophage infection of a bacterial cell or to be active within a mitochondrion, chloroplast or other organelle, it is likely that they co-evolved with other proteins that modify or alter the RNA polymerase activity. The activity of single-subunit RNA polymerases may be altered in the presence of any proteins involved in nucleic acid metabolism and nucleic acid related cellular functions, including but not limited to: nucleic acid binding proteins, nucleic acid polymerases, nucleic acid ligases, single-stranded DNA binding proteins, single-stranded RNA binding proteins, nucleases, nucleoside kinases, nucleoside monophosphate kinases, nucleoside diphosphate kinases, bacteriophage or viral capsid proteins, bacteriophage or viral proteins involved in nucleic acid packaging, proteins playing a role in DNA or RNA replication, proteins playing a role in transcription, proteins playing a role in RNA splicing, histones, histone-like proteins, hypothetical or uncharacterized bacteriophage or viral proteins, bacteriophage or viral proteins of unknown function, or charged proteins capable of interacting with nucleic acids.

Such proteins that alter the activity of a single-subunit RNA polymerases, termed accessory proteins in the context of this disclosure, can alter any aspect of the RNA polymerase's activity, including but not limited to increased RNA yield (total or full-length RNA), increased RNA yield on template RNA yield per molecule of template DNA), increased RNA yield on RNA polymerase (RNA yield per molecule of RNA polymerase), increased RNA integrity, increased RNA polymerase processivity, decreased synthesis by the RNA polymerase of undesirable reaction products other than full-length RNA such as double-stranded RNA or short, truncated RNAs, increased incorporation of non-natural nucleotides into the RNA, increased transcription fidelity, increased co-transcriptional capping efficiency or increased cap utilization efficiency.

The natural functions of accessory proteins that alter the in vitro activity of a single-subunit RNA polymerase can be any of the functions of proteins altering the activity of nucleic acid polymerases and single-subunit RNA polymerases listed above.

In some embodiments, one or more accessory proteins added to an in vitro transcription reaction may improve RNA polymerase activity and/or increase RNA yields by 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 60%, 70%, 80%, 90%, 100%, 200%, 300%, 400%, 500%, 600%, 700%, 800%, 900%, 1000% or more, or any number in between, compared to not adding an accessory protein to an in vitro transcription reaction. Increases in RNA yield can be reflected in total RNA yield of full-length RNA yield, or both. Increases in RNA yield can be of unmodified RNA, or RNA modified by incorporation of one or more modified nucleotides or nucleotide analogs.

In some embodiments, one or more accessory proteins added to an in vitro transcription reaction may increase RNA integrity by 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 60%, 70%, 80%, 90%, 100%, or any number in between, compared to not adding an accessory protein to an in vitro transcription reaction.

In some embodiments, one or more accessory proteins added to an in vitro transcription reaction may reduce the amount of double-stranded RNA formed in the reaction, or reduce the amount of other undesirable side products such as short or truncated RNAs, by 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 60%, 70%, 80%, 90%, 100%, or any number in between, compared to not adding an accessory protein to an in vitro transcription reaction.

In some embodiments, one or more accessory proteins added to an in vitro transcription reaction may increase the capping efficiency by 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 60%, 70%, 80%, 90%, 100%, 200%, 300%, 400%, 500%, 600%, 700%, 800%, 900%, 1000% or more, or any number in between, compared to not adding an accessory protein to an in vitro transcription reaction.

In some embodiments, one or more accessory proteins added to an in vitro transcription reaction may increase the cap incorporation efficiency by 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 60%, 70%, 80%, 90%, 100%, 200%, 300%, 400%, 500%, 600%, 700%, 800%, 900%, 1000% or more, or any number in between, compared to not adding an accessory protein to an in vitro transcription reaction.

Measurements of RNA polymerase activity, or quality metrics of RNA synthesized in in vitro transcription reactions, are generated using standardized methods and assays. RNA yield is measured by purification of the RNA after the in vitro transcription reaction, followed by spectroscopic or fluorescence measurement of RNA concentrations (Gandhi 2020, Hadi 2023). RNA yield and integrity are measured by gel electrophoresis (Henderson 2021, Tu 2024) and quantitation of the fluorescence intensity of RNA bands using ImageJ or related software (Schindelin 2012, Schneider 2012, Rueden 2017, Poveda 2019) or other methods for quantitating fluorescent band intensities. RNA yield and integrity are also determined with capillary electrophoresis-based methods (Poveda 2019, Warzak 2023) using commercially available instruments such as the Fragment Analyzer manufactured by Agilent Corporation (Santa Clara, CA, USA). Capillary electrophoresis methods are also suitable for measuring other RNA qualities such as polyA tail length and uniformity (Di Grandi 2023, Tu 2024). RNA integrity can also be addressed using reverse transcription-qPCR (Poveda 2019, Di Grandi 2023). Double-stranded RNA present in RNA synthesized in in vitro transcription reactions is quantitated using dot blots or ELISA assays based on monoclonal antibodies that specifically bind double-stranded RNA (Aramburu 1991, Karikó 2011, Baiersdörfer 2019), such as the J2 IgG2a monoclonal antibody and the and the IgG2a K1 and IgM K2 monoclonal antibodies (Schönborn 1991) and the 9D5 monoclonal antibody (Son 2015). Double-stranded RNA levels can also be determined using reverse transcription-qPCR (Poveda 2019, Di Grandi 2023). Capping efficiency and cap incorporation efficiency can be measured with a variety of methods including gel electrophoresis, fluorescence spectroscopy (when using fluorescently labeled cap analogs), nanopore sequencing and liquid chromatography-mass spectrometry (Tu 2024). RNA polymerase and RNA fidelity are addressed by a variety of sequencing methods, including RNA sequencing following reverse transcription and nanopore sequencing (Gholamalipour 2018, Poveda 2019, Gunter 2023). RNA quality is also measured by in vitro translation followed by enzymatic assays (for RNAs encoding enzymes whose activity can be determined in vitro) and cell-based assays (Poveda 2019).

Accessory proteins can be discovered by expressing and testing individual proteins encoded by bacteriophages that encode single-subunit RNA polymerases, proteins encoded by bacteriophages that do not encode single-subunit RNA polymerases, proteins encoded by bacteria that may or may not be the host for such bacteriophages, proteins encoded by non-bacterial organisms that also encode single-subunit RNA polymerases (Kühn 2007, Paratkar 2011, Arnold 2012, Deshpande 2012, Velazquez 2012, Bestwick 2013, Gualberto 2014, Börner 2015, Pfannschmidt 2015, Posse 2017), or proteins encoded by non-bacterial organisms that do not encode single-subunit RNA polymerases.

Accessory protein candidates can be added to in vitro transcription reactions to assess their effect on RNA yield, RNA quality or other variables related to the in vitro transcription reaction products.

Accessory proteins can be full-length proteins encoded in bacteriophage, bacterial, prokaryotic, eukaryotic or archaeal genomes, or fragments of such proteins.

Accessory proteins discovered in the genomes of bacteriophages, bacteria or other organisms that affect RNA yield, RNA quality or other variables related to in vitro transcription reaction products may lead to the discovery of other, related accessory proteins present in the same bacteriophage, bacterial or other organisms' genomes or in genomes of different bacteriophages or bacteria or other organisms by virtue of sequence similarity to accessory proteins already discovered.

Accessory proteins may alter the in vitro transcription reaction by interacting with and changing the activity of the RNA polymerase or by interacting with the double-stranded DNA template, or by interacting with the RNA transcript, or a combination thereof.

Accessory proteins made be produced recombinantly using methods known in the art and described herein. Accessory proteins can be added to an in vitro transcription reaction in purified form or in crude form. Crude form implies that the protein is released from its production organism or production system without elimination of other, contaminating proteins or with incomplete elimination of other, contaminating proteins. When in crude or purified form, an accessory protein can have different levels of purity, expressed as the percentage of total protein represented by the accessory protein, including 1%, 2%, 3%, 4%, 5%, 6%, 7%, 8%, 9%, 10%, 20%, 30%, 40%, 50%, 60%, 70%, 80%, 90%, 100%, or any percentage in between.

Accessory proteins may include an affinity tag at the N- or C-terminus, such as a histidine tag or His6 tag, to aid in purification. The sequence listing of this invention contains different versions of multiple proteins as detailed in Table 1 below.

TABLE 1
Accessory protein sequences listed as native (unmodified)
proteins, N-terminally His6-tagged proteins and
C-terminally His6-tagged proteins.

| SEQ ID NO | SEQ ID NO | SEQ ID NO |
|---|---|---|
| of native | of accessory | of accessory |
| (unmodified) | protein | protein |
| accessory | with N-terminal | with C-terminal |
| protein | His6 tag | His6 tag |
| 1 | 27 | 51 |
| 2 | 28 | 52 |
| 3 | 29 | 53 |
| 4 | 30 | 54 |
| 5 | 31 | 55 |
| 6 | 32 | 56 |
| 7 | 33 | 57 |
| 8 | 34 | 58 |
| 9 | 35 | 59 |
| 10 | 36 | 60 |
| 11 | 37 | 61 |
| 12 | 38 | 62 |
| 13 | 39 | 63 |
| 14 | 40 | 64 |
| 15 | 41 | 65 |
| 16 | 42 | 66 |
| 17 | 43 | 67 |
| 18 | 44 | 68 |
| 19 | 45 | 69 |
| 20 | 46 | 70 |
| 21 | 47 | 71 |
| 22 | 48 | 72 |
| 23 | 49 | 73 |
| 24 | 50 | 74 |

Accessory proteins can be peptides or proteins of any length, including 5, 6, 7, 8, 9, 10, 11, 12, 13, 14, 15, 16, 17, 18, 19, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000 amino acids or more, or any number in between.

Accessory proteins can be added to in vitro transcription reactions either singly or in combination with other accessory proteins. An in vitro transcription reaction may therefore contain any number of different accessory proteins, including 1, 2, 3, 4, 5, 6, 7, 8, 9, 10, 11, 12, 13, 14, 15, 16, 17, 18, 19, 20, 30, 40, 50, 60, 70, 80, 90, 100 or more different proteins.

Accessory proteins can be added to in vitro transcription reactions in any amount or stoichiometry relative to the RNA polymerase present in the in vitro transcription reaction. For example, the molar ratio of the RNA polymerase and the accessory protein can be 1:1/1,000,000, 1:1/100,000, 1:1/10,000, 1:1/1,000, 1:1/900, 1:1/800, 1:1/700, 1:1/600, 1:1/500, 1:1/400, 1:1/300, 1:1/200, 1:1/100, 1:1/90, 1:1/80, 1:1/70, 1:1/60, 1:1/50, 1:1/40, 1:1/30, 1:1/20, 1:1/10, 1:1/9, 1:1/8, 1:1/7, 1:1/6, 1:1/10, 1:1/5, 1:1/4, 1:1/3, 1:1/2, 1:1, 1:2, 1:3, 1:4, 1:5, 1:6, 1:7, 1:8, 1:9, 1:10, 1:20, 1:30, 1:40, 1:50, 1:60, 1:70, 1:80, 1:90, 1:100, 1:200, 1:300, 1:400, 1:500, 1:600, 1:700, 1:800, 1:900, 1:1,000, 1:10,000, 1:100,000, 1:1,000,000 or any ratio higher, lower or in between, where ratios with a fraction following the colon denote an excess of RNA polymerase and ratios with an integer greater than 1 following the colon denoting excess of accessory protein. Similarly, the accessory protein can be added in various molar ratios relative to the nucleic acid template present in the in vitro transcription reaction. The molar ratio of the template nucleic acid (expressed as a concentration per base pair of the template) and the accessory protein can be 1:1/1,000,000, 1:1/100,000, 1:1/10,000, 1:1/1,000, 1:1/900, 1:1/800, 1:1/700, 1:1/600, 1:1/500, 1:1/400, 1:1/300, 1:1/200, 1:1/100, 1:1/90, 1:1/80, 1:1/70, 1:1/60, 1:1/50, 1:1/40, 1:1/30, 1:1/20, 1:1/10, 1:1/9, 1:1/8, 1:1/7, 1:1/6, 1:1/10, 1:1/5, 1:1/4, 1:1/3, 1:1/2, 1:1, 1:2, 1:3, 1:4, 1:5, 1:6, 1:7, 1:8, 1:9, 1:10, 1:20, 1:30, 1:40, 1:50, 1:60, 1:70, 1:80, 1:90, 1:100, 1:200, 1:300, 1:400, 1:500, 1:600, 1:700, 1:800, 1:900, 1:1,000, 1:10,000, 1:100,000, 1:1,000,000 or any ratio higher, lower or in between, where ratios with a fraction following the colon denote an excess of template base pairs relative to the accessory protein and ratios with an integer greater than 1 following the colon denoting excess of accessory protein relative to base pairs of the template.

When observing improved RNA yields, RNA quality or other improvements in the performance of in vitro transcription reactions containing accessory proteins, it is possible to maximize the effect of the accessory protein by titrating or varying the amount of accessory protein added to the reaction.

### EXAMPLES

### Example 1: Increased RNA Yields Obtained from In Vitro Transcription Reactions Containing Accessory Proteins

The RNA polymerases (SEQ ID NO: 78-79) and accessory proteins (SEQ ID NO: 27, 36, and 75-77) were expressed and purified for use in in vitro transcription (IVT) reactions. To produce each purified protein, an expression plasmid containing the gene was transformed into E. coli strain BL21 and a single colony picked for cultivation and protein expression. The bacterial cells were grown in LB medium at 30° C. to log phase culture and induced by addition of L-arabinose. After 18 hours of incubation at 16° C., the cultures were harvested by centrifugation and the collected E. coli cells lysed. Expressed proteins were purified with nickel affinity chromatography using MAGNEHIS™ Ni Particles (Promega catalog #V8565) according to the manufacturer's instructions. Protein was eluted with imidazole solution, concentrated with AMICON® Ultra-centrifugal filter sold by Millipore (Darmstadt, Germany), and transferred to a storage buffer composed of 50 mM Tris pH 8.0, 75 mM NaCl, 0.05 mM EDTA, 10 mM 2-mercaptoethanol, and 50% glycerol. The purified proteins were stored at −20° C. until experimentation.

In vitro transcription (IVT) reactions were run using either T7 RNA polymerase (SEQ ID NO:78) or RNA polymerase 137 (RNApol137, SEQ ID NO:79) using double-stranded DNA templates incorporating the specific promoters of either polymerase. The 5 kb templates (SEQ ID NO: 82 and SEQ ID NO: 83) were generated by PCR amplification. The 10 kb templates (SEQ ID NO: 86 and SEQ ID NO: 87) were generated by digestion (linearization) of purified plasmid DNA with the restriction enzyme NotI.

20 μL IVT reactions were run in quadruplicate and contained 40 mM Tris-HCl pH 8.0, 6 mM MgCl2, 10 mM dithiothreitol, 2 mM spermidine, 5 mM ATP, 5 mM CTP, 5 mM GTP, 5 mM TTP, 20 units Human Placental RNAse Inhibitor (New England Biolabs catalog number M0307S), 0.04 units inorganic Pyrophosphatase (Thermo Fisher Scientific catalog number EF0221), 2 nM template DNA and the RNA polymerase amounts specified below. IVT reactions were reacted for 2 hours at 30° C.

Reactions with both RNApols were run either with or without added accessory proteins (SEQ ID NO: 27, 36, 75-77). The approximate amounts of accessory proteins added to each 20 μL IVT reactions were as follows: SEQ ID NO 27:700 ng; SEQ ID NO 75:73 ng; SEQ ID NO 76:280 ng; SEQ ID NO 77:730 ng; SEQ ID NO 36:890 ng.

The following amounts of RNA polymerase were added to the IVT reactions. T7 RNA polymerase with 5 kb template: 100 ng; T7 RNA polymerase with 10 kb template: 200 ng; RNApol137 with 5 kb template: 50 ng; RNApol137 with 10 kb template: 100 ng.

After completion of each reaction, the 20 μL IVT reaction was treated with 2 units DNase I (New England Biolabs catalog number M0303S) in a 30 μL final reaction volume by adding 3 μL 10×DNase I Reaction Buffer (100 mM Tris-HCl pH 7.6 @ 25° C., 25 mM MgCl2, 5 mM CaCl2) 1 μL DNAse I at 2,000 U/μL in storage buffer: (10 mM Tris-HCl, 2 mM CaCl2, 50% Glycerol, pH 7.6 at 25° C.) and 6 μL distilled H₂O. The DNAse I reactions were incubated at 37° C. for 30 min, then placed on ice and stored at −80° C. until placed on an Agilent Corporation (Santa Clara, CA, USA) Fragment Analyzer capillary electrophoresis system used to analyze and quantitate RNA yields.

Based on the Fragment Analyzer quantitation of RNA yields, the results obtained are shown in Tables 2 and 3, with relative total RNA yields represented in Table 2 and relative full-length RNA yields in Table 3. Values from separate reactions were averaged and standard deviations calculated. RNA yields were expressed as % of RNA yields obtained in control reactions without added accessory proteins. All five accessory proteins resulted in higher total RNA yields compared to controls for both RNApols using the 5 kb template. Two of five accessory proteins resulted in higher total RNA yields compared to controls for T7 RNApol (SEQ ID NO:78) using the 10 kb template. Three of five accessory proteins resulted in higher total RNA yields compared to controls for RNApol137 (SEQ ID NO:79) using the 10 kb template. Four of five accessory proteins resulted in higher full-length RNA yields compared to controls for T7 RNApol (SEQ ID NO:78) using the 5 kb template. All five accessory proteins resulted in higher full-length RNA yields compared to controls for RNApol137 (SEQ ID NO:79) using the 5 kb template. Two of five accessory proteins resulted in higher full-length RNA yields compared to controls for T7 RNApol (SEQ ID NO:78) using the 10 kb template. Two of five accessory proteins resulted in higher full-length RNA yields compared to controls for RNApol137 (SEQ ID NO:79) using the 10 kb template.

TABLE 2
Relative total RNA yields from IVT reactions
containing accessory proteins

| | | | | | | Total |
|---|---|---|---|---|---|---|
| | | | | | Total | RNA |
| | | | SEQ | SEQ | RNA | yield, |
| | | | ID NO | ID NO of | yield, | % of |
| Template | | | of RNA | accessory | % of | control |
| SEQ ID | Template | | polymerase | protein in | control | (standard |
| NO | length | | in reaction | reaction | (average) | deviation) |
| 82 | 5 | kb | 78 | 27 | 130.6 | 17.9 |
| 82 | 5 | kb | 78 | 75 | 112.7 | 7.7 |
| 82 | 5 | kb | 78 | 76 | 110.2 | 11.4 |
| 82 | 5 | kb | 78 | 77 | 103.5 | 19.5 |
| 82 | 5 | kb | 78 | 36 | 111.2 | 10.8 |
| 83 | 5 | kb | 79 | 27 | 151.1 | 7.1 |
| 83 | 5 | kb | 79 | 75 | 125.9 | 8.0 |
| 83 | 5 | kb | 79 | 76 | 126.0 | 5.8 |
| 83 | 5 | kb | 79 | 77 | 132.0 | 5.0 |
| 83 | 5 | kb | 79 | 36 | 134.2 | 8.5 |
| 86 | 10 | kb | 78 | 27 | 108.9 | 13.2 |
| 86 | 10 | kb | 78 | 75 | 98.9 | 11.3 |
| 86 | 10 | kb | 78 | 76 | 89.6 | 9.4 |
| 86 | 10 | kb | 78 | 77 | 92.8 | 15.1 |
| 86 | 10 | kb | 78 | 36 | 107.2 | 11.9 |
| 87 | 10 | kb | 79 | 27 | 121.0 | 18.1 |
| 87 | 10 | kb | 79 | 75 | 105.8 | 6.1 |
| 87 | 10 | kb | 79 | 76 | 88.6 | 9.3 |
| 87 | 10 | kb | 79 | 77 | 80.8 | 11.4 |
| 87 | 10 | kb | 79 | 36 | 110.6 | 8.5 |

TABLE 3
Relative full-length RNA yields from IVT
reactions containing accessory proteins

| | | | | | | Full- |
|---|---|---|---|---|---|---|
| | | | | | Full- | length |
| | | | SEQ | SEQ | length | RNA |
| | | | ID NO | ID NO of | RNA | yield, % |
| Template | | | of RNA | accessory | yield, % | of control |
| SEQ ID | Template | | polymerase | protein in | of control | (standard |
| NO | length | | in reaction | reaction | (average) | deviation) |
| 82 | 5 | kb | 78 | 27 | 123.1 | 18.1 |
| 82 | 5 | kb | 78 | 75 | 109.1 | 7.6 |
| 82 | 5 | kb | 78 | 76 | 102.0 | 11.2 |
| 82 | 5 | kb | 78 | 77 | 97.0 | 19.6 |
| 82 | 5 | kb | 78 | 36 | 107.5 | 8.1 |
| 83 | 5 | kb | 79 | 27 | 148.8 | 7.3 |
| 83 | 5 | kb | 79 | 75 | 121.0 | 8.6 |
| 83 | 5 | kb | 79 | 76 | 103.9 | 5.7 |
| 83 | 5 | kb | 79 | 77 | 114.9 | 7.3 |
| 83 | 5 | kb | 79 | 36 | 133.5 | 10.3 |
| 86 | 10 | kb | 78 | 27 | 108.2 | 11.8 |
| 86 | 10 | kb | 78 | 75 | 93.5 | 11.2 |
| 86 | 10 | kb | 78 | 76 | 76.7 | 7.2 |
| 86 | 10 | kb | 78 | 77 | 83.6 | 12.2 |
| 86 | 10 | kb | 78 | 36 | 104.4 | 10.7 |
| 87 | 10 | kb | 79 | 27 | 123.0 | 17.7 |
| 87 | 10 | kb | 79 | 75 | 91.1 | 4.9 |
| 87 | 10 | kb | 79 | 76 | 57.6 | 5.4 |
| 87 | 10 | kb | 79 | 77 | 56.6 | 7.6 |
| 87 | 10 | kb | 79 | 36 | 104.7 | 7.1 |

### Example 2: Increased RNA Yields Obtained from In Vitro Transcription (IVT) Reactions Containing DNA Ligases and Single-Stranded DNA-Binding Proteins (SSBs)

A phylogenetically diverse collection of phage DNA ligases and SSBs were tested for their effects on RNA yield when added to IVT reactions. The genetic diversity of these proteins is illustrated by the distance matrices in Table 4 (DNA ligases) and Table 5 (SSBs). To demonstrate the generality of these effects in IVT reactions, the reactions were performed using four diverse single-subunit RNA polymerases, the genetic diversity of which is illustrated in Table 6. All distance matrices were created using GENEIOUS® version 9.1.6 (available from the world wide web site https://www.geneious.com), based on sequence alignments created using MAFFT version 7.309 (Katoh 2002, Katoh 2013).

RNA polymerases (SEQ ID NO: 78-79), DNA ligases (SEQ ID NO: 27-35), and SSBs (SEQ ID NO: 36-50) were expressed in E. coli and purified for use in the IVT reactions. To produce each purified protein, an expression plasmid containing the gene was transformed into E. coli strain BL21 and a single colony picked for cultivation and protein expression. The bacterial cells were grown in LB medium at 30° C. to log phase culture and induced by addition of L-arabinose. After 18 hours of incubation at 16° C., the cultures were harvested by centrifugation and the collected E. coli cells lysed. Expressed proteins were purified with nickel affinity chromatography using MAGNEHIS™ Ni Particles (Promega catalog number V8565) according to the manufacturer's instructions. Protein was eluted with imidazole solution, concentrated with AMICON® Ultra-centrifugal filter sold by Millipore (Darmstadt, Germany) and transferred to a storage buffer composed of 50 mM Tris pH 8.0, 75 mM NaCl, 0.05 mM EDTA, 10 mM 2-mercaptoethanol, and 50% glycerol. The purified proteins were stored at −20° C. until experimentation.

Two of the RNA polymerases (SEQ ID NO: 80-81) were expressed using a Pseudomonas fluorescens expression system, as previously described (U.S. Pat. Nos. 8,288,127; 8,530,171; and 10,787,671). These two RNA polymerases were purified as described above.

Three of the DNA ligases (SEQ ID NO: 1, 25, 26) were purchased commercially from New England Biolabs (catalog numbers M0317S for SEQ ID NO 25, M0202S for SEQ ID NO 26, and M0318S for SEQ ID NO 1).

IVT reactions were run using either T7 RNA polymerase (SEQ ID NO:78), RNA polymerase 137 (RNApol137, SEQ ID NO:79), RNA polymerase 126 (RNApol126, SEQ ID NO: 80) and RNA polymerase 157 (RNApol157, SEQ ID NO:81), using double-stranded DNA templates incorporating the specific promoters of either polymerase. The DNA templates (SEQ ID NO: 82-85) were PCR amplified and gel-purified using the NucleoSpin Gel and PCR Clean-up kit (Macherey-Nagel catalog number 740609.250).

20 μL IVT reactions were run in triplicate and contained 40 mM Tris-HCl pH 8.0, 6 mM MgCl2, 10 mM dithiothreitol, 2 mM spermidine, 5 mM ATP, 5 mM CTP, 5 mM GTP, 5 mM UTP, 20 units Human Placental RNAse Inhibitor (New England Biolabs catalog number M0307S), 0.04 units inorganic Pyrophosphatase (Thermo Fisher Scientific catalog number EF0221), 2 nM template DNA and the RNA polymerase amounts specified below. IVT reactions were reacted for 2 hours at 30° C.

The following amounts of RNA polymerase were added to the IVT reactions. T7 RNA polymerase: 50 ng; RNApol137:50 ng; RNApol126:100 ng; RNApol157:25 ng.

Reactions with all RNApols were run with either 2 μL (400 ng) accessory protein (SEQ ID NO: 1, 25-50) or 2 μL distilled H2O.

After completion of each reaction, the 20 μL IVT reaction was treated with 2 units DNase I (New England Biolabs catalog number M0303S) in a 30 μL final reaction volume by adding 3 μL 10×DNase I Reaction Buffer (100 mM Tris-HCl pH 7.6 at 25° C., 25 mM MgCl2, 5 mM CaCl₂)), 1 μL DNAse I at 2,000 U/μL in storage buffer (10 mM Tris-HCl, 2 mM CaCl₂), 50% Glycerol, pH 7.6 at 25° C.), and 6 μL distilled H₂O. The DNAse I reactions were incubated at 37° C. for 30 min, then diluted to a 120 μL final volume by adding 90 μL distilled H₂O and placed on ice. Reactions were stored at −80° C. for 1-2 days until the RNA purification step.

10 μL of each 120 μL IVT reaction was purified using the RNA Clean & Concentrator-96 kit (Zymo Research catalog number R1080), eluted in a final volume of 100 μL with distilled H₂O, and stored on ice.

The RNA in each purified IVT reaction was quantified fluorometrically using the QUANTIFLUOR® RNA System (Promega catalog number E3310). QUANTIFLUOR® RNA Dye (Promega catalog number E286A) was diluted 1/400 in 1×TE buffer, pH 7.5 (Promega catalog number E260A), and 200 μL of this dye solution was mixed with either purified IVT reaction or RNA standard. The following amounts of sample or standard were added, depending on the RNA polymerase used in the IVT reaction: T7 RNA polymerase: 10 μL; RNApol137:10 μL; RNApol126:10 μL; RNApol157:15 μL. The RNA standards (500, 250, 125, 62.5, 31.25, 15.625, 7.8125, and 3.90625 ng/μL) were created by performing two-fold serial dilutions on a stock of purified 2-kb, 100 ng/μL mRNA. All dyed samples and standards were prepared in black 96-well assay plates (Corning catalog number CLS3915). The relative fluorescence units from all samples and standards were quantified using a 485/20 excitation and 528/20 emission filter set on the Synergy HTX Multi-mode Reader (BioTek Instruments, Inc catalog number S1LFA). The RNA concentration of each sample was calculated based on a linear equation of standards from the same assay plate.

The results of fluorometric quantitation of RNA yields from the in vitro transcription reactions containing accessory proteins are shown in Table 7. Values from separate replicate reactions were averaged and standard deviations calculated. RNA yields are expressed as the total micrograms RNA produced in each IVT reaction, and the RNA yields for the corresponding H₂O control IVT reactions are provided for comparison. All twelve DNA ligases and fifteen single-stranded DNA-binding proteins resulted in higher RNA yields relative to the H₂O controls, for at least one of the four tested RNApols. IVTs with T7 RNA polymerase showed increased RNA yields with 12 DNA ligases and 15 SSBs. IVTs with RNApol137 or RNApol126 showed increased RNA yields with 12 DNA ligases and 13 SSBs. IVTs with RNApol157 showed increased RNA yields with 12 DNA ligases and 14 SSBs.

TABLE 4
Matrix of percent identities for pairwise amino acid sequence
alignments of DNA ligase proteins. SEQ ID NOs correspond to the
native versions of the sequences, without the added His tag.

| | | DNA ligase SEQ ID NO | | | | | | | | | | |
|---|---|---|---|---|---|---|---|---|---|---|---|---|
| | | 1 | 9 | 8 | 25 | 6 | 3 | 5 | 2 | 7 | 26 | 4 |
| DNA | 1 | | 90.2 | 69.6 | 67.1 | 66.6 | 66.5 | 61.2 | 41.8 | 42.6 | 15.6 | 17.4 |
| Ligase | 9 | 90.2 | | 73.7 | 69.4 | 65.7 | 66.5 | 60.6 | 41.3 | 42.1 | 14.8 | 17.3 |
| SEQ | 8 | 69.6 | 73.7 | | 90.8 | 72.1 | 67.6 | 62.2 | 44.3 | 43.2 | 15.2 | 16.5 |
| ID NO | 25 | 67.1 | 69.4 | 90.8 | | 69.7 | 64.5 | 58.9 | 43.1 | 42.0 | 14.9 | 16.2 |
| | 6 | 66.6 | 65.7 | 72.1 | 69.7 | | 66.3 | 58.3 | 40.8 | 39.8 | 18.3 | 19.1 |
| | 3 | 66.5 | 66.5 | 67.6 | 64.5 | 66.3 | | 61.9 | 41.5 | 40.5 | 16.3 | 16.1 |
| | 5 | 61.2 | 60.6 | 62.2 | 58.9 | 58.3 | 61.9 | | 43.9 | 42.9 | 16.9 | 18.7 |
| | 2 | 41.8 | 41.3 | 44.3 | 43.1 | 40.8 | 41.5 | 43.9 | | 91.1 | 13.8 | 17.4 |
| | 7 | 42.6 | 42.1 | 43.2 | 42.0 | 39.8 | 40.5 | 42.9 | 91.1 | | 13.3 | 16.9 |
| | 26 | 15.6 | 14.8 | 15.2 | 14.9 | 18.3 | 16.3 | 16.9 | 13.8 | 13.3 | | 12.0 |
| | 4 | 17.4 | 17.3 | 16.5 | 16.2 | 19.1 | 16.1 | 18.7 | 17.4 | 16.9 | 12.0 | |

TABLE 5
Matrix of percent identities for pairwise amino acid sequence alignments of SSB proteins.
SEQ ID NOs correspond to the native versions of the sequences, without the added His tag.

| | | Single-stranded DNA binding protein (SSB) SEQ ID NO | | | | | | | | | | | | | | |
|---|---|---|---|---|---|---|---|---|---|---|---|---|---|---|---|---|
| | | 10 | 24 | 23 | 22 | 13 | 21 | 17 | 18 | 20 | 12 | 16 | 19 | 11 | 14 | 15 |
| Single-stranded | 10 | | 98.7 | 98.3 | 92.3 | 92.2 | 82.8 | 65.7 | 65.2 | 60.9 | 60.9 | 59.2 | 44.1 | 26.3 | 17.0 | 17.0 |
| DNA binding | 24 | 98.7 | | 97.8 | 92.3 | 91.8 | 82.8 | 65.2 | 64.8 | 60.5 | 60.1 | 59.2 | 44.1 | 26.3 | 16.6 | 17.0 |
| protein (SSB) | 23 | 98.3 | 97.8 | | 92.3 | 91.8 | 82.4 | 64.8 | 64.4 | 60.5 | 60.1 | 59.2 | 44.1 | 26.3 | 16.2 | 17.0 |
| SEQ ID NO | 22 | 92.3 | 92.3 | 92.3 | | 88.9 | 80.4 | 64.3 | 63.8 | 60.0 | 57.0 | 57.5 | 43.3 | 25.6 | 17.3 | 17.7 |
| | 13 | 92.2 | 91.8 | 91.8 | 88.9 | | 84.1 | 65.1 | 65.1 | 60.3 | 61.0 | 59.3 | 45.1 | 26.0 | 17.2 | 17.6 |
| | 21 | 82.8 | 82.8 | 82.4 | 80.4 | 84.1 | | 65.8 | 65.4 | 59.1 | 59.7 | 57.2 | 45.2 | 25.9 | 16.8 | 16.4 |
| | 17 | 65.7 | 65.2 | 64.8 | 64.3 | 65.1 | 65.8 | | 99.1 | 65.8 | 61.1 | 56.3 | 42.3 | 25.7 | 19.7 | 20.5 |
| | 18 | 65.2 | 64.8 | 64.4 | 63.8 | 65.1 | 65.4 | 99.1 | | 65.8 | 61.5 | 56.3 | 42.3 | 25.7 | 20.1 | 20.9 |
| | 20 | 60.9 | 60.5 | 60.5 | 60.0 | 60.3 | 59.1 | 65.8 | 65.8 | | 61.1 | 58.9 | 41.8 | 26.8 | 20.8 | 18.7 |
| | 12 | 60.9 | 60.1 | 60.1 | 57.0 | 61.0 | 59.7 | 61.1 | 61.5 | 61.1 | | 53.8 | 39.3 | 25.0 | 19.7 | 19.7 |
| | 16 | 59.2 | 59.2 | 59.2 | 57.5 | 59.3 | 57.2 | 56.3 | 56.3 | 58.9 | 53.8 | | 43.1 | 25.4 | 16.9 | 18.2 |
| | 19 | 44.1 | 44.1 | 44.1 | 43.3 | 45.1 | 45.2 | 42.3 | 42.3 | 41.8 | 39.3 | 43.1 | | 26.8 | 16.2 | 16.7 |
| | 11 | 26.3 | 26.3 | 26.3 | 25.6 | 26.0 | 25.9 | 25.7 | 25.7 | 26.8 | 25.0 | 25.4 | 26.8 | | 16.7 | 18.9 |
| | 14 | 17.0 | 16.6 | 16.2 | 17.3 | 17.2 | 16.8 | 19.7 | 20.1 | 20.8 | 19.7 | 16.9 | 16.2 | 16.7 | | 35.0 |
| | 15 | 17.0 | 17.0 | 17.0 | 17.7 | 17.6 | 16.4 | 20.5 | 20.9 | 18.7 | 19.7 | 18.2 | 16.7 | 18.9 | 35.0 | |

TABLE 6
Matrix of percent identities for pairwise amino acid
sequence alignments of the RNA polymerase proteins
used in the IVT reactions. The His tag residues (“HHHHHHGS”) were
removed prior to sequence alignment.

RNApol SEQ ID NO

| | | 78 | 81 | 79 | 80 |
|---|---|---|---|---|---|
| RNApol | 78 | | 61.3 | 29.6 | 30.9 |
| SEQ ID NO | 81 | 61.3 | | 29.1 | 30.1 |
| | 79 | 29.6 | 29.1 | | 45.6 |
| | 80 | 30.9 | 30.1 | 45.6 | |

TABLE 7
Mean RNA yields and standard deviations of in vitro transcription reactions
containing accessory proteins compared to control reactions.

| | | | | | | Control | | |
|---|---|---|---|---|---|---|---|---|
| | | | | Mean | | mean | | Mean % |
| | | | | RNA yield | Control | RNA yield | Mean % | standard |
| RNA | Accessory | Accessory | Mean | standard | mean | standard | RNA yield, | deviation, |
| polymerase | protein | protein | RNA yield | deviation | RNA yield | deviation | relative | relative |
| SEQ ID NO | SEQ ID NO | type | (μg) | (μg) | (μg) | (μg) | to control | to control |
| 78 | 1 | Ligase | 8.42 | 1.12 | 6.46 | 0.52 | 130.43 | 17.30 |
| 78 | 25 | Ligase | 8.61 | 0.66 | 6.46 | 0.52 | 133.40 | 10.20 |
| 78 | 26 | Ligase | 9.09 | 0.92 | 6.46 | 0.52 | 140.83 | 14.32 |
| 78 | 27 | Ligase | 8.56 | 0.79 | 6.46 | 0.52 | 132.66 | 12.26 |
| 78 | 28 | Ligase | 8.47 | 0.38 | 6.46 | 0.52 | 131.18 | 5.89 |
| 78 | 29 | Ligase | 7.89 | 0.75 | 6.46 | 0.52 | 122.27 | 11.57 |
| 78 | 30 | Ligase | 8.76 | 1.01 | 6.46 | 0.52 | 135.63 | 15.59 |
| 78 | 31 | Ligase | 7.99 | 1.30 | 6.46 | 0.52 | 123.75 | 20.21 |
| 78 | 32 | Ligase | 8.42 | 0.79 | 6.46 | 0.52 | 130.43 | 12.26 |
| 78 | 33 | Ligase | 8.33 | 0.52 | 6.46 | 0.52 | 128.95 | 8.03 |
| 78 | 34 | Ligase | 8.90 | 0.76 | 6.46 | 0.52 | 137.86 | 11.78 |
| 78 | 35 | Ligase | 8.23 | 1.16 | 6.46 | 0.52 | 127.47 | 18.00 |
| 78 | 36 | SSB | 7.94 | 1.09 | 6.46 | 0.52 | 123.01 | 16.86 |
| 78 | 37 | SSB | 8.13 | 0.54 | 6.46 | 0.52 | 125.98 | 8.43 |
| 78 | 38 | SSB | 8.09 | 0.88 | 6.46 | 0.52 | 125.24 | 13.61 |
| 78 | 39 | SSB | 8.56 | 1.16 | 6.46 | 0.52 | 132.66 | 18.00 |
| 78 | 40 | SSB | 7.61 | 0.29 | 6.46 | 0.52 | 117.82 | 4.45 |
| 78 | 41 | SSB | 8.33 | 1.25 | 6.46 | 0.52 | 128.95 | 19.41 |
| 78 | 42 | SSB | 8.13 | 1.08 | 6.46 | 0.52 | 125.98 | 16.71 |
| 78 | 43 | SSB | 7.85 | 0.79 | 6.46 | 0.52 | 121.53 | 12.26 |
| 78 | 44 | SSB | 7.75 | 0.66 | 6.46 | 0.52 | 120.04 | 10.20 |
| 78 | 45 | SSB | 8.76 | 0.29 | 6.46 | 0.52 | 135.63 | 4.45 |
| 78 | 46 | SSB | 7.75 | 0.66 | 6.46 | 0.52 | 120.04 | 10.20 |
| 78 | 47 | SSB | 8.23 | 0.79 | 6.46 | 0.52 | 127.47 | 12.26 |
| 78 | 48 | SSB | 7.80 | 0.36 | 6.46 | 0.52 | 120.78 | 5.60 |
| 78 | 49 | SSB | 7.70 | 0.60 | 6.46 | 0.52 | 119.30 | 9.27 |
| 78 | 50 | SSB | 8.76 | 0.25 | 6.46 | 0.52 | 135.63 | 3.86 |
| 79 | 1 | Ligase | 6.39 | 0.38 | 5.97 | 0.51 | 107.14 | 6.29 |
| 79 | 25 | Ligase | 6.63 | 0.08 | 5.97 | 0.51 | 111.10 | 1.37 |
| 79 | 26 | Ligase | 6.53 | 0.51 | 5.97 | 0.51 | 109.51 | 8.58 |
| 79 | 27 | Ligase | 6.44 | 0.57 | 5.97 | 0.51 | 107.93 | 9.61 |
| 79 | 28 | Ligase | 6.30 | 0.33 | 5.97 | 0.51 | 105.55 | 5.49 |
| 79 | 29 | Ligase | 6.20 | 0.50 | 5.97 | 0.51 | 103.96 | 8.35 |
| 79 | 30 | Ligase | 6.49 | 0.16 | 5.97 | 0.51 | 108.72 | 2.75 |
| 79 | 31 | Ligase | 6.63 | 0.43 | 5.97 | 0.51 | 111.10 | 7.27 |
| 79 | 32 | Ligase | 6.58 | 0.16 | 5.97 | 0.51 | 110.31 | 2.75 |
| 79 | 33 | Ligase | 6.77 | 0.36 | 5.97 | 0.51 | 113.48 | 5.99 |
| 79 | 34 | Ligase | 6.68 | 0.51 | 5.97 | 0.51 | 111.89 | 8.58 |
| 79 | 35 | Ligase | 6.72 | 0.30 | 5.97 | 0.51 | 112.68 | 4.95 |
| 79 | 36 | SSB | 6.06 | 0.50 | 5.97 | 0.51 | 101.59 | 8.35 |
| 79 | 37 | SSB | 6.30 | 0.30 | 5.97 | 0.51 | 105.55 | 4.95 |
| 79 | 38 | SSB | 5.97 | 0.14 | 5.97 | 0.51 | 100.00 | 2.38 |
| 79 | 39 | SSB | 6.06 | 0.16 | 5.97 | 0.51 | 101.59 | 2.75 |
| 79 | 40 | SSB | 4.64 | 0.08 | 5.97 | 0.51 | 77.80 | 1.37 |
| 79 | 41 | SSB | 6.49 | 0.59 | 5.97 | 0.51 | 108.72 | 9.90 |
| 79 | 42 | SSB | 6.35 | 0.46 | 5.97 | 0.51 | 106.34 | 7.65 |
| 79 | 43 | SSB | 6.20 | 0.50 | 5.97 | 0.51 | 103.96 | 8.35 |
| 79 | 44 | SSB | 6.01 | 0.36 | 5.97 | 0.51 | 100.79 | 5.99 |
| 79 | 45 | SSB | 6.44 | 0.36 | 5.97 | 0.51 | 107.93 | 5.99 |
| 79 | 46 | SSB | 6.06 | 0.08 | 5.97 | 0.51 | 101.59 | 1.37 |
| 79 | 47 | SSB | 6.25 | 0.25 | 5.97 | 0.51 | 104.76 | 4.12 |
| 79 | 48 | SSB | 6.25 | 0.25 | 5.97 | 0.51 | 104.76 | 4.12 |
| 79 | 49 | SSB | 6.30 | 0.16 | 5.97 | 0.51 | 105.55 | 2.75 |
| 79 | 50 | SSB | 6.44 | 0.08 | 5.97 | 0.51 | 107.93 | 1.37 |
| 80 | 1 | Ligase | 3.13 | 0.32 | 2.53 | 0.27 | 123.80 | 12.83 |
| 80 | 25 | Ligase | 3.00 | 0.20 | 2.53 | 0.27 | 118.70 | 7.79 |
| 80 | 26 | Ligase | 3.00 | 0.15 | 2.53 | 0.27 | 118.70 | 5.89 |
| 80 | 27 | Ligase | 2.87 | 0.32 | 2.53 | 0.27 | 113.60 | 12.83 |
| 80 | 28 | Ligase | 3.00 | 0.27 | 2.53 | 0.27 | 118.70 | 10.62 |
| 80 | 29 | Ligase | 2.83 | 0.34 | 2.53 | 0.27 | 111.90 | 13.49 |
| 80 | 30 | Ligase | 3.08 | 0.00 | 2.53 | 0.27 | 122.10 | 0.00 |
| 80 | 31 | Ligase | 2.74 | 0.27 | 2.53 | 0.27 | 108.50 | 10.62 |
| 80 | 32 | Ligase | 3.17 | 0.07 | 2.53 | 0.27 | 125.50 | 2.94 |
| 80 | 33 | Ligase | 2.78 | 0.20 | 2.53 | 0.27 | 110.20 | 7.79 |
| 80 | 34 | Ligase | 2.91 | 0.32 | 2.53 | 0.27 | 115.30 | 12.83 |
| 80 | 35 | Ligase | 3.04 | 0.30 | 2.53 | 0.27 | 120.40 | 11.78 |
| 80 | 36 | SSB | 2.87 | 0.30 | 2.53 | 0.27 | 113.60 | 11.78 |
| 80 | 37 | SSB | 2.74 | 0.15 | 2.53 | 0.27 | 108.50 | 5.89 |
| 80 | 38 | SSB | 2.74 | 0.32 | 2.53 | 0.27 | 108.50 | 12.83 |
| 80 | 39 | SSB | 2.66 | 0.27 | 2.53 | 0.27 | 105.10 | 10.62 |
| 80 | 40 | SSB | 2.01 | 0.30 | 2.53 | 0.27 | 79.60 | 11.78 |
| 80 | 41 | SSB | 3.00 | 0.37 | 2.53 | 0.27 | 118.70 | 14.72 |
| 80 | 42 | SSB | 2.87 | 0.15 | 2.53 | 0.27 | 113.60 | 5.89 |
| 80 | 43 | SSB | 2.53 | 0.49 | 2.53 | 0.27 | 100.00 | 19.31 |
| 80 | 44 | SSB | 2.70 | 0.13 | 2.53 | 0.27 | 106.80 | 5.10 |
| 80 | 45 | SSB | 3.00 | 0.27 | 2.53 | 0.27 | 118.70 | 10.62 |
| 80 | 46 | SSB | 2.57 | 0.26 | 2.53 | 0.27 | 101.70 | 10.20 |
| 80 | 47 | SSB | 2.70 | 0.13 | 2.53 | 0.27 | 106.80 | 5.10 |
| 80 | 48 | SSB | 2.57 | 0.13 | 2.53 | 0.27 | 101.70 | 5.10 |
| 80 | 49 | SSB | 2.74 | 0.27 | 2.53 | 0.27 | 108.50 | 10.62 |
| 80 | 50 | SSB | 2.87 | 0.30 | 2.53 | 0.27 | 113.60 | 11.78 |
| 81 | 1 | Ligase | 3.44 | 0.14 | 2.74 | 0.05 | 125.41 | 5.06 |
| 81 | 25 | Ligase | 3.74 | 0.14 | 2.74 | 0.05 | 136.46 | 5.06 |
| 81 | 26 | Ligase | 3.65 | 0.29 | 2.74 | 0.05 | 133.15 | 10.65 |
| 81 | 27 | Ligase | 2.98 | 0.55 | 2.74 | 0.05 | 108.84 | 20.25 |
| 81 | 28 | Ligase | 3.10 | 0.41 | 2.74 | 0.05 | 113.26 | 14.95 |
| 81 | 29 | Ligase | 3.28 | 0.14 | 2.74 | 0.05 | 119.89 | 5.06 |
| 81 | 30 | Ligase | 2.80 | 0.37 | 2.74 | 0.05 | 102.21 | 13.40 |
| 81 | 31 | Ligase | 3.04 | 0.18 | 2.74 | 0.05 | 111.05 | 6.63 |
| 81 | 32 | Ligase | 3.10 | 0.32 | 2.74 | 0.05 | 113.26 | 11.64 |
| 81 | 33 | Ligase | 3.04 | 0.33 | 2.74 | 0.05 | 111.05 | 11.95 |
| 81 | 34 | Ligase | 3.38 | 0.10 | 2.74 | 0.05 | 123.20 | 3.83 |
| 81 | 35 | Ligase | 3.38 | 0.28 | 2.74 | 0.05 | 123.20 | 10.13 |
| 81 | 36 | SSB | 3.10 | 0.14 | 2.74 | 0.05 | 113.26 | 5.06 |
| 81 | 37 | SSB | 2.83 | 0.38 | 2.74 | 0.05 | 103.31 | 13.80 |
| 81 | 38 | SSB | 2.83 | 0.19 | 2.74 | 0.05 | 103.31 | 6.90 |
| 81 | 39 | SSB | 2.92 | 0.29 | 2.74 | 0.05 | 106.63 | 10.65 |
| 81 | 40 | SSB | 1.74 | 0.05 | 2.74 | 0.05 | 63.54 | 1.91 |
| 81 | 41 | SSB | 3.10 | 0.32 | 2.74 | 0.05 | 113.26 | 11.64 |
| 81 | 42 | SSB | 2.95 | 0.16 | 2.74 | 0.05 | 107.73 | 5.74 |
| 81 | 43 | SSB | 2.98 | 0.34 | 2.74 | 0.05 | 108.84 | 12.55 |
| 81 | 44 | SSB | 2.89 | 0.28 | 2.74 | 0.05 | 105.52 | 10.13 |
| 81 | 45 | SSB | 2.83 | 0.34 | 2.74 | 0.05 | 103.31 | 12.55 |
| 81 | 46 | SSB | 2.98 | 0.42 | 2.74 | 0.05 | 108.84 | 15.31 |
| 81 | 47 | SSB | 2.95 | 0.45 | 2.74 | 0.05 | 107.73 | 16.57 |
| 81 | 48 | SSB | 3.04 | 0.40 | 2.74 | 0.05 | 111.05 | 14.45 |
| 81 | 49 | SSB | 3.04 | 0.16 | 2.74 | 0.05 | 111.05 | 5.74 |
| 81 | 50 | SSB | 2.89 | 0.14 | 2.74 | 0.05 | 105.52 | 5.06 |

Ligase: DNA ligase; SSB: single-stranded DNA binding protein.

### REFERENCES

Amiott E A, Jaehning J A (2007). In vitro analysis of the yeast mitochondrial RNA polymerase. Methods Mol Biol. 372:193-206.
Aramburu J, Navas-Castillo J, Moreno P, Cambra M (1991). Detection of double-stranded RNA by ELISA and dot immunobinding assay using an antiserum to synthetic polynucleotides. J Virol Methods 33(1-2):1-11.
Arnaud-Barbe N, Cheynet-Sauvion V, Oriol G, Mandrand B, Mallet F (1998). Transcription of RNA templates by T7 RNA polymerase. Nucleic Acids Res. 26(15):3550-3554.
Arnold J J, Smidansky E D, Moustafa I M, Cameron C E (2012). Human mitochondrial RNA polymerase: structure-function, mechanism and inhibition. Biochim Biophys Acta 1819(9-10):948-960.
Baiersdörfer M, Boros G, Muramatsu H, Mahiny A, Vlatkovic I, Sahin U, Karikó K (2019). A Facile Method for the Removal of dsRNA Contaminant from In Vitro-Transcribed mRNA. Mol Ther Nucleic Acids 15:26-35.
Barbier A J, Jiang A Y, Zhang P, Wooster R, Anderson D G (2022). The clinical progress of mRNA vaccines and immunotherapies. Nat Biotechnol. 40(6):840-854.
Bestwick M L, Shadel G S (2013). Accessorizing the human mitochondrial transcription machinery. Trends Biochem Sci. 38(6):283-291.
Borkotoky S, Murali A (2018). The highly efficient T7 RNA polymerase: A wonder macromolecule in biological realm. Int J Biol Macromol. 118 (Pt A): 49-56.
Börner T, Aleynikova A Y, Zubo Y O, Kusnetsov V V (2015). Chloroplast RNA polymerases: Role in chloroplast biogenesis. Biochim Biophys Acta. 1847(9):761-769.
Cazenave C, Uhlenbeck O C (1994). RNA template-directed RNA synthesis by T7 RNA polymerase. Proc Natl Acad Sci USA 91(15):6972-6976.
Chaudhary N, Weissman D, Whitehead K A (2021). mRNA vaccines for infectious diseases: principles, delivery and clinical translation. Nat Rev Drug Discov. 20(11):817-838.
Darzynkiewicz E, Stepinski J, Ekiel I, Jin Y, Haber D, Sijuwade T, Tahara S M (1988). Beta-globin mRNAs capped with m7G, m2.7 (2) G or m2.2.7 (3) G differ in intrinsic translation efficiency. Nucleic Acids Res. 16(18):8953-62.
Deshpande A P, Patel S S (2012). Mechanism of transcription initiation by the yeast mitochondrial RNA polymerase. Biochim Biophys Acta 1819(9-10):930-938.
Di Grandi D, Dayeh D M, Kaur K, Chen Y, Henderson S, Moon Y, Bhowmick A, Ihnat P M, Fu Y, Muthusamy K, Palackal N, Pyles E A (2023). A single-nucleotide resolution capillary gel electrophoresis workflow for poly (A) tail characterization in the development of mRNA therapeutics and vaccines. J Pharm Biomed Anal. 236:115692.
Diken M, Kranz L M, Kreiter S, Sahin U (2017). mRNA: A Versatile Molecule for Cancer Vaccines. Curr Issues Mol Biol. 22:113-128.
Dolgin E (2019). Unlocking the potential of vaccines built on messenger RNA. Nature 574(7778):S10-S12.
Durniak K J, Bailey S, Steitz T A (2008). The structure of a transcribing T7 RNA polymerase in transition from initiation to elongation. Science 322(5901):553-557.
Fiedler K, Lazzaro S, Lutz J, Rauch S, Heidenreich R (2016). mRNA Cancer Vaccines. Recent Results Cancer Res. 209:61-85.
Fisher R P, Lisowsky T, Parisi M A, Clayton D A (1992). DNA wrapping and bending by a mitochondrial high mobility group-like transcriptional activator protein. J Biol Chem. 267(5):3358-3367.
Gandhi V, O'Brien M H, Yadav S (2020). High-quality and high-yield RNA extraction method from whole human saliva. Biomark Insights. 15:1177271920929705.
Gantier M P, Williams B R (2007). The response of mammalian cells to double-stranded RNA. Cytokine Growth Factor Rev. 18(5-6):363-71.
Ge Q, Dallas A, Ilves H, Shorenstein J, Behlke M A, Johnston B H (2010). Effects of chemical modification on the potency, serum stability, and immunostimulatory properties of short shRNAs. RNA 16(1):118-130.
Gholamalipour Y, Karunanayake Mudiyanselage A, Martin C T (2018). 3′ end additions by T7 RNA polymerase are RNA self-templated, distributive and diverse in character-RNA-Seq analyses. Nucleic Acids Res. 46(18):9253-9263.
Grudzien-Nogalska E, Kowalska J, Su W, Kuhn A N, Slepenkov S V, Darzynkiewicz E, Sahin U, Jemielity J, Rhoads R E (2013). Synthetic mRNAs with superior translation and stability properties. Methods Mol Biol. 969:55-72.
Grunwitz C, Kranz L M (2017). mRNA Cancer Vaccines-Messages that Prevail. Curr Top Microbiol Immunol. 405:145-164.
Gualberto J M, Kühn K (2014). DNA-binding proteins in plant mitochondria: implications for transcription. Mitochondrion 19 Pt B: 323-328.
Gunter H M, Idrisoglu S, Singh S, Han D J, Ariens E, Peters J R, Wong T, Cheetham S W, Xu J, Rai S K, Feldman R, Herbert A, Marcellin E, Tropee R, Munro T, Mercer T R (2023). mRNA vaccine quality analysis using RNA sequencing. Nat Commun. 14(1):5663.
Hadi M, Stacy E A (2023). An optimized RNA extraction method for diverse leaves of Hawaiian Metrosideros, a hypervariable tree species complex. Appl Plant Sci. 11(3):e11518.
Henderson J M, Ujita A, Hill E, Yousif-Rosales S, Smith C, Ko N, McReynolds T, Cabral C R, Escamilla-Powers J R, Houston M E (2021). Cap 1 messenger RNA synthesis with co-transcriptional CleanCap® analog by In Vitro Transcription. Curr Protoc. 1(2):e39.
Hogan M J, Pardi N (2022). mRNA Vaccines in the COVID-19 Pandemic and Beyond. Annu. Rev Med. 73:17-39.
Huang Y, Eckstein F, Padilla R, Sousa R (1997). Mechanism of ribose 2′-group discrimination by an RNA polymerase. Biochemistry 36(27):8231-8242.
Iavarone C, O'hagan D T, Yu D, Delahaye N F, Ulmer J B (2017). Mechanism of action of mRNA-based vaccines. Expert Rev Vaccines. 16(9):871-881.
Jackson A L, Burchard J, Leake D, Reynolds A, Schelter J, Guo J, Johnson J M, Lim L, Karpilow J, Nichols K, Marshall W, Khvorova A, Linsley P S (2006). Position-specific chemical modification of siRNAs reduces “off-target” transcript silencing. RNA 12(7):1197-1205.
Jang S H, Jaehning J A (1991). The yeast mitochondrial RNA polymerase specificity factor, MTF1, is similar to bacterial sigma factors. J Biol Chem. 266(33):22671-22677.
Karikó K, Ni H, Capodici J, Lamphier M, Weissman D (2004). mRNA is an endogenous ligand for Toll-like receptor 3. J. Biol. Chem. 279(13), 12542-12550.
Karikó K, Muramatsu H, Welsh F A, Ludwig J, Kato H, Akira S, Weissman D (2008). Incorporation of pseudouridine into mRNA yields superior nonimmunogenic vector with increased translational capacity and biological stability. Mol Ther. 16(11):1833-1840.
Karikó K, Muramatsu H, Ludwig J, Weissman D (2011). Generating the optimal mRNA for therapy: HPLC purification eliminates immune activation and improves translation of nucleoside-modified, protein-encoding mRNA. Nucl. Acids Res. 39(21), e142.
Karimkhanilouyi S, Ghorbian S (2019). Nucleic acid vaccines for hepatitis B and C virus. Infect Genet Evol. 75:103968.
Katoh K, Misawa K, Kuma K, Miyata T (2002). MAFFT: a novel method for rapid multiple sequence alignment based on fast Fourier transform. Nucl. Acids Res. 30(14): 3059-3066.
Katoh K, Standley D M (2013). MAFFT Multiple Sequence Alignment Software Version 7: Improvements in Performance and Usability, Mol. Biol. Evol. 30(4):772-780.
Kole R, Krainer A R, Altman S (2012). RNA therapeutics: beyond RNA interference and antisense oligonucleotides. Nat Rev Drug Discov. 11(2):125-140.
Konarska M M, Sharp P A (1989). Replication of RNA by the DNA-dependent RNA polymerase of phage T7. Cell 57(3):423-431.
Kramps T, Elbers K (2017). Introduction to RNA Vaccines. Methods Mol Biol. 1499:1-11.
Kraynack B A, Baker B F (2005). Small interfering RNAs containing full 2′-O-methylribonucleotide-modified sense strands display Argonaute2/eIF2C2-dependent activity. RNA 12(1):163-176.
Kuhn A N, Diken M, Kreiter S, Vallazza B, Türeci Ö, Sahin U (2011). Determinants of intracellular RNA pharmacokinetics: Implications for RNA-based immunotherapeutics. RNA Biol. 8(1):35-43.
Kuhn A N, Beißert T, Simon P, Vallazza B, Buck J, Davies B P, Tureci O, Sahin U (2012). mRNA as a versatile tool for exogenous protein expression. Curr Gene Ther. 12(5):347-361.
Kühn K, Bohne A V, Liere K, Weihe A, Börner T. (2007). Arabidopsis phage-type RNA polymerases: accurate in vitro transcription of organellar genes. Plant Cell 19(3):959-971.
Layzer J M, McCaffrey A P, Tanner A K, Huang Z, Kay M A, Sullenger B A (2004). In vivo activity of nuclease-resistant siRNAs. RNA 10(5):766-771.
Lengyel P (1987). Double-stranded RNA and interferon action. J Interferon Res. 7(5):511-519.
Majde J A (2000). Viral double-stranded RNA, cytokines, and the flu. J Interferon Cytokine Res. 20(3):259-272.
Majlessi M, Nelson N C, Becker M M (1998). Advantages of 2′-O-methyl oligoribonucleotide probes for detecting RNA targets. Nucleic Acids Res. 26(9):2224-2229.
Martin C T, Muller D K, Coleman J E (1988). Processivity in early stages of transcription by T7 RNA polymerase. Biochemistry 27(11):3966-3974.
Matsunaga M, Jaehning J A (2004). Intrinsic promoter recognition by a “core” RNA polymerase. J Biol Chem. 279(43):44239-44242.
Mu X, Greenwald E, Ahmad S, Hur S (2018). An origin of the immunogenicity of in vitro transcribed RNA. Nucleic Acids Res. 46(10):5239-5249.
Nayak D, Guo Q, Sousa R (2007). Functional architecture of T7 RNA polymerase transcription complexes. J Mol Biol. 371(2):490-500.
Paratkar S, Deshpande A P, Tang G Q, Patel S S (2011). The N-terminal domain of the yeast mitochondrial RNA polymerase regulates multiple steps of transcription. J Biol Chem. 286(18):16109-16120.
Pardi N, Hogan M J, Porter F W, Weissman D (2018). mRNA vaccines—a new era in vaccinology. Nat Rev Drug Discov. 17(4):261-279.
Pasquinelli A E, Dahlberg J E, Lund E (1995). Reverse 5′ caps in RNAs made in vitro by phage RNA polymerases. RNA 1(9):957-967.
Pfannschmidt T, Blanvillain R, Merendino L, Courtois F, Chevalier F, Liebers M, Grübler B, Hommel E, Lerbs-Mache S (2015). Plastid RNA polymerases: orchestration of enzymes with different evolutionary origins controls chloroplast biogenesis during the plant life cycle. J. Exp. Botany 66(22):6957-6973.
Posse V, Gustafsson C M (2017). Human Mitochondrial Transcription Factor B2 Is Required for Promoter Melting during Initiation of Transcription. J Biol Chem. 292(7):2637-2645.
Poveda C, Biter A B, Bottazzi M E, Strych U (2019). Establishing preferred product characterization for the evaluation of RNA vaccine antigens. Vaccines 7(4), 131.
Qin S, Tang X, Chen Y, Chen K, Fan N, Xiao W, Zheng Q, Li G, Teng Y, Wu M, Song X (2022). mRNA-based therapeutics: powerful and versatile tools to combat diseases. Signal Transduct Target Ther. 7(1):166.
Rohner E, Yang R, Foo K S, Goedel A, Chien K R (2022). Unlocking the promise of mRNA therapeutics. Nat Biotechnol. 40(11):1586-1600.
Rosa S S, Prazeres D M F, Azevedo A M, Marques M P C (2021). mRNA vaccines manufacturing: Challenges and bottlenecks. Vaccine 39(16):2190-2200.
Rueden C T, Schindelin J, Hiner M C, DeZonia B E, Walter A E, Arena E T, Eliceiri K W (2017). ImageJ2: ImageJ for the next generation of scientific image data. BMC Bioinformatics, 18(1). doi: 10.1186/s12859-017-1934-z
Sahin U, Karikó K, Türeci Ö (2014). mRNA-based therapeutics—developing a new class of drugs. Nat Rev Drug Discov. 13(10):759-780.
Scheiblhofer S, Thalhamer J, Weiss R (2018). DNA and mRNA vaccination against allergies. Pediatr. Allergy Immunol. 29(7):679-688.
Schindelin J, Arganda-Carreras I, Frise E, Kaynig V, Longair M, Pietzsch T, Cardona A (2012). Fiji: an open-source platform for biological-image analysis. Nature Methods, 9(7), 676-682.
Schneider C A, Rasband W S, Eliceiri, K W (2012). NIH Image to ImageJ: 25 years of image analysis. Nature Methods, 9(7), 671-675.
Schönborn J, Oberstrass J, Breyel E, Tittgen J, Schumacher J, Lukacs N (1991). Monoclonal antibodies to double-stranded RNA as probes of RNA structure in crude nucleic acid extracts. Nucleic Acids Res. 19(11):2993-3000.
Sergeeva O V, Koteliansky V E, Zatsepin T S (2016). mRNA-Based Therapeutics Advances and Perspectives. Biochemistry (Mosc) 81(7):709-22.
Shanmugasundaram M, Senthilvelan A, Anilkumar R K (2022). Recent Advances in Modified Cap Analogs: Synthesis, Biochemical Properties, and mRNA Based Vaccines. Chem Rec. 22(8): e202200005.
Son, K N, Liang, Z G, and Lipton, H L (2015). Double-stranded RNA is detected by immunofluorescence analysis in RNA and DNA virus infections, including those by negative-stranded RNA viruses. J. Virol. 89(18), 9383-9392.
Sousa R, Mukherjee S (2003). T7 RNA polymerase. Prog Nucleic Acid Res Mol Biol. 2003; 73:1-41.
Stark G R, Kerr I M, Williams B R, Silverman R H, Schreiber R D (1998). How cells respond to interferons. Annu Rev Biochem. 67:227-264.
Stepinski J, Waddell C, Stolarski R, Darzynkiewicz E, Rhoads R E (2001). Synthesis and properties of mRNAs containing the novel “anti-reverse” cap analogs 7-methyl (3′-O-methyl) GpppG and 7-methyl (3′-deoxy) GpppG. RNA 7(10):1486-1495.
Sullenger B A, Nair S (2016). From the RNA world to the clinic. Science 352(6292):1417-1420.
Tu Y, Das A, Redwood-Sawyerr C, Polizzi K M (2024). Capped or uncapped? Techniques to assess the quality of mRNA molecules. Curr. Opin. Systems Biol. 37:100503.
Triana-Alonso F J, Dabrowski M, Wadzack J, Nierhaus K H (1995). Self-coded 3′-extension of run-off transcripts produces aberrant products during in vitro transcription with T7 RNA polymerase. J Biol Chem. 270(11):6298-6307.
Ulmer J B, Geall A J (2016). Recent innovations in mRNA vaccines. Curr Opin Immunol. 41:18-22.
Velazquez G, Guo Q, Wang L, Brieba L G, Sousa R (2012). Conservation of promoter melting mechanisms in divergent regions of the single-subunit RNA polymerases. Biochemistry 51(18):3901-3910.
Xu B, Clayton D A (1992). Assignment of a yeast protein necessary for mitochondrial transcription initiation. Nucleic Acids Res. 20(5):1053-1059.
Warminski M, Mamot A, Depaix A, Kowalska J, Jemielity J (2023). Chemical modifications of mRNA ends for therapeutic applications. Acc. Chem. Res. 56(20):2814-2826.
Warzak D A, Pike W A, Luttgeharm K D (2023). Capillary electrophoresis methods for determining the IVT mRNA critical quality attributes of size and purity. SLAS Technol. 28(5):369-374.
Whitley J, Zwolinski C, Denis C, Maughan M, Hayles L, Clarke D, Snare M, Liao H, Chiou S, Marmura T, Zoeller H, Hudson B, Peart J, Johnson M, Karlsson A, Wang Y, Nagle C, Harris C, Tonkin D, Fraser S, Capiz L, Zeno C L, Meli Y, Martik D, Ozaki D A, Caparoni A, Dickens J E, Weissman D, Saunders K O, Haynes B F, Sempowski G D, Denny T N, Johnson M R (2022). Development of mRNA manufacturing for vaccines and therapeutics: mRNA platform requirements and development of a scalable production process to support early phase clinical trials. Transl Res. 242:38-55.
Wilson C, Keefe A D (2006). Building oligonucleotide therapeutics using non-natural chemistries. Curr Opin Chem Biol. 10(6):607-614.
Zhang C, Maruggi G, Shan H, Li J (2019). Advances in mRNA Vaccines for Infectious Diseases. Front Immunol. 10:594.
Ziemniak M, Strenkowska M, Kowalska J, Jemielity J (2013). Potential therapeutic applications of RNA cap analogs. Future Med Chem. 5(10):1141-1172.

We claim:

1. A method of performing in vitro transcription comprising the steps of
(a) combining a single-subunit RNA polymerase (RNApol) molecule with a double-stranded DNA template and at least one accessory protein and
(b) producing a transcript or a population of transcripts.

2. The method according to claim 1 wherein the at least one accessory protein is a DNA ligase.

3. The method according to claim 1 wherein the at least one accessory protein is a single-stranded DNA binding protein.

4. The method according to claim 1 wherein the at least one accessory protein is a nucleoside monophosphate kinase.

5. The method according to claim 2 wherein the at least one accessory protein has 85% or higher sequence identity to a member of the group consisting of accessory proteins given in SEQ ID NO: 34, SEQ ID NOs: 1-9, SEQ ID NOs: 25-33, SEQ ID NO:35 or SEQ ID NOs: 51-59.

6. The method according to claim 3 wherein the at least one accessory protein has 85% or higher sequence identity to a member of the group consisting of accessory proteins given in SEQ ID NO: 41, SEQ ID NOs: 10-24, SEQ ID NOs: 36-40 or SEQ ID NOs: 42-50, SEQ ID NOs: 60-74.

7. The method according to claim 1 wherein the at least one accessory protein has 75% or higher sequence identity to a member of the group consisting of accessory proteins given in SEQ ID NO: 34, SEQ ID NO:41, SEQ ID NO: 27, SEQ ID NO: 36, SEQ ID NO: 75, SEQ ID NO: 76 and SEQ ID NO: 77.

8. The method according to claim 1 wherein the at least one accessory protein is selected from the group consisting of accessory proteins given in SEQ ID NO:34, SEQ ID NO:41, SEQ ID NO: 27, SEQ ID NO: 36, SEQ ID NO: 75, SEQ ID NO: 76 and SEQ ID NO: 77.

9. The method according to claim 2 wherein the at least one accessory protein is a DNA ligase encoded by a bacteriophage.

10. The method according to claim 2 wherein the at least one accessory protein is a DNA ligase encoded by a prokaryote.

11. The method according to claim 3 wherein the at least one accessory protein is a single-stranded DNA binding protein encoded by a bacteriophage.

12. The method according to claim 3 wherein the at least one accessory protein is a single-stranded DNA binding protein encoded by a prokaryote.

13. The method according to claim 4 wherein the at least one accessory protein is a nucleoside monophosphate kinase encoded by a bacteriophage.

14. The method according to claim 4 wherein the at least one accessory protein is a nucleoside monophosphate kinase encoded by a prokaryote.

15. The method according to claim 1 wherein activity of the RNA polymerase is improved compared to the activity of the RNA polymerase in the absence of the accessory protein, wherein the improved activity of the RNA polymerase is one or more of the group consisting of: increased RNA yield, increased RNA integrity, reduced double stranded RNA, reduced short or truncated RNAs, increased capping efficiency, increased cap incorporation efficiency and increased fidelity.

16. The method according to claim 15 wherein the activity of the RNA polymerase is improved 5% or more.

17. The method according to claim 15 wherein the activity of the RNA polymerase is improved 10% or more.
